SEQUENCE LISTING (10 sequences)

SEQ ID NO: 1 - DNA, 22 bp, Artificial Sequence
  Primer Sequence
tgcccagtacatgaccttatgg

SEQ ID NO: 2 - DNA, 21 bp, Artificial Sequence
  Primer Sequence
gaaatccccgtgagtcaaacc

SEQ ID NO: 3 - DNA, 1168 bp, Artificial Sequence
  Plasmid pMH302
cccgcgaaattaatacgactcactatagggagaccacaacggtttccctctagaaataattttgtttaactttaagaaggagatatacatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagagcggtgctggctccgcaggttctgctggtatgatgttcttaccaaccgattattgttgcaggttaagcgatcaagagtatatggagcttgtgtttgagaatggccagattcttgcaaagggccaaagatccaacgtttctctgcataatcaacgtaccaaatcgatcatggatttgtatgaggcagagtataacgaggatttcatgaagagtatcatccatggtggtggtggtgccatcacaaatctcggggacacgcaggttgttccacaaagtcatgttgctgctgcccatgaaacaaacatgttggaaagcaataaacatgttgaccatcatcaccatcaccattaaaagcttgatc

SEQ ID NO: 4 - DNA, 4277 bp, Artificial Sequence
  Plasmid pHJW156
attaggaaattaatacgactcactataggggaattgtgagcggataacaattcccctgtagaaataattttgtttaactttaataaggagatataccatggttagcggtgttggtggtagcggtggtggtcgtggtggcggtcgcggaggtgaagaagaaccgagcagcagccataccccgaataatcgccgtggtggtgaacaggcacagagcagcggcaccaaaagcctgcgtccgcgtagcaataccgaaagcatgagcaaagcaattcagcagtataccgttgatgcacgtctgcatgccgttttcgaacagagcggtgaaagcggtaaaagctttgattatagccagagcctgaaaaccaccacctatggtagcagcgtgccggaacagcagattaccgcatatctgagccgtattcagcgtggtggctacattcagccgtttggctgcatgatcgcagttgatgaaagcagctttcgcattattggctacagcgaaaatgcacgtgaaatgctgggcattatgccgcagagcgttccgaccctggaaaaaccggaaattctggcaatgggcaccgatgttcgtagcctgtttaccagcagcagctccattctgctggaacgtgcctttgttgcccgtgaaattaccctgctgaatccggtttggattcatagcaaaaacaccggcaaaccgttttatgcaattctgcatcgtattgatgttggcgtggttattgatctggaaccggcacgtaccgaagatccggcactgagcattgccggtgcagttcagagccagaaactggcagttcgtgcaattagccagctgcaggcactgcctggtggtgatatcaaactgctgtgtgataccgttgttgaaagcgttcgtgatctgaccggctacgaccgtgttatggtgtataaattccacgaagatgaacatggtgaagttgttgcagaaagcaaacgtgatgacctggaaccgtatattggtctgcattatccagcaaccgatattccgcaggcaagccgtttcctgttcaaacagaatcgtgtgcgcatgattgttgattgtaatgcaacaccggttctggttgttcaggatgatcgtctgacccagagcatgtgtctggttggtagcaccctgcgtgcaccgcatggttgtcatagccagtatatggcaaatatgggtagcatcgcaagcctggccatggcggtgatcatcaatggtaatgaagatgatggtagcaatgttgcaagcggtcgtagcagcatgcgtctgtggggtctggttgtgtgtcatcataccagcagtcgctgcattccgtttccgctgcgttatgcatgtgaatttctgatgcaggcatttggactgcagctgaatatggaactgcaactggcactgcagatgagcgaaaaacgtgttctgcgtacccagaccctgctgtgcgatatgctgctgcgtgatagtccggcaggcattgttacccagagcccgagcattatggatctggtgaaatgcgatggtgcagcctttctgtatcacggtaaatactatccgctgggtgttgcaccgagcgaagttcagattaaagatgttgttgagtggctgctggcaaatcatgcagatagcaccggtctgagcaccgatagcctgggtgatgcaggttatccgggtgcagcagcactgggagatgcagtttgtggtatggcagttgcatacattaccaaacgcgattttctgttttggtttcgtagccataccgccaaagaaatcaaatggggtggtgcaaaacatcacccggaagataaagatgacggtcagcgtatgcatccgcgtagtagctttcaggcatttctggaagtggtgaaaagccgtagccagccgtgggaaaccgcagaaatggatgcaattcatagcctgcaactgattctgcgcgatagcttcaaagaaagcgaagcagcaatgaatagcaaagttgttgatggtgttgttcagccgtgtcgtgatatggcaggcgaacagggtattgatgaactgggtgcaggttctggtagcggcagcggttctgctagcgccggcgacctgggcaagaagctgctggaagccgccagagccggacaggacgacgaagtgcggatcctgatggccaacggcgccgacgtgaacgccgacgatacctggggctggacccctctgcacctggccgcctatcagggccacctggaaatcgtggaagtgctgctgaagaacggggccgatgtgaatgcctacgactacatcggctggacacccctgcatctcgctgccgacggccacctcgagattgtcgaggtcctgctgaaaaatggcgctgatgtgaacgccagcgattatatcggcgacacccccctccatctggccgcccacaacggccatctggaaattgtcgaagtcctcctcaagcacggcgcagatgtcaacgcccaggacaagttcggcaagaccgccttcgacatcagcatcgacaacggcaacgaggacctggccgagatcctccagcatcatcaccatcaccattaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgagtgtcaacttagcttcccagttgcgggaagggacgaaaaaatcccactccatggcggagaacgtcggctttgtcaaatgcttcctcaagggcgttgtcgagaaaaattcctaccgtaagctggttggcaatctctactttgtctacagtgccatggaagaggaaatggcaaaatttaaggaccatcccatcctcagccacatttacttccccgaactcaaccgcaaacaaagcctagagcaagacctgcaattctattacggctccaactggcggcaagaagtgaaaatttctgccgctggccaagcctatgtggaccgagtccggcaagtggccgctacggcccctgaattgttggtggcccattcctacacccgttacctgggggatctttccggcggtcaaattctcaagaaaattgcccaaaatgccatgaatctccacgatggtggcacagctttctatgaatttgccgacattgatgacgaaaaggcttttaaaaatacctaccgtcaagctatgaatgatctgcccattgaccaagccaccgccgaacggattgtggatgaagccaatgacgcctttgccatgaacatgaaaatgttcaacgaacttgaaggcaacctgatcaaggcgatcggcattatggtgttcaacagcctcacccgtcgccgcagtcaaggcagcaccgaagttggcctcgccacctccgaaggctagaagcttggatccattaaagaggagaaacccgggcatggccgtcactgatttaagtttgaccaattcttccctgatgcctacgttgaacccgatgattcaacagttggccctggcgatcgccgctagttggcaaagtttacccctcaagccctatcaattgccggaggatttgggctacgtagaaggccgcctggaaggggaaaagttagtgattgaaaatcggtgctaccaaacgccccagtttcgcaaaatgcatttggagttggccaaggtgggcaaagggttggatattctccactgtgtaatgtttcctgagcctttatacggtctacctttgtttggctgtgacattgtggccggccccggtggagtaagtgcggctattgcggatctatcccccacccaaagcgatcgccaattgcccgcagcgtaccaaaaatcattggcagagctaggccagccagaatttgagcaacaacgggaattgcccccctggggagaaatattttctgaatattgtttattcatccgtcccagcaatgtcactgaagaagaaagatttgtacaaagggtagtggactttttgcaaattcattgtcaccaatccatcgttgccgaacccttgtctgaagctcaaactttggagcaccgtcaggggcaaattcattactgccaacaacaacagaaaaatgataaaacccgtcgggtactggaaaaagcttttggggaagcttgggcggaacggtatatgagccaagtcttatttgatgttatccaataagacgtcggtaccctcgagtctggtaaagaaaccgctgctgcgaaatttgaacgcca

SEQ ID NO: 5 - DNA, 4292 bp, Artificial Sequence
  Plasmid pMH327
attaggaaattaatacgactcactataggggaattgtgagcggataacaattcccctgtagaaataattttgtttaactttaataaggagatataccatggttagcggtgttggtggtagcggtggtggtcgtggtggcggtcgcggaggtgaagaagaaccgagcagcagccataccccgaataatcgccgtggtggtgaacaggcacagagcagcggcaccaaaagcctgcgtccgcgtagcaataccgaaagcatgagcaaagcaattcagcagtataccgttgatgcacgtctgcatgccgttttcgaacagagcggtgaaagcggtaaaagctttgattatagccagagcctgaaaaccaccacctatggtagcagcgtgccggaacagcagattaccgcatatctgagccgtattcagcgtggtggctacattcagccgtttggctgcatgatcgcagttgatgaaagcagctttcgcattattggctacagcgaaaatgcacgtgaaatgctgggcattatgccgcagagcgttccgaccctggaaaaaccggaaattctggcaatgggcaccgatgttcgtagcctgtttaccagcagcagctccattctgctggaacgtgcctttgttgcccgtgaaattaccctgctgaatccggtttggattcatagcaaaaacaccggcaaaccgttttatgcaattctgcatcgtattgatgttggcgtggttattgatctggaaccggcacgtaccgaagatccggcactgagcattgccggtgcagttcagagccagaaactggcagttcgtgcaattagccagctgcaggcactgcctggtggtgatatcaaactgctgtgtgataccgttgttgaaagcgttcgtgatctgaccggctacgaccgtgttatggtgtataaattccacgaagatgaacatggtgaagttgttgcagaaagcaaacgtgatgacctggaaccgtatattggtctgcattatccagcaaccgatattccgcaggcaagccgtttcctgttcaaacagaatcgtgtgcgcatgattgttgattgtaatgcaacaccggttctggttgttcaggatgatcgtctgacccagagcatgtgtctggttggtagcaccctgcgtgcaccgcatggttgtcatagccagtatatggcaaatatgggtagcatcgcaagcctggccatggcggtgatcatcaatggtaatgaagatgatggtagcaatgttgcaagcggtcgtagcagcatgcgtctgtggggtctggttgtgtgtcatcataccagcagtcgctgcattccgtttccgctgcgttatgcatgtgaatttctgatgcaggcatttggactgcagctgaatatggaactgcaactggcactgcagatgagcgaaaaacgtgttctgcgtacccagaccctgctgtgcgatatgctgctgcgtgatagtccggcaggcattgttacccagagcccgagcattatggatctggtgaaatgcgatggtgcagcctttctgtatcacggtaaatactatccgctgggtgttgcaccgagcgaagttcagattaaagatgttgttgagtggctgctggcaaatcatgcagatagcaccggtctgagcaccgatagcctgggtgatgcaggttatccgggtgcagcagcactgggagatgcagtttgtggtatggcagttgcatacattaccaaacgcgattttctgttttggtttcgtagccataccgccaaagaaatcaaatggggtggtgcaaaacatcacccggaagataaagatgacggtcagcgtatgcatccgcgtagtagctttcaggcatttctggaagtggtgaaaagccgtagccagccgtgggaaaccgcagaaatggatgcaattcatagcctgcaactgattctgcgcgatagcttcaaagaaagcgaagcagcaatgaatagcaaagttgttgatggtgttgttcagccgtgtcgtgatatggcaggcgaacagggtattgatgaactgggtgcaggttctggtagcggcagcggttctgctagcgccggcgatctgggtaaaaaactgctggaagcagcacgtgcaggtcaggatgatgaagttcgtattctggttgcaaatggtgcagatgtgaatgcctattttggcaccacaccgctgcatctggcagcagcacatggtcgtctggaaattgttgaagttctgctgaaaaacggtgccgatgttaatgcacaggatgtttggggtattaccccgttacacctggcagcatataacggtcatttagaaatcgtggaagtgctgcttaaatatggcgcagatgttaacgcccatgatacccgtggttggacccctctgcatttagcagcaattaatggtcacctggaaatcgtagaggtgcttctgaaaaatgtggccgatgtgaacgcgcaggatcgtagcggtaaaaccccgtttgatctggcaattgataatggcaatgaagatattgcagaggtgctgcagaaagcagcaaaactgaatcatcatcaccatcaccattaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgagtgtcaacttagcttcccagttgcgggaagggacgaaaaaatcccactccatggcggagaacgtcggctttgtcaaatgcttcctcaagggcgttgtcgagaaaaattcctaccgtaagctggttggcaatctctactttgtctacagtgccatggaagaggaaatggcaaaatttaaggaccatcccatcctcagccacatttacttccccgaactcaaccgcaaacaaagcctagagcaagacctgcaattctattacggctccaactggcggcaagaagtgaaaatttctgccgctggccaagcctatgtggaccgagtccggcaagtggccgctacggcccctgaattgttggtggcccattcctacacccgttacctgggggatctttccggcggtcaaattctcaagaaaattgcccaaaatgccatgaatctccacgatggtggcacagctttctatgaatttgccgacattgatgacgaaaaggcttttaaaaatacctaccgtcaagctatgaatgatctgcccattgaccaagccaccgccgaacggattgtggatgaagccaatgacgcctttgccatgaacatgaaaatgttcaacgaacttgaaggcaacctgatcaaggcgatcggcattatggtgttcaacagcctcacccgtcgccgcagtcaaggcagcaccgaagttggcctcgccacctccgaaggctagaagcttggatccattaaagaggagaaacccgggcatggccgtcactgatttaagtttgaccaattcttccctgatgcctacgttgaacccgatgattcaacagttggccctggcgatcgccgctagttggcaaagtttacccctcaagccctatcaattgccggaggatttgggctacgtagaaggccgcctggaaggggaaaagttagtgattgaaaatcggtgctaccaaacgccccagtttcgcaaaatgcatttggagttggccaaggtgggcaaagggttggatattctccactgtgtaatgtttcctgagcctttatacggtctacctttgtttggctgtgacattgtggccggccccggtggagtaagtgcggctattgcggatctatcccccacccaaagcgatcgccaattgcccgcagcgtaccaaaaatcattggcagagctaggccagccagaatttgagcaacaacgggaattgcccccctggggagaaatattttctgaatattgtttattcatccgtcccagcaatgtcactgaagaagaaagatttgtacaaagggtagtggactttttgcaaattcattgtcaccaatccatcgttgccgaacccttgtctgaagctcaaactttggagcaccgtcaggggcaaattcattactgccaacaacaacagaaaaatgataaaacccgtcgggtactggaaaaagcttttggggaagcttgggcggaacggtatatgagccaagtcttatttgatgttatccaataagacgtcggtaccctcgagtctggtaaagaaaccgctgctgcgaaatttgaacgcca

SEQ ID NO: 6 - DNA, 4286 bp, Artificial Sequence
  Plasmid pMH328
attaggaaattaatacgactcactataggggaattgtgagcggataacaattcccctgtagaaataattttgtttaactttaataaggagatataccatggttagcggtgttggtggtagcggtggtggtcgtggtggcggtcgcggaggtgaagaagaaccgagcagcagccataccccgaataatcgccgtggtggtgaacaggcacagagcagcggcaccaaaagcctgcgtccgcgtagcaataccgaaagcatgagcaaagcaattcagcagtataccgttgatgcacgtctgcatgccgttttcgaacagagcggtgaaagcggtaaaagctttgattatagccagagcctgaaaaccaccacctatggtagcagcgtgccggaacagcagattaccgcatatctgagccgtattcagcgtggtggctacattcagccgtttggctgcatgatcgcagttgatgaaagcagctttcgcattattggctacagcgaaaatgcacgtgaaatgctgggcattatgccgcagagcgttccgaccctggaaaaaccggaaattctggcaatgggcaccgatgttcgtagcctgtttaccagcagcagctccattctgctggaacgtgcctttgttgcccgtgaaattaccctgctgaatccggtttggattcatagcaaaaacaccggcaaaccgttttatgcaattctgcatcgtattgatgttggcgtggttattgatctggaaccggcacgtaccgaagatccggcactgagcattgccggtgcagttcagagccagaaactggcagttcgtgcaattagccagctgcaggcactgcctggtggtgatatcaaactgctgtgtgataccgttgttgaaagcgttcgtgatctgaccggctacgaccgtgttatggtgtataaattccacgaagatgaacatggtgaagttgttgcagaaagcaaacgtgatgacctggaaccgtatattggtctgcattatccagcaaccgatattccgcaggcaagccgtttcctgttcaaacagaatcgtgtgcgcatgattgttgattgtaatgcaacaccggttctggttgttcaggatgatcgtctgacccagagcatgtgtctggttggtagcaccctgcgtgcaccgcatggttgtcatagccagtatatggcaaatatgggtagcatcgcaagcctggccatggcggtgatcatcaatggtaatgaagatgatggtagcaatgttgcaagcggtcgtagcagcatgcgtctgtggggtctggttgtgtgtcatcataccagcagtcgctgcattccgtttccgctgcgttatgcatgtgaatttctgatgcaggcatttggactgcagctgaatatggaactgcaactggcactgcagatgagcgaaaaacgtgttctgcgtacccagaccctgctgtgcgatatgctgctgcgtgatagtccggcaggcattgttacccagagcccgagcattatggatctggtgaaatgcgatggtgcagcctttctgtatcacggtaaatactatccgctgggtgttgcaccgagcgaagttcagattaaagatgttgttgagtggctgctggcaaatcatgcagatagcaccggtctgagcaccgatagcctgggtgatgcaggttatccgggtgcagcagcactgggagatgcagtttgtggtatggcagttgcatacattaccaaacgcgattttctgttttggtttcgtagccataccgccaaagaaatcaaatggggtggtgcaaaacatcacccggaagataaagatgacggtcagcgtatgcatccgcgtagtagctttcaggcatttctggaagtggtgaaaagccgtagccagccgtgggaaaccgcagaaatggatgcaattcatagcctgcaactgattctgcgcgatagcttcaaagaaagcgaagcagcaatgaatagcaaagttgttgatggtgttgttcagccgtgtcgtgatatggcaggcgaacagggtattgatgaactgggtgcaggttctggtagcggcagcggttctgctagcgccggcgatctgggtaaaaaactgctggaagcagcacgtgcaggtcaggatgatgaagttcgtattctgatggcaaatggtgcagatgttaatgcccatgatttttatggtattacaccgctgcatctggcagcaaattttggtcatctggaaattgttgaagtgctgctgaaacatggtgccgatgtgaacgcatttgattatgataataccccgttacacctggcagcagatgcaggccatttagaaatcgtggaagtgttactgaaatatggcgcagacgtgaatgcaagcgatcgtgatggtcatacccctctgcatttagcagcccgtgaaggccacctggaaatagtagaggttctgcttaaaaacggtgcggatgtaaatgcgcaggataaatttggtaaaaccgcctttgatatcagcatcgataatggcaatgaagatctggccgaaatcctgcagaaactgaatcatcatcaccatcaccattaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgagtgtcaacttagcttcccagttgcgggaagggacgaaaaaatcccactccatggcggagaacgtcggctttgtcaaatgcttcctcaagggcgttgtcgagaaaaattcctaccgtaagctggttggcaatctctactttgtctacagtgccatggaagaggaaatggcaaaatttaaggaccatcccatcctcagccacatttacttccccgaactcaaccgcaaacaaagcctagagcaagacctgcaattctattacggctccaactggcggcaagaagtgaaaatttctgccgctggccaagcctatgtggaccgagtccggcaagtggccgctacggcccctgaattgttggtggcccattcctacacccgttacctgggggatctttccggcggtcaaattctcaagaaaattgcccaaaatgccatgaatctccacgatggtggcacagctttctatgaatttgccgacattgatgacgaaaaggcttttaaaaatacctaccgtcaagctatgaatgatctgcccattgaccaagccaccgccgaacggattgtggatgaagccaatgacgcctttgccatgaacatgaaaatgttcaacgaacttgaaggcaacctgatcaaggcgatcggcattatggtgttcaacagcctcacccgtcgccgcagtcaaggcagcaccgaagttggcctcgccacctccgaaggctagaagcttggatccattaaagaggagaaacccgggcatggccgtcactgatttaagtttgaccaattcttccctgatgcctacgttgaacccgatgattcaacagttggccctggcgatcgccgctagttggcaaagtttacccctcaagccctatcaattgccggaggatttgggctacgtagaaggccgcctggaaggggaaaagttagtgattgaaaatcggtgctaccaaacgccccagtttcgcaaaatgcatttggagttggccaaggtgggcaaagggttggatattctccactgtgtaatgtttcctgagcctttatacggtctacctttgtttggctgtgacattgtggccggccccggtggagtaagtgcggctattgcggatctatcccccacccaaagcgatcgccaattgcccgcagcgtaccaaaaatcattggcagagctaggccagccagaatttgagcaacaacgggaattgcccccctggggagaaatattttctgaatattgtttattcatccgtcccagcaatgtcactgaagaagaaagatttgtacaaagggtagtggactttttgcaaattcattgtcaccaatccatcgttgccgaacccttgtctgaagctcaaactttggagcaccgtcaggggcaaattcattactgccaacaacaacagaaaaatgataaaacccgtcgggtactggaaaaagcttttggggaagcttgggcggaacggtatatgagccaagtcttatttgatgttatccaataagacgtcggtaccctcgagtctggtaaagaaaccgctgctgcgaaatttgaacgcca

SEQ ID NO: 7 - DNA, 4223 bp, Artificial Sequence
  Plasmid pMH329
attaggaaattaatacgactcactataggggaattgtgagcggataacaattcccctgtagaaataattttgtttaactttaataaggagatataccatggttagcggtgttggtggtagcggtggtggtcgtggtggcggtcgcggaggtgaagaagaaccgagcagcagccataccccgaataatcgccgtggtggtgaacaggcacagagcagcggcaccaaaagcctgcgtccgcgtagcaataccgaaagcatgagcaaagcaattcagcagtataccgttgatgcacgtctgcatgccgttttcgaacagagcggtgaaagcggtaaaagctttgattatagccagagcctgaaaaccaccacctatggtagcagcgtgccggaacagcagattaccgcatatctgagccgtattcagcgtggtggctacattcagccgtttggctgcatgatcgcagttgatgaaagcagctttcgcattattggctacagcgaaaatgcacgtgaaatgctgggcattatgccgcagagcgttccgaccctggaaaaaccggaaattctggcaatgggcaccgatgttcgtagcctgtttaccagcagcagctccattctgctggaacgtgcctttgttgcccgtgaaattaccctgctgaatccggtttggattcatagcaaaaacaccggcaaaccgttttatgcaattctgcatcgtattgatgttggcgtggttattgatctggaaccggcacgtaccgaagatccggcactgagcattgccggtgcagttcagagccagaaactggcagttcgtgcaattagccagctgcaggcactgcctggtggtgatatcaaactgctgtgtgataccgttgttgaaagcgttcgtgatctgaccggctacgaccgtgttatggtgtataaattccacgaagatgaacatggtgaagttgttgcagaaagcaaacgtgatgacctggaaccgtatattggtctgcattatccagcaaccgatattccgcaggcaagccgtttcctgttcaaacagaatcgtgtgcgcatgattgttgattgtaatgcaacaccggttctggttgttcaggatgatcgtctgacccagagcatgtgtctggttggtagcaccctgcgtgcaccgcatggttgtcatagccagtatatggcaaatatgggtagcatcgcaagcctggccatggcggtgatcatcaatggtaatgaagatgatggtagcaatgttgcaagcggtcgtagcagcatgcgtctgtggggtctggttgtgtgtcatcataccagcagtcgctgcattccgtttccgctgcgttatgcatgtgaatttctgatgcaggcatttggactgcagctgaatatggaactgcaactggcactgcagatgagcgaaaaacgtgttctgcgtacccagaccctgctgtgcgatatgctgctgcgtgatagtccggcaggcattgttacccagagcccgagcattatggatctggtgaaatgcgatggtgcagcctttctgtatcacggtaaatactatccgctgggtgttgcaccgagcgaagttcagattaaagatgttgttgagtggctgctggcaaatcatgcagatagcaccggtctgagcaccgatagcctgggtgatgcaggttatccgggtgcagcagcactgggagatgcagtttgtggtatggcagttgcatacattaccaaacgcgattttctgttttggtttcgtagccataccgccaaagaaatcaaatggggtggtgcaaaacatcacccggaagataaagatgacggtcagcgtatgcatccgcgtagtagctttcaggcatttctggaagtggtgaaaagccgtagccagccgtgggaaaccgcagaaatggatgcaattcatagcctgcaactgattctgcgcgatagcttcaaagaaagcgaagcagcaatgaatagcaaagttgttgatggtgttgttcagccgtgtcgtgatatggcaggcgaacagggtattgatgaactgggtgcaggttctggtagcggcagcggttctgctagcgccggccgtggtagccatcaccatcatcatcatggtagcgatctgggtaaaaaactgctggaagcagcacgtgcaggtcaggatgatgaagttcgtattctgatggcaaatggtgcagatgttaatgccatggatattctgggtctgacaccgctgcatctggcagcagttgccggtcatctggaaattgttgaagttctgctgaaaaatagcgctgatgtgaatgcgattgatgaagatggcgaaacccctctgcatctgagcgcagcaatgggccatctgaaaatcgtggaagtgctgttaaaacatggtgccgatgtgaacgcacaggataaatttggtaaaaccgcctttgatatcagcatcgattatggcaatgaagatctggcagaaatcctgcagaaactgaatcatcatcaccatcaccattaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgagtgtcaacttagcttcccagttgcgggaagggacgaaaaaatcccactccatggcggagaacgtcggctttgtcaaatgcttcctcaagggcgttgtcgagaaaaattcctaccgtaagctggttggcaatctctactttgtctacagtgccatggaagaggaaatggcaaaatttaaggaccatcccatcctcagccacatttacttccccgaactcaaccgcaaacaaagcctagagcaagacctgcaattctattacggctccaactggcggcaagaagtgaaaatttctgccgctggccaagcctatgtggaccgagtccggcaagtggccgctacggcccctgaattgttggtggcccattcctacacccgttacctgggggatctttccggcggtcaaattctcaagaaaattgcccaaaatgccatgaatctccacgatggtggcacagctttctatgaatttgccgacattgatgacgaaaaggcttttaaaaatacctaccgtcaagctatgaatgatctgcccattgaccaagccaccgccgaacggattgtggatgaagccaatgacgcctttgccatgaacatgaaaatgttcaacgaacttgaaggcaacctgatcaaggcgatcggcattatggtgttcaacagcctcacccgtcgccgcagtcaaggcagcaccgaagttggcctcgccacctccgaaggctagaagcttggatccattaaagaggagaaacccgggcatggccgtcactgatttaagtttgaccaattcttccctgatgcctacgttgaacccgatgattcaacagttggccctggcgatcgccgctagttggcaaagtttacccctcaagccctatcaattgccggaggatttgggctacgtagaaggccgcctggaaggggaaaagttagtgattgaaaatcggtgctaccaaacgccccagtttcgcaaaatgcatttggagttggccaaggtgggcaaagggttggatattctccactgtgtaatgtttcctgagcctttatacggtctacctttgtttggctgtgacattgtggccggccccggtggagtaagtgcggctattgcggatctatcccccacccaaagcgatcgccaattgcccgcagcgtaccaaaaatcattggcagagctaggccagccagaatttgagcaacaacgggaattgcccccctggggagaaatattttctgaatattgtttattcatccgtcccagcaatgtcactgaagaagaaagatttgtacaaagggtagtggactttttgcaaattcattgtcaccaatccatcgttgccgaacccttgtctgaagctcaaactttggagcaccgtcaggggcaaattcattactgccaacaacaacagaaaaatgataaaacccgtcgggtactggaaaaagcttttggggaagcttgggcggaacggtatatgagccaagtcttatttgatgttatccaataagacgtcggtaccctcgagtctggtaaagaaaccgctgctgcgaaatttgaacgcca

SEQ ID NO: 8 - DNA, 2120 bp, Artificial Sequence
  Plasmid pMH303
ggtcgccaccatgatgttccttcctactgattattgttgcagactgagcgaccaggaatacatggaactcgtcttcgagaacggacagatactcgcaaaaggccagaggtcaaatgttagtctccataatcagcggacgaaaagcatcatggatctgtatgaggccgaatacaacgaagattttatgaaaagtattatccatggagggggtggcgctattaccaacctgggagatacccaagtggtcccacagtcccacgtagcagccgctcacgagaccaatatgctggagtccaacaaacacgtagacacgcgtgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcaggcaacgcacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaagatctcgagc

SEQ ID NO: 9 - DNA, 1382 bp, Artificial Sequence
  Plasmid pOSY115
tctagagctagcgaattcgaagccaccatggccgtcatggcgccccgaaccctcctcctgctactctcgggggccctggccctgacccagacctgggcgggaggaagcggcgtgaccacactgtccggactttctggtgagcagggaccatccggcgacatgaccacagaagaggattctgccacacacatcaagttcagcaaacgcgacgaagacggtagagagttggcaggggcaaccatggaactgcgcgatagctccggcaaaaccataagcacatggatctccgacggccatgttaaggatttctacctgtatcccggcaagtacacctttgtggagacagcagcaccagacggatatgaggttgcaacccctatagaatttacagtgaacgaggacggacaggttaccgtggatggagaggcaacagaaggcgatgcacacaccggcggttctggatacccatacgatgttccagattacgctggcggttctggactgtcttcccagcccaccatccccatcgtgggcatcattgctggcctggttctgcttggagctgtgatcactggagctgtggtcgctgctgtgatgtggcggcgcaagagctcaggcggttctggaggcggttctggagtgagcaagggcgaggagctgttcaccggggtggtgcccatcctggtcgagctggacggcgacgtaaacggccacaagttcagcgtgagaggcgagggcgagggcgatgccaccaacggcaagctgaccctgaagttcatcagtactaccggcaagctgcccgtgccctggcccacccttgtgaccaccctgagccacggcgtgcaggtgttcgcccgctaccccgaccacatgaagcagcacgacttcttcaagtccgctatgcccgaaggctacgtccaggagcgcaccatcttcttcaaggacgacggcacctacaagacccgcgccgaggtgaagttcgagggcgacaccctggtgaaccgcatcgagctgaagggcgtcgatttcaaggaggacggcaacatcctggggcacaagctggagtacaacttcaacagccacaacatctatataatggccgtcaagcagaagaacggcatcaaggccaacttcaagatccgccacaacgtggaggacggcagcgtgcagctcgccgaccactaccagcagaacacccccatcggcgacggccccgtgctgctgcccgacagccactacctgagcacccagtccaagcttagcaaagaccccaacgagaagcgcgatcacatggtcctgctggagttccgcaccgccgccgggatcactctcggcatggacgagctgtacaagtgaggatccgcggccgcgtcgacaatcaa

SEQ ID NO: 10 - DNA, 4574 bp, Artificial Sequence
  Plasmid pOSY117
attaggaaattaatacgactcactataggggaattgtgagcggataacaattcccctgtagaaataattttgtttaactttaataaggagatataccatggttagcggtgttggtggtagcggtggtggtcgtggtggcggtcgcggaggtgaagaagaaccgagcagcagccataccccgaataatcgccgtggtggtgaacaggcacagagcagcggcaccaaaagcctgcgtccgcgtagcaataccgaaagcatgagcaaagcaattcagcagtataccgttgatgcacgtctgcatgccgttttcgaacagagcggtgaaagcggtaaaagctttgattatagccagagcctgaaaaccaccacctatggtagcagcgtgccggaacagcagattaccgcatatctgagccgtattcagcgtggtggctacattcagccgtttggctgcatgatcgcagttgatgaaagcagctttcgcattattggctacagcgaaaatgcacgtgaaatgctgggcattatgccgcagagcgttccgaccctggaaaaaccggaaattctggcaatgggcaccgatgttcgtagcctgtttaccagcagcagctccattctgctggaacgtgcctttgttgcccgtgaaattaccctgctgaatccggtttggattcatagcaaaaacaccggcaaaccgttttatgcaattctgcatcgtattgatgttggcgtggttattgatctggaaccggcacgtaccgaagatccggcactgagcattgccggtgcagttcagagccagaaactggcagttcgtgcaattagccagctgcaggcactgcctggtggtgatatcaaactgctgtgtgataccgttgttgaaagcgttcgtgatctgaccggctacgaccgtgttatggtgtataaattccacgaagatgaacatggtgaagttgttgcagaaagcaaacgtgatgacctggaaccgtatattggtctgcattatccagcaaccgatattccgcaggcaagccgtttcctgttcaaacagaatcgtgtgcgcatgattgttgattgtaatgcaacaccggttctggttgttcaggatgatcgtctgacccagagcatgtgtctggttggtagcaccctgcgtgcaccgcatggttgtcatagccagtatatggcaaatatgggtagcatcgcaagcctggccatggcggtgatcatcaatggtaatgaagatgatggtagcaatgttgcaagcggtcgtagcagcatgcgtctgtggggtctggttgtgtgtcatcataccagcagtcgctgcattccgtttccgctgcgttatgcatgtgaatttctgatgcaggcatttggactgcagctgaatatggaactgcaactggcactgcagatgagcgaaaaacgtgttctgcgtacccagaccctgctgtgcgatatgctgctgcgtgatagtccggcaggcattgttacccagagcccgagcattatggatctggtgaaatgcgatggtgcagcctttctgtatcacggtaaatactatccgctgggtgttgcaccgagcgaagttcagattaaagatgttgttgagtggctgctggcaaatcatgcagatagcaccggtctgagcaccgatagcctgggtgatgcaggttatccgggtgcagcagcactgggagatgcagtttgtggtatggcagttgcatacattaccaaacgcgattttctgttttggtttcgtagccataccgccaaagaaatcaaatggggtggtgcaaaacatcacccggaagataaagatgacggtcagcgtatgcatccgcgtagtagctttcaggcatttctggaagtggtgaaaagccgtagccagccgtgggaaaccgcagaaatggatgcaattcatagcctgcaactgattctgcgcgatagcttcaaagaaagcgaagcagcaatgaatagcaaagttgttgatggtgttgttcagccgtgtcgtgatatggcaggcgaacagggtattgatgaactgggtgcaggttctggtagcatggtgtccaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggatcaggtcgtggcgtgcctcatatcgtgatggtggacgcctacaagcgttacaagggcggttctggacatcatcaccatcaccattaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgagtgtcaacttagcttcccagttgcgggaagggacgaaaaaatcccactccatggcggagaacgtcggctttgtcaaatgcttcctcaagggcgttgtcgagaaaaattcctaccgtaagctggttggcaatctctactttgtctacagtgccatggaagaggaaatggcaaaatttaaggaccatcccatcctcagccacatttacttccccgaactcaaccgcaaacaaagcctagagcaagacctgcaattctattacggctccaactggcggcaagaagtgaaaatttctgccgctggccaagcctatgtggaccgagtccggcaagtggccgctacggcccctgaattgttggtggcccattcctacacccgttacctgggggatctttccggcggtcaaattctcaagaaaattgcccaaaatgccatgaatctccacgatggtggcacagctttctatgaatttgccgacattgatgacgaaaaggcttttaaaaatacctaccgtcaagctatgaatgatctgcccattgaccaagccaccgccgaacggattgtggatgaagccaatgacgcctttgccatgaacatgaaaatgttcaacgaacttgaaggcaacctgatcaaggcgatcggcattatggtgttcaacagcctcacccgtcgccgcagtcaaggcagcaccgaagttggcctcgccacctccgaaggctagaagcttggatccattaaagaggagaaacccgggcatggccgtcactgatttaagtttgaccaattcttccctgatgcctacgttgaacccgatgattcaacagttggccctggcgatcgccgctagttggcaaagtttacccctcaagccctatcaattgccggaggatttgggctacgtagaaggccgcctggaaggggaaaagttagtgattgaaaatcggtgctaccaaacgccccagtttcgcaaaatgcatttggagttggccaaggtgggcaaagggttggatattctccactgtgtaatgtttcctgagcctttatacggtctacctttgtttggctgtgacattgtggccggccccggtggagtaagtgcggctattgcggatctatcccccacccaaagcgatcgccaattgcccgcagcgtaccaaaaatcattggcagagctaggccagccagaatttgagcaacaacgggaattgcccccctggggagaaatattttctgaatattgtttattcatccgtcccagcaatgtcactgaagaagaaagatttgtacaaagggtagtggactttttgcaaattcattgtcaccaatccatcgttgccgaacccttgtctgaagctcaaactttggagcaccgtcaggggcaaattcattactgccaacaacaacagaaaaatgataaaacccgtcgggtactggaaaaagcttttggggaagcttgggcggaacggtatatgagccaagtcttatttgatgttatccaataagacgtcggtaccctcgagtctggtaaagaaaccgctgctgcgaaatttgaacgcca

(19) United States
(12) Patent Application Publication
(10) Pub. No.: US 2024/0060086 A1
(43) Pub. Date: Feb. 22, 2024

(54) LIGHT-CONTROLLED VIRAL TRANSDUCTION

(71) Applicant: ALBERT-LUDWIGS-UNIVERSITÄT FREIBURG, München (DE)

(72) Inventors: Maximilian HÖRNER, Freiburg (DE); Wilfried WEBER, Freiburg (DE)

(73) Assignee: ALBERT-LUDWIGS-UNIVERSITÄT FREIBURG, München (DE)

(21) Appl. No.: 18/037,945

(22) PCT Filed: Nov. 22, 2021
(86) PCT No.: PCT/EP2021/082518

§ 371 Date: May 19, 2023

(30) Foreign Application Priority Data
Nov. 20, 2020 (EP) 20208920.7

(51) Int. Cl. C12N 15/86; C12N 7/00; C12N 15/63

(52) U.S. Cl. CPC C12N 15/86; C12N 7/00; C12N 15/635; C12N 2750/14122; C12N 2750/14143; C12N 2830/002; C12N 2750/14145

(57) ABSTRACT

The present invention is directed to a kit of parts having biological activity, the kit of parts comprising a first target comprising a first protein and a second target comprising a second protein, wherein the first protein and the second protein are suitable to form a heterodimer upon irradiation with UV, visible or infrared light in a first wavelength range or in the dark, which can be reversed upon irradiating the heterodimer with UV, visible or infrared light in a second wavelength range or in the dark, wherein the second wavelength range is different from the first wavelength range, wherein the biological activity consists of triggering both the uptake of DNA, RNA, proteins, or small molecules into a cell which is preferably genetically unmodified, and biological effects, and characterized in that at least one of the first and the second target itself has reduced biological activity as compared with the heterodimer.

### BACKGROUND OF THE INVENTION

Single-cell sequencing and, more recently, single-cell multimodal omics approaches are opening up new avenues for biotechnological and medical uses. In order exploit these new possibilities in multicellular systems, there is a need for developing technologies for engineering mammalian cells at single-cell scale. However, current technologies for single-cell genetic engineering such as microinjection, single-cell electroporation or optical transfection based on laser-induced cell membrane perforation are not widely applicable due to high invasiveness, low throughput and the requirement for complicated instrumentation. Although optogenetics can be used to optically control processes in single cells, it requires the previous transfer of genetic information into the collective of cells to render them light responsive. This prevents or complicates the use of primary cells and tissues and may affect surrounding off-target cells by pleiotropic side effects. The present invention overcomes these problems and limitations in a surprising way.

### DISCLOSURE OF INVENTION

### Technical Problem

Hence, it is an object of the present invention to overcome the above-mentioned disadvantages.

In particular, an object of the present invention is to allow single-cell (preferably genetic) engineering without using microinjection, single-cell electroporation or optical transfection based on laser-induced cell membrane perforation.

Another object of the present invention is to provide technologies for engineering genetically unmodified cells at single-cell scale.

It is an object of the present invention to provide single-cell (preferably genetic) engineering which can be initiated by low doses of cell friendly light, preferably not UV. Preferably, the initiation of single-cell engineering is commensurate with the illumination.

It is another object of the present invention to provide single-cell (preferably genetic) engineering which does not require active internalization of cell surface structures that are targeted for single-cell engineering.

It is yet another object of the present invention to provide single-cell (preferably genetic) engineering which does not affect cell surface structures that are targeted for single-cell engineering in a such way that biological effects such as receptor activation and/or signal transduction are initiated as a result of targeting the single cell.

It is a further object of the present invention to provide a kit of parts for single-cell (preferably genetic) engineering which, once activated, can also be de-activated by low doses of cell friendly light, preferably not UV.

It is an object of the present invention to provide a tool for locally engineering cell clusters by using locally restricted beams of low doses of cell friendly light, preferably not UV, while sparing the surrounding cells.

It is an object of the present invention to provide (preferably genetic) engineering that can be modulated over time by the simple illumination with a selected wavelength range in the red or near-infrared range.

It is a further object of the present invention to provide fast cell engineering, preferably genetic cell engineering.

It is an object of the present invention to provide a tissue which can contain engineered and non-engineered cells.

It is a further object of the present invention to provide (preferably genetic) cell engineering with improved accuracy.

It is a further object of the present invention to provide (preferably genetic) cell engineering that is stable, e.g. is not neutralized.

### Solution to Problem

All these objects are surprisingly solved by the subject matter of the independent claims.

The present invention overcomes the above-identified limitations by optically guiding the selective transfer of molecules, e.g. genetic information, into single target cells. The present invention (i) does not require the previous genetic engineering of target cells, (ii) relies on standard hardware for optical stimulation (e.g. light-emitting diodes or a conventional confocal microscope), (iii) is compatible with immortalized and primary cells, and (iv) relies on non-invasive light. (v) As transfer (e.g. gene delivery) is controlled at the level of (e.g. viral) cell entry, it minimizes the impact on off-target neighbouring cells. One embodiment of the invention relates to OptoAAV which is based on adeno-associated viral (AAV) vectors that play a key role as gene delivery vehicle in fundamental research and clinically licensed gene therapies. AAV vectors are non-enveloped, single-stranded DNA vectors that transduce both dividing and non-dividing cells and provide an excellent safety profile due to the absence of AAV-associated pathologies and episomal persistence. Thus, the OptoAAV technology preferably comprises an engineered adeno-associated viral (AAV) vector system (AAV-2) and a light-responsive adapter protein (e.g. PhyB) that mediates selective interaction of the AAV with a target. For example, the viral vector is genetically modified to be blind to its natural cellular receptor heparan sulfate proteoglycan (HSPG) and to expose the phytochrome-interacting factor 6 (PIF6; amino acids 1-100) on the capsid surface.

### Advantageous Effects of Invention

An advantage of the present invention is that genetically unmodified cells can be used. Of course, the present invention also works with genetically modified cells.

Since kits of parts according to various exemplary embodiments of the present invention comprise a first target comprising a first protein and a second target comprising a second protein, wherein the first protein and the second protein reversibly form a heterodimer upon irradiation (e.g. with visible or infrared light in a specific wavelength range) or in the dark, the kit of parts can be readily activated, thus exhibiting biological activity.

Further, since the heterodimer formation is reversible (using e.g. visible or infrared light in a specific wavelength range, which is different from the wavelength range for heterodimer formation or in the dark), the kit of parts can also be readily de-activated.

Using two separate targets comprising two different proteins which can form a heterodimer ensures that biological activity is controlled and not the result of chance events. The efficiency of cell engineering (e.g. transduction efficiency) by the present invention preferably is at least 50%, more preferably at least 55%, even more preferably at least 60%. If cell engineering relies on transduction of genes encoding fluorescent proteins, transduction efficiency can e.g. be measured by measuring fluorescent cells.

Since biological activity can essentially only occur by irradiating the kit of parts with UV, visible or infrared light in a specific wavelength range or in the dark, the biological activity is controllable by controlling irradiation.

Since heterodimer formation occurs under mild conditions, living cells are not negatively affected by the kit of parts according to the present invention. In particular, the present invention does not require and can be carried out without active internalization of cell surface structures that are targeted by the heterodimer. Of course, the invention does not generally exclude internalization (e.g. for liposomes or specific viral vectors). Moreover, the present invention does not require and can be carried out without affecting cell surface structures that are targeted by the heterodimer in such way that biological effects such as receptor activation and/or signal transduction are initiated as a result of targeting. Of course, the invention does not generally exclude receptor activation and/or signal transduction.

Further, since the kit of parts can be de-activated quickly and under mild conditions, cells can be easily engineered in a spatio-temporal manner.

Additionally, the kit of parts can be applied in regenerative medicine, tissue engineering, gene therapy or fundamental research.

Additionally, the kit of parts can be applied to organisms, including animals, particularly humans.

### BRIEF DESCRIPTION OF THE FIGURES

FIG. 1 shows the design and mode of function of the light-controlled viral transduction system.

FIG. 2 shows the characterization of OptoAAV system components.

FIG. 3 shows the characterization of the OptoAAV system.

FIG. 4 shows the modular adaptation of the OptoAAV system to different cell types.

FIG. 5 shows the spatiotemporally controlled transduction.

FIG. 6 shows the spatially resolved transduction of single cells using a conventional confocal microscope.

FIG. S1 shows the analysis of different PhyB-DARPin adapter proteins by SDS-PAGE after immobilized metal affinity chromatography (IMAC) purification.

FIG. S2 shows the spectral analysis of the different purified PhyB-DARPin adapter proteins.

FIG. S3 shows the light-dependent protein interaction between PhyB-DARPin_{EGFR} and PIF6.

FIG. S4 shows the binding of PhyB and PhyB-DARPin_{EGFR} to A-431 cells.

FIG. S5 shows the light-controlled recruitment of mVenus-PIF6 to the cell surface.

FIG. S6 shows the gating strategy of flow cytometry experiments analyzed in this study.

FIG. S7 shows the impact of heating of AAVs on transduction.

FIG. S8 shows the characterization of the OptoAAV system.

FIG. S9 shows the impact of the OptoAAV_{EGFR} system on EGFR activation.

FIG. S10 shows the light-controlled transduction of different cell lines with OptoAAV_{GFP} and the adapter protein PhyB-DARPin_{EGFR}.

FIG. S11 shows an analysis of the role of cellular target receptor internalization for the OptoAAV system.

FIG. S12 shows the spatiotemporally resolved transduction with one or two different transgenes.

FIG. S13 shows the light-controlled transduction of eFluor670-stained cells.

FIG. S14 shows the light-controlled transduction of CF SE-stained cells.

FIG. T1 shows an overview of light-controlled viral transduction systems

FIG. T2 shows nucleic acid sequences of plasmids generated in the example.

### DETAILED DESCRIPTION OF THE INVENTION

In one aspect, the present invention relates to a kit of parts having biological activity, the kit of parts comprising
a first target comprising a first protein,
a second target comprising a second protein,
   wherein the first protein and the second protein are suitable to form a heterodimer upon irradiation with UV, visible or infrared light in a first wavelength range or in the dark, which can be reversed upon irradiating the heterodimer with UV, visible or infrared light in a second wavelength range or in the dark, wherein the second wavelength range is different from the first wavelength range, characterized in that neither the first target itself not the second target itself has biological activity, but only the heterodimer.

In one embodiment, at least one of the first and the second target has reduced biological activity as compared with the heterodimer. The heterodimer is adapted to have biological activity upon exposure to at least one cell, preferably a genetically unmodified cell.

In an embodiment of the invention, visible light can be red, green or blue light, as defined below.

In a preferred embodiment, the first protein and the second protein are suitable to form a heterodimer upon irradiation with visible or infrared light in a first wavelength range, which can be reversed upon irradiating the heterodimer with visible or infrared light in a second wavelength range, wherein the second wavelength range is different from the first wavelength range. As an example, first and second proteins may be activated with light, but then return to the ground state on their own in the dark. Thus, heterodimers are obtained with light and lost in the dark. However, it is also within the scope of the present invention that irradiation with UV, visible or infrared light in a second wavelength range leads to loss (i.e. dissociation) of heterodimers.

It is important to note that the first and second protein can always be exchanged. If, for example, the first protein is PhyB and the second protein is PIF6, the present invention also relates to PIF6 being the first protein and PhyB being the second protein. Thus, PhyB can be either first or second protein and PIF6 either first or second protein. This applies to any combination of first and second proteins disclosed in this description, i.e. all of them can be exchanged and all exchanges are envisaged in individualized for. Thus, since PhyA/PIF3, PhyA/PIF6, PhyA/FHY1, PhyA/FHL, PhyB/PIF3, PhyB/PIF6, PhyB/FHY1, PhyB/FHL, BphP1/PpsR2 and BphP1/Q-PAS1 form heterodimers, it does not matter for the present invention which protein of the heterodimer-forming pair is the first protein and which one is the second protein. This applies mutatis mutandis to all protein pairs disclosed herein.

Since it does not matter for the present invention which protein of the heterodimer-forming pair is the first protein and which one is the second protein, exchanging those proteins is within the scope of the present invention and, in case of an exchange, the statements that follow apply in analogy to the exchanged entity.

Light and Dark

“Visible light” as used herein excludes light having a wavelength smaller than 400 nm (i.e. UV light). Visible light has a wavelength in the range of 400 to less than 700 nm. Blue light has a wavelength in the range of 400 to less than 500 nm, green light has a wavelength in the range of 500 to less than 580 nm and red light has a wavelength in the range of 580 to less than 700 nm. “Infrared light” as used herein refers to light having a wavelength in the range of 700-1000 nm. “Near-infrared light” as used herein refers to light in a wavelength range of 700-790 nm.

An exemplary kit of parts of the present invention may comprise a first target which comprises a first protein (here: PhyB), and a virus or derivative thereof as the second target which comprises a second protein (here: PIF6). When a mixture of first and second target is irradiated with visible light with a wavelength of about 660 nm, PhyB is activated and the activated PhyB interacts with PIF6 to form a heterodimer. By the heterodimerization, contacts (e.g. bonds or cross-links) are formed between the first and second targets, leading to the formation of a heterodimer. The heterodimer can then be separated by irradiating it with infrared light with a wavelength of about 740 nm. Hereby, the activated state of PhyB is reversed. The ground state of PhyB cannot interact with PIF6, so that heterodimerization is reversed, the contacts/links between the first and second targets are thus separated. Hence, (at least a remainder of) the kit of parts is re-obtained which can be used again by irradiating it with visible light with a wavelength of about 660 nm. Heterodimer formation is thus a reversible process in the present invention.

This reversibility has the advantage that the kit of parts can be used in different steps or rounds. For example, a different second target can be used after the first-step or first-round second target (e.g. AAV with a different transgene). This allows to engineer (e.g. transduce) different cells with different transgenes (e.g. GFP and mScarlet), which is illustrated as an example in FIG. 5B. It shows the spatiotemporally resolved transduction with two different transgenes. A-431 cells were transduced spatially resolved, using a photomask 1 and OptoAAV_{GFP}. Following an incubation in the dark and washing with PBS, the same procedure was repeated using a photomask 2 and OptoAAVm_{Scarlet}. After the second 2 h incubation step in the dark, cells were washed with PBS and incubated for 44 h in cell culture medium under 740 nm illumination. Of course, this is just an illustrative example and the invention could also be carried out with other transgenes such as two different therapeutic transgenes.

If the activation of the targets in the claimed kit of parts was not reversible, the second-step or second-round second target would also bind to first targets activated in the first step or round since they might not all be used up by second targets in the first step or round. Thus, the reversibility is advantageous.

In the above example, the first target may comprise a receptor protein agonist or antagonist which can bind to a cell surface. The first target may thus comprise a receptor protein agonist or antagonist and PhyB. When put in contact with a cell, the first target binds to a cell surface. Upon irradiation with visible light with a wavelength of about 660 nm, PhyB in the first target is activated and the activated PhyB interacts with PIF6 in the second target to form a heterodimer. This heterodimerization at the cell surface leads to biological activity, as described below. However, that biological activity does not require active internalization of the receptor protein that is targeted by the first target (and thus the heterodimer). Moreover, said biological activity does not require that the receptor protein be activated (e.g. by receptor protein clustering) and/or that signal transduction be initiated at the receptor protein.

However, the invention does not generally exclude active receptor internalization and/or activation. Thus, the receptor protein that is targeted by the first target (and thus the heterodimer) may be activated in some embodiments of the invention. Moreover, the receptor protein may be activated (e.g. by receptor protein clustering) and/or that signal transduction may be initiated at the receptor protein in some embodiments of the invention.

Biological Activity

Biological activity within the meaning of this invention consists of triggering both the uptake of the heterodimer (preferably without required active internalization of the targeted cell surface structure such as the targeted receptor protein), including DNA, or RNA, proteins, or small molecules comprised therein, into a cell, preferably a genetically unmodified cell, and biological effects in the preferably genetically unmodified cell, wherein these biological effects are different from effects that are (usually) caused by activating the targeted cell surface structure such as activation of the targeted receptor protein and subsequent signal transduction. By contrast, the biological effects of the present invention are caused by DNA, or RNA, proteins, or small molecules provided by the heterodimer and preferably comprise signal transduction; metabolic processes such as glycolysis, beta-oxidation, glycolysis, metabolism of amino acids; anabolic processes such as gluconeogenesis, glycogen synthesis, lipid synthesis, gene expression, gene silencing, DNA synthesis, RNA synthesis, preferably gene transcription, protein synthesis, peptide synthesis, post-translational modifications, preferably glycosylation, phosphorylation, ubiquitination, sumoylation, methylation or acetylation; cell growth; cell division; cell proliferation; cell survival; cell differentiation; cell ageing; organelle growth; organelle proliferation; polymerization or depolymerization of the cytoskeleton; viral replication; reverse transcription; phosphorylation of nucleosides; phosphorylation of nucleotides; apoptosis, or necrosis.

In a particularly preferred embodiment, the biological activity consists of triggering both the uptake of DNA, RNA, proteins, or small molecules into a cell which is preferably genetically unmodified, and biological effects as defined hereinbefore.

The second target itself has reduced biological activity as compared with the heterodimer. Reduced biological activity means a reduction of at least 50%, preferably at least 70%, more preferably at least 90%, most preferably at least 95%.

The first target itself may have no or neglectable biological activity. Nevertheless, it may be taken up by a cell, e.g. a genetically unmodified cell. However, the mere uptake and, if applicable, unspecific follow-up processes such as proteolysis and/or antigen presentation do not qualify as biological activity within the meaning of this invention.

The genetically unmodified cell is preferably living and untreated, wherein treatment comprises any step that is suitable to induce biological activity as defined hereinbefore. The treatment that is suitable to induce biological activity may include using contacting the cell with a virus or viral vector, microinjection, single-cell electroporation, laser treatment e.g. optical transfection based on laser-induced cell membrane perforation, microinjection, exposure to a detergent (preferably SDS, digitonin, dodecyl laureate, n-dodecyl β-D-maltoside, Triton X-100), a transfection reagent, a ionophore, high or low salt concentrations (preferably salt concentrations below 250 mOsm/1 and above 350 mOsm/1), high or low temperatures (preferably below 25° C. and above 39° C.), high or low pH (preferably below pH 6.0 and above pH 8.0).

Specifically, the cell may preferably but without limitation be one or more selected from the group consisting of a bacterial cell, a fungal cell, an algae, a plant cell, a protoplast, an animal cell, preferably a mammalian cells, in particular human-derived stem cells, muscle-derived stem cells, dental pulp stem cells, nasal concha-derived mesenchymal stromal cells, an osteoblast, a myoblast, a tenocyte, a neuroblast, a fibroblast, a glioblast, a germ cell, hepatocyte, a renal cell, a sertoli cell, a chondrocyte, an epithelial cell, a cardiovascular cell, a keratinocyte, a smooth muscle cell, a cardiomyocyte, glial cell, a endothelial cell, a hormone-secreting cell, an immune cell, a pancreatic islet cell, a retina cell, and a neuron.

As used herein, “a first target comprising a first protein” does not mean that a first target consists of only a single protein. On the contrary, the first target can comprise more than one protein. It is not necessary that all the proteins are identical. Similarly, “a second target comprising a second protein” does not exclude that the second target comprises more than one protein. Moreover, all the proteins are not necessarily identical.

The first target is suitable to bind to a surface of a cell, preferably a genetically unmodified cell. The binding to the cell surface preferably comprises binding to a lipid or protein or combinations thereof, preferably a receptor or part thereof, that is expressed on the cell that is preferably a genetically unmodified cell. As used herein, the term “binding to a lipid or protein” encompasses a scenario where the first target binds to a (or several) glycolipid(s) or glycoprotein(s). In particular, the term encompasses the scenario that the first target only binds to the sugar moiety (or moieties) of said glycolipid or glycoprotein. The first target's binding to the lipid or protein or combinations thereof, preferably the receptor or part thereof, does not trigger biological effects. Examples of suitable receptors are selected from the group consisting of B cell receptor, T cell receptor, TNFalpha, ErbB1 (EGFR), ErbB2 (HER2) and ErbB4 (HER4), CD4, CD8, CD3, CD19, CD20, EpCAM, LDL receptor, PDGF receptor, FGF receptor, HGF receptor, NGF receptor, glutamate receptor, insulin receptor, and NMDA receptor.

In a preferred embodiment, the second target is essentially not suitable to adsorb or bind to a cell, preferably a genetically unmodified cell. Essentially no adsorption or binding to a cell translates into the fact that the second target itself has reduced biological activity as compared with the heterodimer. Reduced biological activity means a reduction of at least 50%, preferably at least 70%, more preferably at least 90%, most preferably at least 95%.

In a preferred embodiment, the second target comprises at least one molecule that is suitable to lead to a change in the molecular process of gene expression, DNA synthesis, gene silencing, RNA synthesis, preferably gene transcription, protein synthesis, peptide synthesis, post-translational modifications, preferably glycosylation, phosphorylation, ubiquitinylation, sumoylation, methylation or acetylation; cell growth; cell division; cell proliferation; cell survival; cell differentiation; cell ageing; organelle growth; organelle proliferation; polymerization or depolymerization of the cytoskeleton; viral replication; reverse transcription; phosphorylation of nucleosides; phosphorylation of nucleotides; apoptosis, necrosis, or antigen presentation.

In a particularly preferred embodiment, the second target comprises a liposome; an exosome; a DNA; preferably a viral DNA and/or a suicide gene; an RNA, preferably a viral RNA; a protein, preferably a reverse transcriptase, a viral thymidine kinase, a viral cytidine kinase, a viral adenine kinase, a viral guanine kinase, a virulence factor, a viral integrase, a protease, an apoptotic factor, a hormone having a nuclear receptor, preferably a steroid hormone, thyroid hormone, vitamin D, retinoic acid, NGF1-b; SF1-like protein, or GCNF-like protein; a transcription factor; or a small molecule, preferably an active ingredient. It is further preferred that the second target consists of a liposome; an exosome; a virus, mycobacterium, bacterium, protist or derivative thereof.

Preferably, the second target comprises a viral protein or derivatives thereof, preferably retroviral, lentiviral, adenoviral, adeno-associated viral, vesicular stomatitis viral, Newcastle Disease viral, herpes simplex viral, measles viral, pox viral, alphaviral, flaviral, rhabdoviral, picornaviruses or baculoviral protein or derivatives thereof, more preferably an AAV-2 protein or derivatives thereof. In this context, the second target may comprise a fusion protein comprising the second protein, wherein the fusion protein preferably comprises the full sequence or a partial sequence of a viral capsid protein, including VP1, VP2 and VP3 proteins. Preferably, the VP1, VP2 and VP3 proteins are of an AAV. Preferably, the second target comprising a viral protein or derivatives thereof bears mutations that essentially prevent the second target from adsorbing or binding to a cell. In an embodiment, the VP1, VP2 and VP3 proteins bear the mutations R585A and R588A.

Alternatively, however, the second protein is not fused to a viral capsid protein. This also applies to a scenario where the second target is viral or a derivative of a virus because some viruses do not have any capsid proteins. Thus, the second protein can also be fused to other proteins, e.g. membrane proteins, to be on the surface of enveloped viruses.

The second target may also comprise a viral protein derived from
Polyomaviridae
Adenoviridae
Herpesviridae
Poxviridae
Hepadnaviridae
Circoviridae
Parvoviridae
Reoviridae
Coronaviridae
Flaviviridae
Picornaviridae
Togaviridae
Retroviridae
Orthomyxoviridae
Paramyxoviridae
Rhabdoviridae.

A protein derived from one of said virus families shares at least 70% sequence homology with the respective wild-type viral protein, preferably at least 80% sequence homology, more preferably at least 85% sequence homology, even more preferably at least 90% sequence homology, most preferably at least 95% sequence homology such as 98 or 99% sequence homology. The second target may indeed consist of a virus as specified hereinbefore or a derivative thereof.

In an alternative embodiment, the second target may be a liposome; an exosome a mycobacterium, a bacterium, a protiste or derivative thereof.

For use in the present invention, any first and second proteins which can reversibly form a heterodimer upon irradiation with UV, visible or infrared light or in the dark can be used. In an embodiment, heterodimer forming proteins suitable for the present invention are those that upon irradiation with UV, visible or infrared light in a first wavelength range form a heterodimer, wherein the heterodimerization is reversed by irradiating the heterodimer with visible or infrared light in a second wavelength range. In an embodiment, a first protein suitable for the present invention has an absorbance maximum of the ground state within a narrow wavelength range which leads to a rapid conformational change to form an activated state. The first protein in the activated state is then able to form a heterodimer with the second protein. The activated state itself has an absorbance maximum (different from the absorbance maximum of the ground state) which converts the activated state of the first protein back to the ground state. Since the ground state of the first protein cannot form a heterodimer with the second protein, heterodimerization is reversed.

In the present invention, the first wavelength range and the second wavelength range are different. Two wavelength ranges are regarded as different not only if the wavelength ranges of the two wavelength ranges are different, but also if—given the same wavelength range—the intensities of the wavelengths within the wavelength ranges are different. For example, the first and second wavelength ranges may be 650 to 750 nm. The first wavelength range may have a peak wavelength (wavelength of highest intensity) at 660 nm, the second wavelength range may have a peak wavelength at 740 nm. According to the above definition, these first and second wavelength ranges are different.

In one embodiment, the first wavelength range includes the wavelength of the absorbance maximum of the ground state which converts the ground state of the first protein to its activated state, but it does not include the wavelength of the absorbance maximum of the activated state which converts the activated state of the first protein to its ground state. Preferably, the wavelength of the absorbance maximum of the ground state of the first protein has the highest intensity in the first wavelength range. Accordingly, the second wavelength range includes the wavelength of the absorbance maximum of the activated state which converts the activated state of the first protein to its ground state, but it does not include the wavelength of the absorbance maximum of the ground state which converts the ground state of the first protein to its activated state. Preferably, the wavelength of the absorbance maximum of the activated state of the first protein has the highest intensity in the second wavelength range.

In another embodiment, which is less preferred, the first wavelength range includes the wavelength of absorbance maximum of the ground state which converts the ground state of the protein to its activated state, and it also includes the wavelength of absorbance maximum of the activated state which converts the activated state of the protein to its ground state. However, the intensity of the wavelength of the absorbance maximum of the ground state is higher than the intensity of the wavelength of the absorbance maximum of the activated state. Accordingly, the second wavelength range includes the wavelength of absorbance maximum of the activated state which converts the activated state of the protein to its ground state, and it also includes the wavelength of absorbance maximum of the ground state which converts the ground state of the protein to its activated state. However, the intensity of the wavelength of the absorbance maximum of the activated state is higher than the intensity of the wavelength of the absorbance maximum of the ground state. Hence, in an embodiment of the invention, the first and second wavelength ranges can be identical from the point of view of the wavelengths, but are different from the point of view of the intensity of each wavelength. In an embodiment, the first wavelength range is in the visible light range (400 to less than 700 nm) or in the infrared range (700-1000 nm), in particular in the near-infrared range (700-790 nm). The second wavelength range is in the visible light range (400 to less than 700 nm) or in the infrared range (700-1000 nm), in particular in the near-infrared range (700-790 nm). In an embodiment of the present invention, if the first wavelength range is in the visible light range (400 to less than 700 nm), the second wavelength range is preferably in the infrared range (700-1000 nm), in particular in the near-infrared range (700-790 nm). Accordingly, if the first wavelength range is in the infrared range (700-1000 nm), in particular in the near-infrared range (700-790 nm), the second wavelength range is preferably in the visible light range (400 to less than 700 nm).

In a further embodiment, the first wavelength range is in the green part of the spectrum, and the second wavelength range is in the red part of the spectrum.

In a further embodiment, the first wavelength range is in the red part of the spectrum, and the second wavelength range is in the green part of the spectrum.

In a further embodiment, the first wavelength range is in the green part of the spectrum, and the second wavelength range is in the blue part of the spectrum.

In a further embodiment, the first wavelength range is in the blue part of the spectrum, and the second wavelength range is in the green part of the spectrum.

In a further embodiment, protein pairs of the invention also enter the binding or non-binding state in the dark. When exposed to light, they change to the non-binding or binding state accordingly.

First and second proteins to be used in the present invention are those which can heterodimerize fast, i.e. in less than 5 min. Heterodimerization can be easily detected using known methods, such as analytical ultracentrifugation or size-exclusion chromatography (SEC).

In an embodiment of the invention, the first protein is a phytochrome and the second protein is a phytochrome interacting partner. In a further embodiment, the first protein and the second protein may be represented by UV receptors, Cyanobacteriochromes, BLUF domains, LOV domains, Cryptochromes, or Fluorescent proteins. Exemplary first and second proteins are (conditions for interaction, i.e. heterodimer formation, and reversion, i.e. loss of heterodimers, are given in the last sentence of each bullet point):
UV receptors such as UVR8 (https://www.uniprotorg/uniprot/Q9FN03)/COP1 (https://www.uniprot.org/uniprot/P43254). Interaction: 300 nm, Reversion: dark.
Cyanobacteriochromes such as Amt c0023g2/BAm green (Interaction: 525 nm, Reversion: 680 nm), Amt c0023g2/BAm red. Interaction: 680 nm, Reversion: 525 nm (Front Microbiol. 2016 Apr. 26; 7:588 and https://doi.org/10.1101/769422).
BLUF domains such as PixD (https://www.uniprot.ora/uniprot/P74295)/PixE (https://www.uniprot.org/uniprot/P74294). Interaction: 450 nm, Reversion: dark.
LOV domains such as AsLOV2—SsrA/SspB (Proc Natl Acad Sci USA. 2015 Jan. 6; 112(1):112-7), AsLOV2/Zdk (Nat Methods. 2016 September; 13(9):755-8.), AsLOV2-ePDZpeptide/ePDZ (Nat Methods. 2012 Mar. 4; 9(4):379-84.), FKF1 (https://www.uniprot.org/uniprot/Q9C9W9)/GI (https://www.uniprot.org/uniprot/Q9SQI2), pMag/nMag (Nat Commun. 2015 Feb. 24; 6:6256.). Interaction: 450 nm, Reversion: dark.
Cryptochromes such as CRY2 (https://www.uniprot.org/uniprot/Q96524)/CIB1 (https://www.uniprot.org/uniprot/Q8GY61). Interaction: 450 nm, Reversion: dark.
Fluorescent proteins such as Dronpa145K/Dronpa145N (Science. 2012 Nov. 9; 338(6108):810-4.). Interaction: 400 nm, Reversion: 500 nm.

In the following, the kit comprising a phytochrome as first protein and a phytochrome interacting partner as second protein will be discussed in more detail. It is however noted that similar explanations apply, accordingly, for the systems described above.

In a preferred embodiment of the invention, the first protein is a phytochrome and the second protein is a phytochrome interacting partner. The phytochrome and the phytochrome interacting partner are not limited to the wild type, but also include a derivative thereof. A derivative of the phytochrome and phytochrome interacting partner contains mutations or deletions. “Deletion” as used herein means that at least one amino acid of the natural phytochrome is deleted. In one embodiment, the deletion may comprise 0.01% to 98% of the natural phytochrome or phytochrome interacting partner amino acid sequence. For example, a deletion of a phytochrome may be represented by the use of the photosensory module. Mutation as used herein means that at least one amino acid of the wild-type phytochrome is replaced by at least another amino acid. In one embodiment, the mutation may regard up to 3% of the natural phytochrome amino acid sequence.

Phytochromes are a class of photoreceptors in plants, bacteria and fungi used to detect light. They are sensitive to light in the red and far-red region of the visible spectrum and can be classed as either Type I, which are activated by far-red light, or Type II that are activated by red light. Phytochromes are characterized by a red/far-red photochromicity. Photochromic pigments change their “colour” (spectral absorbance properties) upon light absorption. In the case of phytochrome the ground state is Pr, the r indicating that it absorbs red light particularly strongly. The absorbance maximum is in most phytochromes a sharp peak 650-670 nm, so concentrated phytochrome solutions look turquoise-blue to the human eye. But once a red photon has been absorbed, the pigment undergoes a rapid conformational change to form the Pfr state. Here fr indicates that now not red but far-red (also called “near infra-red”; 700-790 nm) is preferentially absorbed. This shift in absorbance is apparent to the human eye as a slightly more greenish colour. When Pfr absorbs far-red light it is converted back to Pr. Hence, red light makes Pfr, far-red light makes Pr. In plants, at least Pfr is the physiologically active or “signalling” state.

Chemically, phytochrome consists of a chromophore, a single bilin molecule consisting of an open chain of four pyrrole rings, covalently bonded to the protein moiety via a highly conserved cysteine amino acid. It is the chromophore that absorbs light, and as a result changes the conformation of bilin and subsequently that of the attached protein, changing it from one state or isoform to the other. The phytochrome chromophore can be phytochromobilin, biliverdin or phycocyanobilin. Bilins are derived from the closed tetrapyrrole ring of haem by an oxidative reaction catalyzed by haem oxygenase to yield their characteristic open chain. Phytochrome interaction partners are proteins that can form heterodimers with the phytochromes. Generally, phytochrome interaction partners can form a heterodimer only with either the Pfr or the Pr form of the phytochrome.

In a further embodiment of the invention, the phytochrome is selected from the group comprising PhyA, PhyB, PhyC, PhyD, PhyE, BphP1 and the phytochrome interacting partner is selected from the group comprising PIF1, PIF2, PIF3, PIF4, PIF5, PIF6, PIF7, PIF8, FHY1, FHL, PpsR2, Q-PAS1.

In a further embodiment of the invention, the phytochrome is selected from the group consisting of PhyA, PhyB, PhyC, PhyD, PhyE, BphP1 and the phytochrome interacting partner is selected from the group consisting of PIF1, PIF2, PIF3, PIF4, PIF5, PIF6, PIF7, PIF8, FHY1, FHL, PpsR2, Q-PAS1.

The phytochrome may also be DrBphP and the phytochrome interacting partner an engineered antibody (or fragments thereof). Fragments include various derivatives of antibodies or biosimilars such as scFV, nanobodies and other antibody-like proteins. Antibody fragments, nanobodies and other antibody-like proteins binding to phytochrome in a light-dependent manner can be identified by a screening method as disclosed in Huang et al., 2020 (ACS Synth Biol. 2020 Nov. 12. doi: 10.1021/acssynbio.0c00397). This method is incorporated herein by reference.

In a preferred embodiment of the invention, the phytochrome is selected from the group consisting of PhyA, PhyB and BphP1 and the phytochrome interacting partner is selected from the group consisting of PIF3, PIF6, FHY1, FHL, PpsR2 and Q-PAS1.

As used herewith, PhyA, PhyB, PhyC, PhyD, PhyE, BphP1, PIF1, PIF2, PIF3, PIF4, PIF5, PIF6, PIF7, PIF8, FHY1, FHL, PpsR2 and Q-PAS1 are not limited to the wild type, but also include a derivative thereof. A derivative of the listed phytochromes and phytochrome interacting partners contains mutations or deletions. “Deletion” and “mutation” have been defined above.

PhyA, PhyB, PhyC, PhyD, PhyE are regulatory photoreceptors which exist in two forms that are reversibly interconvertible by light: the Pr form that absorbs maximally in the red region of the spectrum and the Pfr form that absorbs maximally in the far-red region. Photoconversion of Pr to Pfr induces an array of morphogenetic responses, whereas reconversion of Pfr to Pr cancels the induction of those responses. Preferably, PhyA, PhyB, PhyC, PhyD and PhyE are obtained from Arabidopsis thaliana. The sequence listings of these proteins can be found in https://www.uniprot.org/uniprot/P14712 (PhyA), https://www.uniprot.org/uniprot/P14713 (PhyB), https://www.uniprot.org/uniprot/P14714 (PhyC), https://www.uniprot.org/uniprot/P42497 (PhyD), https://www.uniprot.org/uniprot/P42498 (PhyE).

BphP1 is a bacterial phytochrome and is preferably obtained from Rhodopseudomonas palustris. The sequence listing of this protein can be found in https://www.uniprot.org/uniprot/A0A16115N6.

PIF1, PIF2, PIF3, PIF4, PIF5, PIF6, PIF7 and PIF8 are phytochrome interacting factors and are preferably obtained from Arabidopsis thaliana. The sequence listings of these proteins can be found in https://www.uniprot.org/uniprot/Q8GZM7 (PIF1), https://www.uniprot.org/uniprot/Q8L5W8-1 (PIF2), https://www.uniprot.org/uniprot/080536 (PIF3), https://www.uniprot.org/uniprot/Q8W2F3 (PIF4), https://www.uniprot.org/uniprot/Q84LH8 (PIF5), https://www.uniprot.org/uniprot/Q8L5W7 (PIF6), https://www.uniprot.org/uniprot/Q570R7 (PIF7), https://www.uniprot.org/uniprot/Q8GZ38-1 (PIF8).

FHY1 (far red elongated hypocotyl 1) and FHL are preferably obtained from Arabidopsis thaliana. The sequence listings of these proteins can be found in https://www.uniprot.org/uniprot/Q8 S4Q6 (FHY1) and https://www.uniprot.org/uniprot/A8MR65-1 (FHL). FHY1 and FHL, the only close FHY1 homolog in Arabidopsis, are small (23 and 20 kDa) plant-specific proteins which contain both functional NLS (nuclear localization signal) and NES (nuclear export signal) sequences. The homology between FHY1 and FHL is restricted to the NLS/NES region in their N-terminal half and to the extreme C-terminus. Although the overall similarity at the amino acid level is quite low (<30% identical amino acids), there is good evidence that FHY1 and FHL are nevertheless functional homologs (Plant Cell Physiol. 47(8): 1023-1034 (2006)).

PpsR2 is a transcriptional repressor and is preferably obtained from Rhodopseudomonas palustris (Nature Methods 2016 July; 13(7): 591-597). The sequence listing of this protein can be found in https://www.uniprot.org/uniprot/A0A167KP37. Q-PAS1 is a single-domain 17-kDa protein, which is derived from PpsR2 and lacks domains involved in the PpsR2 oligomerization (Nat Chem Biol. 2017 June; 13(6): 633-639). The sequence listing of this protein can be found in https://www.uniprot.org/uniprot/A0A167KP37.

PhyA, PhyB, PhyC, PhyD, PhyE can form heterodimers with PIF1, PIF2, PIF3, PIF4, PIF5, PIF6, PIF7 and PIF8. Both PhyA and PhyB form heterodimers with PIF3, PIF6, FHY1, FHL, leading to heterodimers PhyA/PIF3, PhyA/PIF6, PhyA/FHY1, PhyA/FHL, PhyB/PIF3, PhyB/PIF6, PhyB/FHY1, PhyB/FHL.

BphP1 forms heterodimers with both PpsR2 and Q-PAS1, leading to heterodimers BphP1/PpsR2 and BphP1/Q-PAS1.

In a preferred embodiment, the first protein and the second protein form heterodimers PhyA/PIF3, PhyA/PIF6, PhyA/FHY1, PhyA/FHL, PhyB/PIF3, PhyB/PIF6, PhyB/FHY1, PhyB/FHL, BphP1/PpsR2 and BphP1/Q-PAS1. More preferably, the heterodimers are PhyA/FHY1, PhyA/FHL, PhyB/PIF3, PhyB/PIF6, BphP1/PpsR2 and BphP1/Q-PAS1. Most preferably, the heterodimers are PhyB/PIF3 and PhyB/PIF6.

Heterodimerization occurs by irradiating the first protein and the second protein with visible or infrared light in a first specific wavelength range. Heterodimerization is generally fast (i.e., less than 5 min, but often in the range of seconds) and can be detected using known methods, such as analytical ultracentrifugation or size-exclusion chromatography (SEC). The heterodimerization can then be reversed by irradiating the heterodimer with visible or infrared light in a second specific wavelength range. The first specific wavelength range and the second specific wavelength range are different.

When the first protein is a phytochrome and the second protein is a phytochrome interacting partner, heterodimerization occurs by irradiating the phytochrome and the phytochrome interacting partner with visible or infrared light in a first specific wavelength range, wherein this range preferably includes the wavelength of maximum absorbance of the phytochrome in the ground state. The heterodimerization can then be reversed by irradiating the heterodimer with visible or infrared light in a second specific wavelength range, wherein this range preferably includes the wavelength of maximum absorbance of the phytochrome (activated state) in the heterodimer.

Specifically, when the phytochrome in the ground state is irradiated with a wavelength corresponding to the wavelength of maximum absorbance of the phytochrome in the ground state, the highest activation of the phytochrome, is observed.

Accordingly, when the phytochrome in the activated state (such as in the heterodimer) is irradiated with a wavelength corresponding to the wavelength of maximum absorbance of the phytochrome in the activated state, the highest concentration of phytochrome in the ground state is obtained. This corresponds to about 100% of the phytochrome.

When the phytochrome in the ground state is irradiated with light having a wavelength between the wavelength of maximum absorbance of the phytochrome in the ground state and the wavelength of maximum absorbance of the phytochrome in the activated state, the concentration of phytochrome in the activated state will be reduced compared to irradiation of the phytochrome in the ground state with light having the wavelength of maximum absorbance of the phytochrome in the ground state. The degree of reduction of activation increases with increasing distance from the wavelength of maximum absorbance of the phytochrome in the ground state. The same applies for the phytochrome in the activated state. When the phytochrome in the activated state is irradiated with light having a wavelength between the wavelength of maximum absorbance of the phytochrome in the ground state and the wavelength of maximum absorbance of the phytochrome in the activated state, the concentration of phytochrome in the ground state will be reduced compared to irradiation of the phytochrome in the activated state with light having the wavelength of maximum absorbance of the phytochrome in the activated state. The degree of reduction of inactivation increases with increasing distance from the wavelength of maximum absorbance of the phytochrome in the activated state. Hence, between the wavelength of maximum absorbance of the phytochrome in the ground state and the wavelength of maximum absorbance of the phytochrome in the activated state, a mixture of phytochrome in the ground state and in the activated state in varying ratios can be obtained.

When the phytochrome is selected from PhyA to PhyE, the first wavelength range preferably has the wavelength of 660 nm as peak wavelength.

When the phytochrome is selected from PhyA to PhyE, heterodimerization occurs by irradiating the kit of parts with light preferably in a wavelength range of 400 to less than 700 nm, more preferably 600-690 nm, even more preferably from 640-680 nm, most preferably 650-670 nm.

When the phytochrome is selected from PhyA to PhyE, the second wavelength range preferably has the wavelength of 740 nm as peak wavelength.

Reversion of the heterodimerization occurs in case of PhyA to PhyE by irradiating the heterodimer with light preferably in a wavelength range of 700-790 nm, more preferably 720-770 nm, even more preferably from 730-750 nm.

When the phytochrome is BphP1, the first wavelength range preferably has the wavelength of 760 nm as peak wavelength.

When the phytochrome is BphP1, heterodimerization occurs by irradiating the kit of parts with light preferably in a wavelength range of 700-900 nm, more preferably 720-780 nm, even more preferably from 750-770 nm.

When the phytochrome is BphP1, the second wavelength range preferably has the wavelength of 640 nm as peak wavelength.

Reversion of the heterodimerization occurs in case of BphP1 by irradiating the heterodimer with light preferably in a wavelength range of 500-690 nm, more preferably 620-670 nm, even more preferably from 630-650 nm.

Accordingly, PhyA/PIF3, PhyA/PIF6, PhyA/FHY1, PhyA/FHL, PhyB/PIF3, PhyB/PIF6, PhyB/FHY1 and PhyB/FHL heterodimers are formed by irradiating the corresponding monomers with light preferably in a wavelength range having the wavelength of 660 nm as peak wavelength. Preferably, the wavelength range is of 400 to less than 700 nm, more preferably 600-690 nm, even more preferably from 640-680 nm, most preferably 650-670 nm. Heterodimerization is reversed by irradiating the heterodimers with light preferably in a wavelength range having the wavelength of 740 nm as peak wavelength. Preferably, the wavelength range is of 700-790 nm, more preferably 720-770 nm, even more preferably from 730-750 nm.

Further, BphP1/PpsR2 and BphP1/Q-PAS1 heterodimers are formed by irradiating the corresponding monomers with light preferably in a wavelength range having the wavelength of 760 nm as peak wavelength. Preferably, the wavelength range is of 700-900 nm, more preferably 720-780 nm, even more preferably from 750-770 nm. Heterodimerization is reversed by irradiating the heterodimers with light preferably in a wavelength range having the wavelength of 640 nm as peak wavelength. Preferably, the wavelength range is of 500 to less than 700 nm, more preferably 620-670 nm, even more preferably from 630-650 nm.

Taken together, in a preferred embodiment, the first protein is a phytochrome and the second protein is a phytochrome interacting partner or vice versa. In this context, the phytochrome may be selected from the group comprising PhyA, PhyB, PhyC, PhyD, PhyE and BphP1 and the phytochrome interacting partner is selected from the group comprising PIF1, PIF2, PIF3, PIF4, PIF5, PIF6, PIF7, PIF8, FHY1, FHL, PpsR2 and Q-PAS1.

Coupling

In one embodiment of the present invention, the first protein and the second protein are coupled to the first target and the second target, respectively, via at least one covalent interaction or at least one non-covalent interaction. Accordingly, if the first target and the second target comprise more than one first protein and more than one second protein, respectively, each first protein and second protein is coupled to the first target and the second target, respectively, via at least one covalent interaction or at least one non-covalent interaction. Details of the coupling are disclosed in EP 20 178 474.1.

Preferably, the first protein and the second protein are coupled to the first target and the second target, respectively, via at least one covalent interaction. More preferably, the first protein and the second protein are coupled to the first target and the second target, respectively, via one single covalent interaction. When the first protein and the second protein are coupled to the first target and the second target, respectively, via one single covalent interaction, the risk is reduced that during the coupling reaction a protein molecule is coupled to two different targets, thus forming a covalent cross-link which is irreversible.

The type of covalent interaction is not limited, as long as it enables to couple the first protein and the second protein to the first target and the second target, respectively. In one embodiment of the invention, a covalent interaction is the result of the reaction between the first protein comprising a first chemical functional group and another element of the first target comprising a second chemical functional group which is able to chemically bond with the first chemical functional group. Accordingly, a further covalent interaction is the result of the reaction between the second protein comprising a third chemical functional group and another element of the second target comprising a fourth chemical functional group which is able to chemically bond with the third chemical functional group. In a preferred embodiment, the first and third functional groups of the first and second protein are the same. In a further preferred embodiment, also the second and fourth functional groups of the first and second targets are the same.

In a less preferred embodiment, the first or third chemical functional group are introduced to the first or second protein by means of a material having a first or third chemical functional group. Here, the first or third chemical functional group may be formed at a branch or end of the backbone of the first or second protein. Alternatively, the first or third chemical functional group may be formed within a linear first or second target.

In one embodiment, the first or second protein can be coupled to the first or second target, respectively, via at least one non-covalent interaction using the Streptavidin/Biotin system. Streptavidin is a 52.8 kDa protein which has an extraordinarily high affinity for biotin (also known as vitamin B7). With a dissociation constant (K_{d}) on the order of ≈10⁻¹⁴ mol/L, the binding of biotin to streptavidin is one of the strongest non-covalent interactions known in nature. Hence, Streptavidin may be introduced into the first or second target and biotin may be introduced into the first or second protein (or the other way round), leading to at least one non-covalent interaction between first or second target and the first or second protein, respectively, as soon as streptavidin interacts with biotin.

In another embodiment, the first or second protein can be coupled to the first or second target, respectively, via at least one non-covalent interaction using the His-tag/Ni-NTA system. The His-tag Ni-NTA interaction is based on the selectivity and high affinity of Ni-NTA (nickelnitrilotriacetic acid) for proteins containing an affinity tag of six or more consecutive Histidine residues. NTA, which has four chelating sites for nickel ions, is able to bind nickel tightly. Hence, Ni-NTA may be introduced into the first or second target and a His-tag may be introduced into the first or second protein (or the other way round), leading to at least one non-covalent interaction between first or second target and the first or second protein, respectively, as soon as Ni-NTA interacts with the His-tag.

Depending on the structure of the first and second target, more than one first proteins and second proteins can be coupled to the first target and the second target, respectively.

In an embodiment of the invention, at least one of the first and second protein are fusion proteins.

In this context, the first target may comprise an ankyrin repeat protein or derivative thereof. The ankyrin repeat protein or derivative thereof may be a designed ankyrin repeat protein (DARPᵢₙ). Preferably, this designed ankyrin repeat proteins (DARPin) binds to a cell surface protein. The cell surface protein may be selected from the group consisting of B cell receptor, T cell receptor, TNFalpha, ErbB1 (EGFR), ErbB2 (HER2) and ErbB4 (HER4), CD4, CD8, CD3, CD19, CD20, EpCAM, LDL receptor, PDGF receptor, FGF receptor, HGF receptor, NGF receptor, glutamate receptor, insulin receptor, and NMDA receptor.

The binding to the cell surface protein may have an affinity of at least 1 μM, preferably at least 100 nM, more preferably at least 10 nM, even more preferably 1 nM, and most preferably at least 100 pM.

The designed ankyrin repeat proteins (DARPin) may be generated according to methods known in the art. For example, it may be selected by ribosome display or filamentous phage display. The filamentous phage display may be a phage display employing the post-translational Sec pathway or a SRP phage display that employs the cotranslational signal recognition particle (SRP) pathway for the translocation of proteins to the periplasm.

Further in the above context, the second target may comprise a fusion protein comprising the second protein, wherein the fusion protein preferably comprises the full sequence or a partial sequence of a viral capsid protein, including VP1, VP2 and VP3 proteins. Preferably, the second target comprising a viral protein or derivatives thereof bears mutations that essentially prevent the second target from adhering or binding to a cell surface. In an embodiment, the VP1, VP2 and VP3 proteins bear at least one of the mutations R585A and R588A, preferably both.

Thus, in a preferred embodiment, the first target comprises an ankyrin repeat protein or derivative thereof and the second target comprises a fusion protein comprising the second protein, wherein the fusion protein preferably comprises the viral capsid of AAV-2, including VP1, VP2 and VP3 proteins. In a more preferred embodiment, the derivative of the ankyrin repeat protein is a designed ankyrin repeat protein (DARPin) which binds to a cell surface protein such as the EGF receptor (EGFR), in which case it can be referred to as DARPin_{EGFR}, and the second target comprises a fusion protein comprising the second protein, wherein the fusion protein comprises the full sequence of VP1, VP2 and VP3 proteins being blind to heparan sulfate proteoglycan (HSPG), for example VP1, VP2 and VP3 may bear mutations that essentially prevent the second target from adhering or binding to a cell surface, the mutations being preferably R585A and R588A. It is even more preferred that the first target comprises PhyB (e.g. PhyB-DARPin_{EGFR}) and the second target comprises PIF3 or PIF6 (e.g. an engineered AAV-2 capsid displaying PIF3 or PIF6 on its surface, wherein the VP1, VP2 and VP3 proteins are blind to binding sites at the cell surface such as heparan sulfate proteoglycan (HSPG), for example, because they bear mutations that essentially prevent the second target from adhering or binding to a cell surface, the mutations being preferably the mutations R585A and R588A).

Kit of Three Parts

The kit of parts according to the present invention may comprise an additional element. Thus, the first target and/or second the second target may comprise a third target. The third target may comprise a binding affinity between at least 1 μM, preferably at least 100 nM, more preferably at least 10 nM, even more preferably 1 nM, and most preferably at least 100 pM, for a protein or lipid that is present in an additional membrane-bound compartment within the cell, such as a mitochondrion. In this context, the binding affinity of the third target may be attributable to a third protein that is coupled to the third target.

Alternatively, the third target may comprise a nuclear localization signal, a mitochondrial targeting signal, a chloroplast targeting signal, an ER targeting signal.

The additional membrane-bound compartment may be selected from a mitochondrion, a plastid, a lysosome, a nucleus, an endoplasmatic reticulum, a Golgi apparatus, a peroxisome, and a glyoxysome. Preferably, the plastid is selected from the group consisting of a primary chloroplast, a secondary chloroplast, a tertiary chloroplast, a chromoplast, an amyloplast, a gerontoplast, rhodoplast, muroplast, leucoplast, elaioplast, proteinoplast and a tannosome.

Use

The present invention further relates to the use of the kit of parts as described hereinbefore for importing the second target, preferably as part of the heterodimer, into a cell, preferably an animal cell. In this context, the second target preferably comprises at least one DNA or RNA molecule. For example, the second target may comprise a virus or a derivative thereof, preferably AAV-2 or a derivative thereof. Since, upon irradiation, the second protein (e.g. a phytochrome interacting partner) comprised in the second target is able to form a heterodimer with the first protein (e.g. a phytochrome) comprised in the first target (see original claims 1 and 10), the virus or derivative thereof is an optogenetic viral system e.g., OptoAAV. Alternatively, the second target may comprise a liposome, exosome, mycobacterium, bacterium, protist or derivative thereof. In this case, the second target is an optogenetic system comprising a liposome, exosome, mycobacterium, bacterium, protist or derivative thereof.

Depending on the cell type, the kit of parts can be used to transfect or transduce the cell with the at least one DNA or RNA molecule mentioned above. In this context, the cell is an animal cell. With other cells, the kit of parts can be used to transform the cell. In this context, the cell is a bacterial cell, a fungus, an algae or a plant cell. Thus, the presents invention relates to the use of the inventive kit of parts, wherein the bacterial cell, the fungus, the algae or the plant cell is transformed with the at least one DNA or RNA molecule.

Finally, the kit of parts can be used to transduce a cell. In this context, the second target comprises a virus or a derivative thereof, preferably selected from the viruses disclosed above. Thus, the present invention relates to the use of the inventive kit of parts, wherein the cell is transduced by the virus or a derivative thereof. In particular, the present invention relates to the said use, wherein the transduction is spatiotemporally resolved in a multi-cell environment such as an organism, a tissue, a callus or a cell monolayer. Spatiotemporal resolution can be achieved by using a photomask such that heterodimers only form in an area that is irradiated. Spatiotemporal resolution can also be achieved by spatial illumination with a laser and one-photon or two-photon activation of the photoreceptor. Alternatively, spatiotemporal resolution can be achieved by directing the virus or derivative thereof to a surface epitope (e.g. a receptor protein expressed on the cell surface) that is only present on a subset of cells in a multi-cell environment such as an organism, a tissue, a callus or a cell monolayer. In a preferred embodiment, a derivative of an ankyrin repeat protein is used, more preferably a designed ankyrin repeat protein (DARPin) which binds to a cell surface protein such as the EGF receptor (EGFR), in which case it can be referred to as DARPin_{EGFR}. In a further embodiment, the photomask and the cell-specific second target (e.g. comprising DARPin_{EGFR}) are used in combination. Spatiotemporal resolution as described hereinbefore is further exemplified in the sections entitled “Engineered cell” and “Engineered tissue”.

The above considerations apply to transfection and transformation, too.

Moreover, the present invention relates to the use of the kit of parts according to the invention for delivering a suicide gene to tumor tissue. The suicide gene may be delivered using a AAV vector. As an example, the suicide gene is herpes simplex virus-thymidine kinase (HSV-TK), preferably under control of a strong and ubiquitously active virus promoter, more preferably a virus promoter. In addition, the tumor tissue may be exposed to an antitumor drug or an antiviral drug such as ganciclovir (GCV). In this example, the HSV-TK converts ganciclovir (GCV) into cytotoxic compounds inducing cell killing in HSV-TK expressing and neighboring cells within the tumor tissue.

In another embodiment, the present invention relates to the use of the kit of parts according to the invention, wherein the second target is treated to increase the accessibility of the second protein. Specifically, the second target may be exposed to one of a heat shock, a pH shift, a hypotonic treatment, a hypertonic treatment, a treatment with a chaotropic agent and a limited proteolysis. If the second target is exposed to a limited heat shock, the heat shock may comprise exposure to heat between 55° C. and 70° C. for 2 to 20 minutes, preferably between 60° C. and 65° C. for 5 to 15 minutes, more preferably 62.5° C. for 10 minutes. The heat shock (or heat treatment) preferably does not cause a substantial loss of biological activity e.g., loss of infectivity if a second target is used that comprises a virus or derivative thereof (e.g. OptoAAV). No substantial loss relates to a less than 10-fold loss, preferably a less than 9-fold loss, more preferably a less than 8-fold loss, even more preferably a less than 8-fold loss, further preferably a less than 8-fold loss, most preferably a less than 7-fold loss, and ideally a less than 6-fold loss. Thus, most preferably, a loss of infectivity is less than 7-fold, ideally less than 6-fold.

The present invention further relates to the use of the kit of parts as described hereinbefore for gene therapy in an organism, preferably a multicellular organism, more preferably an animal, even more preferably a human.

For the above-described uses, it does not matter whether the cell(s) is (are) first incubated with the first target, or first incubated with the second target, or incubated with both targets simultaneously.

Method

In a general aspect, the present invention relates to a method for importing a heterodimer as described hereinbefore into a cell which is preferably genetically unmodified, comprising
(i) preparing a kit of parts as described hereinbefore;
(ii) bringing the kit of parts into contact with the cell,
irradiating the kit of parts with UV, visible or infrared light in a first wavelength range to obtain a heterodimer of the first protein and the second protein.

For the above-described method, it does not matter whether, in step (ii), the cell is first brought into contact with the first target, or first brought into contact with the second target, or brought into contact with both targets simultaneously.

In a preferred embodiment, the present invention also relates to a method for importing DNA, RNA, protein or small molecule into a cell which is preferably genetically unmodified, comprising
(i) preparing a kit of parts as described hereinbefore;
(ii) bringing the kit of parts into contact with the cell,
irradiating the kit of parts with UV, visible or infrared light in a first wavelength range to obtain a heterodimer of the first protein and the second protein.

Concerning DNA and RNA, the embodiments described in the above section (entitled “Use”) are encompassed in analogy.

Specifically, the cell may preferably but without limitation be one or more selected from the group consisting of a bacterial cell, a fungal cell, an algae, a plant cell, a protoplast, an animal cell, preferably a mammalian cells, in particular human-derived stem cells, muscle-derived stem cells, dental pulp stem cells, nasal concha-derived mesenchymal stromal cells, an osteoblast, a myoblast, a tenocyte, a neuroblast, a fibroblast, a glioblast, a germ cell, hepatocyte, a renal cell, a sertoli cell, a chondrocyte, an epithelial cell, a cardiovascular cell, a keratinocyte, a smooth muscle cell, a cardiomyocyte, glial cell, a endothelial cell, a hormone-secreting cell, an immune cell, a pancreatic islet cell, a retina cell and a neuron.

By irradiation, the first protein and the second protein of the kit of parts form a heterodimer, thus cross-linking the first target and the second target which comprise the first protein and the second protein, respectively. This light-triggered cross-linking leads to uptake into a cell which is preferably genetically unmodified. Preferably, in the present invention heterodimer formation is not triggered by UV light, and it thus has minimal impact on the cell.

The wavelength range of the light used to irradiate the kit of parts depends on the first protein. Preferably, the wavelength range is in the visible light range (400 to less than 700 nm) or in the infrared range (700-1000 nm), in particular in the near-infrared range (700-790 nm). In an embodiment of the invention, visible light can be red, green or blue light, as defined above.

When the first protein is a phytochrome and the second protein is a phytochrome interacting partner, heterodimerization occurs by irradiating the phytochrome and the phytochrome interacting partner with visible or infrared light in a first specific wavelength range, wherein this range preferably includes the wavelength of maximum absorbance of the phytochrome.

The heterodimerization can then be reversed by irradiating the heterodimer with visible or infrared light in a second specific wavelength range, wherein this range preferably includes the wavelength of maximum absorbance of the phytochrome in the heterodimer.

Specifically, when the phytochrome in the ground state is irradiated with a wavelength corresponding to the wavelength of maximum absorbance of the phytochrome in the ground state, the highest activation of the phytochrome, is observed.

Accordingly, when the heterodimer, i.e. the phytochrome in the activated state, is irradiated with a wavelength corresponding to the wavelength of maximum absorbance of the phytochrome in the activated state, the highest concentration of phytochrome in the ground state is obtained, specifically all phytochrome molecules are in the ground state.

When the phytochrome in the ground state is irradiated with light having a wavelength between the wavelength of maximum absorbance of the phytochrome in the ground state and the wavelength of maximum absorbance of the phytochrome in the activated state, the concentration of phytochrome in the activated state will be reduced compared to irradiation of the phytochrome in the ground state with light having the wavelength of maximum absorbance of the phytochrome in the ground state. The degree of reduction of activation increases with increasing distance from the wavelength of maximum absorbance of the phytochrome in the ground state. The same applies for the phytochrome in the activated state. When the phytochrome in the activated state (i.e. in the heterodimer) is irradiated with light having a wavelength between the wavelength of maximum absorbance of the phytochrome in the ground state and the wavelength of maximum absorbance of the phytochrome in the activated state, the concentration of phytochrome in the ground state will be reduced compared to irradiation of the phytochrome in the activated state with light having the wavelength of maximum absorbance of the phytochrome in the activated state. The degree of reduction of inactivation increases with increasing distance from the wavelength of maximum absorbance of the phytochrome in the activated state. Hence, between the wavelength of maximum absorbance of the phytochrome in the ground state and the wavelength of maximum absorbance of the phytochrome in the activated state, a mixture of phytochrome in the ground state and in the activated state in varying ratios is present.

In one embodiment, the first wavelength range includes the absorbance maximum of the ground state which converts the ground state of the first protein (e.g. phytochrome) to its activated state, but it does not include the wavelength of absorbance maximum of the activated state which converts the activated state of the first protein (e.g. phytochrome) to its ground state. Preferably, the wavelength of the absorbance maximum of the ground state of the first proteon (e.g. phytochrome) has the highest intensity in the first wavelength range. Accordingly, the second wavelength range includes the wavelength of absorbance maximum of the activated state which converts the activated state of the first protein (e.g. phytochrome) to its ground state, but it does not include the wavelength of absorbance maximum of the ground state which converts the ground state of the first protein (e.g. phytochrome) to its activated state. Preferably, the wavelength of the absorbance maximum of the activated state of the first protein (e.g. phytochrome) has the highest intensity in the second wavelength range.

In another embodiment, which is less preferred, the first wavelength range includes the wavelength of absorbance maximum of the ground state which converts the ground state of the first protein (e.g. phytochrome) to its activated state, and it also includes the wavelength of absorbance maximum of the activated state which converts the activated state of the first protein (e.g. phytochrome) to its ground state. However, in the first wavelength range the intensity of the wavelength of the absorbance maximum of the ground state is higher than the intensity of the wavelength of the absorbance maximum of the activated state. Accordingly, the second wavelength range includes the wavelength of absorbance maximum of the activated state which converts the activated state of the first protein (e.g. phytochrome) to its ground state, and it also includes the wavelength of absorbance maximum of the ground state which converts the ground state of the first protein (e.g. phytochrome) to its activated state. However, in the second wavelength range the intensity of the wavelength of the absorbance maximum of the activated state is higher than the intensity of the wavelength of the absorbance maximum of the ground state. Hence, in an embodiment of the invention, the first and second wavelength ranges can be identical from the point of view of the wavelengths, but are different from the point of view of the intensity of each wavelength.

In an embodiment, the wavelengths of the absorbance maximum of the ground state or the activated state do not need to be the peak wavelengths in the first or second wavelength range. In cases where the wavelengths of the absorbance maximum of the ground state or the activated state are not the peak wavelengths in the first or second wavelength range, higher light intensities may be needed. However, if the wavelengths of the absorbance maximum of the ground state or the activated state are the peak wavelengths in the first or second wavelength range, a minimum of light intensity is needed.

As mentioned above, in case the first protein is a phytochrome comprising PhyA, PhyB, PhyC, PhyD, PhyE, preferably comprising PhyA or PhyB, the first wavelength range preferably has the wavelength of 660 nm as peak wavelength. Preferably, heterodimerization occurs by irradiating the kit of parts with light in a first wavelength range of 400 to less than 700 nm, more preferably 600-690 nm, even more preferably from 640-680 nm, most preferably 650-670 nm.

When the phytochrome is BphP1, the first wavelength range preferably has the wavelength of 760 nm as peak wavelength.

In case the first protein is a phytochrome comprising BphP1, heterodimerization occurs by irradiating the kit of parts with light preferably in a wavelength range of 700-900 nm, more preferably 720-780 nm, even more preferably from 750-770 nm.

As mentioned above, the second protein is preferably a phytochrome interacting partner which is selected in an embodiment from the group consisting of PIF1, PIF2, PIF3, PIF4, PIF5, PIF6, PIF7, PIF8, FHY1, FHL, PpsR2, Q-PAS1. The second protein is preferably selected from the group consisting of PIF3, PIF6, FHY1, FHL, PpsR2 and Q-PAS1.

Both PhyA and PhyB form heterodimers with PIF3, PIF6, FHY1, FHL, leading to heterodimers PhyA/PIF3, PhyA/PIF6, PhyA/FHY1, PhyA/FHL, PhyB/PIF3, PhyB/PIF6, PhyB/FHY1, PhyB/FHL.

BphP1 forms heterodimers with both PpsR2 and Q-PAS1, leading to heterodimers BphP1/PpsR2 and BphP1/Q-PAS1.

In a preferred embodiment, the first protein and the second protein form heterodimers PhyA/PIF3, PhyA/PIF6, PhyA/FHY1, PhyA/FHL, PhyB/PIF3, PhyB/PIF6, PhyB/FHY1, PhyB/FHL, BphP1/PpsR2 and BphP1/Q-PAS1. More preferably, the heterodimers are PhyA/FHY1, PhyA/FHL, PhyB/PIF3, PhyB/PIF6, BphP1/PpsR2 and BphP1/Q-PAS1. Most preferably, the heterodimers are PhyB/PIF3 and PhyB/PIF6.

Accordingly, PhyA/PIF3, PhyA/PIF6, PhyA/FHY1, PhyA/FHL, PhyB/PIF3, PhyB/PIF6, PhyB/FHY1 and PhyB/FHL heterodimers are formed by irradiating the corresponding monomers with light preferably in a first wavelength range having the wavelength of 660 nm as peak wavelength. Preferably, the first wavelength range is of 400 to less than 700 nm, more preferably 600-690 nm, even more preferably from 640-680 nm, most preferably 650-670 nm.

Further, BphP1/PpsR2 and BphP1/Q-PAS1 heterodimers are formed by irradiating the corresponding monomers with light preferably in a first wavelength range having the wavelength of 760 nm as peak wavelength. Preferably, the wavelength range is of 700-900 nm, more preferably 720-780 nm, even more preferably from 750-770 nm.

In an embodiment of the invention, the first protein is a phytochrome and the second protein is a phytochrome interacting partner. In a further embodiment, the first protein and the second protein may be represented by UV receptors, Cyanobacteriochromes, BLUF domains, LOV domains, Cryptochromes or Fluorescent proteins. Exemplary first and second proteins are:
UV receptors such as UVR8 (https://www.uniprot.org/uniprot/Q9FN03)/COP1 (https://www.uniprot.org/uniprot/P43254) Interaction: 300 nm, Reversion: dark
Cyanobacteriochromes such as Am1 c0023g2/BAm green (Interaction: 525 nm, Reversion: 680 nm), Am1 c0023g2/BAm red (Interaction: 680 nm, Reversion: 525 nm) (Front Microbiol. 2016 Apr. 26; 7:588 and https://doi.org/10.1101/769422).
BLUF domains such as PixD (https://www.uniprot.org/uniprot/P74295)/PixE (https://www.uniprot.org/uniprot/P74294) Interaction: 450 nm, Reversion: dark
LOV domains such as AsLOV2—SsrA/SspB (Proc Natl Acad Sci USA. 2015 Jan. 6; 112(1):112-7), AsLOV2/Zdk (Nat Methods. 2016 September; 13(9):755-8.), AsLOV2—ePDZpeptide/ePDZ (Nat Methods. 2012 Mar. 4; 9(4):379-84.), FKF1 (https://www.uniprotorg/uniprot/Q9C9W9)/GI (https://www.uniprot.org/uniprot/Q9SQI2), pMag/nMag (Nat Commun. 2015 Feb. 24; 6:6256.) Interaction: 450 nm, Reversion: dark
Cryptochromes such as CRY2 (https://www.uniprot.org/uniprot/Q96524)/CIB1 (https://www.uniprot.org/uniprot/Q8GY61). Interaction: 450 nm, Reversion: dark
Fluorescent proteins such as Dronpa145K/Dronpa145N (Science. 2012 Nov. 9; 338(6108):810-4.). Interaction: 400 nm, Reversion: 500 nm

In an embodiment, irradiating the kit of parts is carried out with a suitable light source, such as a LED, that emits light preferably of a specific wavelength or in a narrow wavelength range (max. 80 nm). In an embodiment, irradiation is carried out using a confocal microscope equipped with a red (e.g. 660 nm) and/or far-red (e.g. 740 nm) laser.

In an embodiment, the distance from the light source to the kit of parts and/or cell is preferably 1 cm to 30 cm.

In an embodiment, the photon intensity of the light is preferably 1-1000 μmol m⁻² s⁻¹, more preferably 10 to 200 μmol m⁻² s⁻¹.

In a further embodiment, the kit of parts is irradiated with light in a first wavelength range for a time period of 1 μs to 10 min, preferably 1 ms to 1 min, more preferably 100 ms to 10 s. At the end of this irradiation period, photoswitching is preferably completed. Complete crosslinking within the kit of parts by heterodimerization can be delayed in the range of seconds. Completion of crosslinking can be detected by analysing biological activity.

In the method of the present invention, heterodimerization of the first and second protein is preferably dependent on the dose of light. This means that the higher the light intensity at a given first wavelength range, the quicker is the heterodimerization.

In an embodiment of the present invention, if a first wavelength was in the visible light range (400 to less than 700 nm), a second wavelength range can be used to stop heterodimer formation. The second wavelength range is preferably in the infrared range (700-1000 nm), more preferably in the near-infrared range (700-790 nm). Accordingly, if the first wavelength range was in the infrared range (700-1000 nm), preferably in the near-infrared range (700-790 nm), the second wavelength range is preferably in the visible light range (400 to less than 700 nm).

Reversion of the heterodimerization occurs in case of PhyA to PhyE by irradiating the heterodimer with light in a second wavelength range preferably having the wavelength of 740 nm as peak wavelength. Further, the second wavelength range is preferably of 700-790 nm, more preferably 720-770 nm, even more preferably from 730-750 nm.

When the phytochrome is BphP1, the second wavelength range preferably has the wavelength of 640 nm as peak wavelength.

Reversion of the heterodimerization occurs in case of BphP1 by irradiating the heterodimer with light preferably in a wavelength range of 500-690 nm, more preferably 620-670 nm, even more preferably from 630-650 nm.

Accordingly, PhyA/PIF3, PhyA/PIF6, PhyA/FHY1, PhyA/FHL, PhyB/PIF3, PhyB/PIF6, PhyB/FHY1 and PhyB/FHL heterodimers are reversed by irradiating the heterodimers with light preferably in a second wavelength range having the wavelength of 740 nm as peak wavelength. Preferably, the second wavelength range is of 700-790 nm, more preferably 720-770 nm, even more preferably from 730-750 nm.

Further, BphP1/PpsR2 and BphP1/Q-PAS1 heterodimers are reversed by irradiating the heterodimers with light preferably in a second wavelength range having the wavelength of 640 nm as peak wavelength. Preferably, the second wavelength range is of 500 to less than 700 nm, more preferably 620-670 nm, even more preferably from 630-650 nm.

In another embodiment, the present invention relates to a method, wherein the second target is treated to increase the accessibility of the second protein. Specifically, the second target may be exposed to one of a heat shock, a hypotonic treatment, a pH shift, a hypertonic treatment, a treatment with a chaotropic agent and a limited proteolysis. If the second target is exposed to a limited heat shock, the heat shock may comprise exposure to heat between 55° C. and 70° C. for 2 to 20 minutes, preferably between 60 and 65° C. for 5 to 15 minutes, more preferably 62.5° C. for 10 minutes.

In a preferred aspect, the present invention also relates to a multi-step method for importing different heterodimers as described hereinbefore into a cell which is preferably genetically unmodified, comprising
(i) preparing a kit of parts as described hereinbefore;
(ii) bringing the kit of parts into contact with the cell;
(iii) irradiating the kit of parts with UV, visible or infrared light in a first wavelength range to obtain a heterodimer of the first protein and the second protein;
(iv) optionally providing time and other conditions suitable to promote import into the cell;
(v) reversing heterodimer formation by irradiating the cell and/or heterodimer with UV, visible or infrared light in a second wavelength range or in the dark, wherein the second wavelength range is different from the first wavelength range,
(vi) repeating steps (i) to (iii) and optionally (iv) with a different kit of parts which may comprise a different first target, or a different second target or both a different first and second target.

In a further preferred embodiment, the present invention also relates to a multi-step method for importing DNA, RNA, protein or small molecule into a cell which is preferably genetically unmodified, comprising
(i) preparing a kit of parts as described hereinbefore;
(ii) bringing the kit of parts into contact with the cell;
(iii) irradiating the kit of parts with UV, visible or infrared light in a first wavelength range to obtain a heterodimer of the first protein and the second protein;
(iv) optionally providing time and other conditions suitable to promote import into the cell;
(v) reversing heterodimer formation by irradiating the cell and/or heterodimer with UV, visible or infrared light in a second wavelength range or in the dark, wherein the second wavelength range is different from the first wavelength range,
(vi) repeating steps (i) to (iii) and optionally (iv) with a different kit of parts which may comprise a different first target, or a different second target or both a different first and second target.

This preferred method is illustrated as an example in FIG. 5B. It shows the spatiotemporally resolved transduction with two different transgenes. A-431 cells were transduced spatially resolved, using a photomask 1 and OptoAAV_{GFP}. Following an incubation in the dark and washing with PBS, the same procedure was repeated using a photomask 2 and OptoAAVm_{Scarlet}. After the second 2 h incubation step in the dark, cells were washed with PBS and incubated for 44 h in cell culture medium under 740 nm illumination. Of course, this is just an illustrative example and the invention could also be carried out with other transgenes such as two different therapeutic transgenes.

It is noted that, in this example, first and second proteins are activated with light, and then return to the ground state upon irradiation with infrared light. Thus, heterodimers are obtained with light and lost with light. However, it is also within the scope of the present invention that incubation in the dark leads to loss (i.e. dissociation) of heterodimers. This takes however more time, and is thus less preferable than dissociation using light.

Spatiotemporal resolution, as illustrated in FIG. 5B, can be achieved by using a photomask such that heterodimers only form in an area that is irradiated. Alternatively, spatiotemporal resolution can be achieved by directing the kit of parts to a surface epitope (e.g. a receptor protein expressed on the cell surface) that is only present on a subset of cells in a multi-cell environment such as an organism, a tissue, a callus or a cell monolayer. In a preferred embodiment, a derivative of an ankyrin repeat protein is used, more preferably a designed ankyrin repeat protein (DARPin) which binds to a cell surface protein such as the EGF receptor (EGFR), in which case it can be referred to as DARPin_{EGFR}. In a further embodiment, the photomask and the cell-specific second target (e.g. comprising DARPin_{EGFR}) are used in combination. Spatiotemporal resolution as described hereinbefore is further exemplified in the sections entitled “Engineered cell” and “Engineered tissue”.

If the activation of the targets in the claimed kit of parts was not reversible, the second-step or second-round (see step vi) second target would also bind to first targets activated in the first step or round since they will likely not all be used up by second targets in the first step or round. Thus, the reversibility is advantageous.

Nucleotide Sequences

Moreover, the present invention relates to a kit of parts comprising
a first nucleotide sequence encoding a first target protein,
a second nucleotide sequence encoding a second target protein,
   wherein the first target protein and the second target protein are suitable for assembly into a first target and a second target as described hereinbefore, respectively,
   wherein the first target comprises the sequence of a first protein and the second target comprises the sequence of a second protein, and
   wherein the first protein and the second protein are suitable to form a heterodimer upon irradiation with visible or infrared light in a first wavelength range, which can be reversed upon irradiating the heterodimer with visible or infrared light in a second wavelength range or in the dark, wherein the second wavelength range is different from the first wavelength range.

The first and/or second nucleotide sequences are preferably DNA sequences. Moreover, the first and/or second target proteins may be fusion proteins comprising the sequences of the first and second proteins, respectively.

Consequently, the disclosure of the first and second proteins according to the invention can be directly and unambiguously translated into a teaching for a kit of parts comprising nucleotide sequences. If, for example, the first protein is phytochrome which is selected from the group comprising PhyA, PhyB, PhyC, PhyD, PhyE, BphP1 and the second protein is a phytochrome interacting partner which is selected from the group comprising PIF1, PIF2, PIF3, PIF4, PIF5, PIF6, PIF7, PIF8, FHY1, FHL, PpsR2, Q-PAS1, then the kit of parts comprising nucleotide sequences comprises the nucleotide sequences corresponding to these proteins. These nucleotide sequences are part of the first and second nucleotide sequences which preferably encode fusion proteins comprising the sequences of the first and second proteins.

In a preferred embodiment, the first target protein comprises or consists of a fusion protein. This fusion protein is encoded by the first nucleotide sequence and comprises, besides the sequence of the first protein (e.g. PhyA, PhyB, PhyC, PhyD, PhyE, BphP1), the sequence of an ankyrin repeat protein or derivative thereof. The ankyrin repeat protein or derivative thereof may be a designed ankyrin repeat protein (DARPin). Preferably, this designed ankyrin repeat proteins (DARPin) binds to a cell surface protein. The cell surface protein may be selected from the group consisting of B cell receptor, T cell receptor, TNFalpha, ErbB1 (EGFR), ErbB2 (HER2) and ErbB4 (HER4), CD4, CD8, CD3, CD19, CD20, EpCAM, LDL receptor, PDGF receptor, FGF receptor, HGF receptor, NGF receptor, glutamate receptor, insulin receptor, and NMDA receptor.

The designed ankyrin repeat proteins (DARPin) may have been generated according to methods known in the art as described above.

In a preferred embodiment, the second target protein comprises or consists of a second fusion protein comprising the sequence of a viral protein, preferably a viral capsid protein such as VP1, VP2 and/or VP3. Moreover, the second fusion protein preferably comprises the sequence of a second protein, wherein the second protein is preferably the active phytochrome binding (APB) domain of PIF1, PIF2, PIF3, PIF4, PIF5, PIF6, PIF7, or PIF8.

In addition, the second nucleotide sequence encoding the second fusion protein may be under the control of a promoter, preferably a CMV promoter, preferably on a plasmid, preferably a pMH303 plasmid.

The kit of parts may further comprise a helper plasmid, preferably an AAV helper plasmid. Preferably, the helper plasmid encodes the AAV-2 nonstructural proteins Rep78, Rep68, Rep52, Rep40, AAP as well as the capsid proteins VP1 and VP3. Moreover, the helper plasmid may be pRCVP2koA.

In one embodiment, the start codon of native VP2 is deleted in the helper plasmid which preferably is the plasmid pRCVP2koA. The helper plasmid, preferably the plasmid pRCVP2koA, may encode the mutations R585A and R588A in the cap genes.

Virus

Furthermore, the present invention relates a virus or a derivative thereof comprising
a second protein as described hereinbefore,
wherein the virus or a derivative thereof has essentially no biological activity in the absence of a first target as described hereinbefore,
wherein the second protein and the first protein are suitable to form a heterodimer upon irradiation with UV, visible or infrared light in a first wavelength range, which can be reversed upon irradiating the heterodimer with UV, visible or infrared light in a second wavelength range or in the dark, wherein the second wavelength range is different from the first wavelength range.

Concerning the virus or a derivative thereof and the second protein, all embodiments described hereinbefore are encompassed and apply in analogy.

Nucleotide Sequence (Plasmid)

The present invention also relates to a viral vector comprising
a protein X,
wherein the protein X is suitable to form a heterodimer with a first protein upon irradiation with visible or infrared light in a first wavelength range, which can be reversed upon irradiating the heterodimer with visible or infrared light in a second wavelength range or in the dark, wherein the second wavelength range is different from the first wavelength range.

The protein X is synonymous with the second protein according to the present invention. Thus, the disclosure of the first and second proteins according to the invention can be directly and unambiguously translated into a teaching for a viral vector as specified hereinabove.

A nucleotide sequence encoding protein X is preferably a DNA sequence which is preferably part of a plasmid (e.g. a pMH303 plasmid). In one embodiment, the DNA sequence encodes a fusion protein comprising the sequence of the second protein (such as the active phytochrome binding (APB) domain of PIF1, PIF2, PIF3, PIF4, PIF5, PIF6, PIF7, or PIF8) and a further protein. The further protein may be the viral capsid protein VP2.

The second protein may be the active phytochrome binding (APB) domain of PIF1, PIF2, PIF3, PIF4, PIF5, PIF6, PIF7, or PIF8.

The nucleotide sequence encoding the second fusion protein may be under the control of a promoter, preferably a CMV promoter, preferably on a plasmid, preferably a pMH303 plasmid.

Concerning the viral vector and protein X, all embodiments described hereinbefore are encompassed and apply in analogy.

Engineered Cell

The present invention also relates to a single cell or cells obtained by transduction, transfection, or transformation as disclosed herein.

Engineered Tissue

Furthermore, the present invention relates to an engineered tissue comprising engineered and non-engineered cells. This is because the kit of parts as described hereinbefore represents a tool for locally engineering individual cells or cell clusters by using locally restricted beams of low doses of cell friendly light, preferably not UV, while sparing the surrounding cells. Since the kit of parts can be de-activated quickly and under mild conditions, cells can be easily engineered in a spatio-temporal manner.

Adapter Protein

In another aspect, the present invention relates to an adapter protein, wherein the adapter protein and a second protein are suitable to form a heterodimer upon irradiation with UV, visible or infrared light in a first wavelength range or in the dark, which can be reversed upon irradiating the heterodimer with UV, visible or infrared light in a second wavelength range or in the dark, wherein the second wavelength range is different from the first wavelength range. The heterodimer is adapted to have biological activity upon exposure to at least one cell, preferably a genetically unmodified cell.

An exemplary adaptor protein may comprise an ankyrin repeat protein or derivative thereof. The ankyrin repeat protein or derivative thereof may be a designed ankyrin repeat protein (DARP1). Preferably, this designed ankyrin repeat proteins (DARPin) binds to a cell surface protein. The cell surface protein may be selected from the group consisting of B cell receptor, T cell receptor, TNFalpha, ErbB1 (EGFR), ErbB2 (HER2) and ErbB4 (HER4), CD4, CD8, CD3, CD19, CD20, EpCAM, LDL receptor, PDGF receptor, FGF receptor, HGF receptor, NGF receptor, glutamate receptor, insulin receptor, and NMDA receptor.

The binding to the cell surface protein may have an affinity of at least 1 μM, preferably at least 100 nM, more preferably at least 10 nM, even more preferably 1 nM, and most preferably at least 100 pM.

The designed ankyrin repeat proteins (DARPin) may be generated according to methods known in the art. For example, it may be selected by ribosome display or filamentous phage display. The filamentous phage display may be a phage display employing the post-translational Sec pathway or a SRP phage display that employs the co-translational signal recognition particle (SRP) pathway for the translocation of proteins to the periplasm.

In a more embodiment, the designed ankyrin repeat protein (DARPin) binds to a cell surface protein such as the EGF receptor (EGFR), in which case it can be referred to as DARPin_{EGFR}, more preferably, the designed ankyrin repeat protein (DARPin) comprises PhyB (PhyB-DARPin_{EGFR})

An exemplary adaptor protein may comprise PhyB which is suitable to interact with PIF6 upon irradiation with visible light with a wavelength of about 660 nm, PhyB is activated and the activated PhyB interacts with PIF6 to form a heterodimer. The heterodimer can then be separated by irradiating it with infrared light with a wavelength of about 740 nm. Hereby, the activated state of PhyB is reversed. The ground state of PhyB cannot interact with PIF6, so that heterodimerization is reversed, the contacts-links between the first and second targets are thus separated. Hence, the adaptor protein is re-obtained which can be used again by irradiating it with visible light with a wavelength of about 660 nm. Heterodimer formation is thus a reversible process in the present invention.

For use in the present invention, any adaptor protein which can reversibly form a heterodimer upon irradiation with UV, visible or infrared light or in the dark can be used. Details have been given above in the context of the first target.

The adaptor protein may be selected from the group comprising PhyA, PhyB, PhyC, PhyD, PhyE, BphP1.

In a further embodiment of the invention, the adaptor protein is selected from the group consisting of PhyA, PhyB, PhyC, PhyD, PhyE, BphP1.

In a preferred embodiment of the invention, the adaptor protein is selected from the group consisting of PhyA, PhyB and BphP1.

As used herewith, PhyA, PhyB, PhyC, PhyD, PhyE, BphP1, PIF1, PIF2, PIF3, PIF4, PIF5, PIF6, PIF7, PIF8, FHY1, FHL, PpsR2 and Q-PAS1 are not limited to the wild type, but also include a derivative thereof. A derivative of the listed phytochromes and phytochrome interacting partners contains mutations or deletions. “Deletion” and “mutation” have been defined above.

PhyA, PhyB, PhyC, PhyD, PhyE can form heterodimers with PIF1, PIF2, PIF3, PIF4, PIF5, PIF6, PIF7 and PIF8. Both PhyA and PhyB form heterodimers with PIF3, PIF6, FHY1, FHL, leading to heterodimers PhyA/PIF3, PhyA/PIF6, PhyA/FHY1, PhyA/FHL, PhyB/PIF3, PhyB/PIF6, PhyB/FHY1, PhyB/FHL.

BphP1 forms heterodimers with both PpsR2 and Q-PAS1, leading to heterodimers BphP1/PpsR2 and BphP1/Q-PAS1.

In a preferred embodiment, the adapter protein and the second protein form heterodimers PhyA/PIF3, PhyA/PIF6, PhyA/FHY1, PhyA/FHL, PhyB/PIF3, PhyB/PIF6, PhyB/FHY1, PhyB/FHL, BphP1/PpsR2 and BphP1/Q-PAS1. More preferably, the heterodimers are PhyA/FHY1, PhyA/FHL, PhyB/PIF3, PhyB/PIF6, BphP1/PpsR2 and BphP1/Q-PAS1. Most preferably, the heterodimers are PhyB/PIF3 and PhyB/PIF6.

Heterodimerization occurs by irradiating the adapter protein and the second protein with visible or infrared light in a first specific wavelength range. Heterodimerization is generally fast (i.e., less than 5 min, but often in the range of seconds) and can be detected using known methods, such as analytical ultracentrifugation or size-exclusion chromatography (SEC). The heterodimerization can then be reversed by irradiating the heterodimer with visible or infrared light in a second specific wavelength range. The first specific wavelength range and the second specific wavelength range are different.

When the adapter protein is a phytochrome and the second protein is a phytochrome interacting partner, heterodimerization occurs by irradiating the phytochrome and the phytochrome interacting partner with visible or infrared light in a first specific wavelength range, wherein this range preferably includes the wavelength of maximum absorbance of the phytochrome in the ground state. The heterodimerization can then be reversed by irradiating the heterodimer with visible or infrared light in a second specific wavelength range, wherein this range preferably includes the wavelength of maximum absorbance of the phytochrome (activated state) in the heterodimer.

Specifically, when the phytochrome in the ground state is irradiated with a wavelength corresponding to the wavelength of maximum absorbance of the phytochrome in the ground state, the highest activation of the phytochrome, is observed.

Accordingly, when the phytochrome in the activated state (such as in the heterodimer) is irradiated with a wavelength corresponding to the wavelength of maximum absorbance of the phytochrome in the activated state, the highest concentration of phytochrome in the ground state is obtained. This corresponds to about 100% of the phytochrome. Further details have been specified above.

Alternatively, the adapter protein and the second protein may comprise the following proteins:
UV receptors such as UVR8 (https://www.uniprot.org/uniprot/Q9FN03)/COP1 (https://www.uniprot.org/uniprot/P43254) Interaction: 300 nm, Reversion: dark
Cyanobacteriochromes such as Amt c0023g2/BAm green (Interaction: 525 nm, Reversion: 680 nm), Am1 c0023g2/BAm red (Interaction: 680 nm, Reversion: 525 nm) (Front Microbiol. 2016 Apr. 26; 7:588 and https://doi.org/10.1101/769422).
BLUF domains such as PixD (https://www.uniprot.org/uniprot/P74295)/PixE (https://www.uniprot.org/uniprot/P74294) Interaction: 450 nm, Reversion: dark
LOV domains such as AsLOV2-SsrA/SspB (Proc Natl Acad Sci USA. 2015 Jan. 6; 112(1):112-7), AsLOV2/Zdk (Nat Methods. 2016 September; 13(9):755-8.), AsLOV2-ePDZpeptide/ePDZ (Nat Methods. 2012 Mar. 4; 9(4):379-84.), FKF1 (https://www.uniprot.org/uniprot/Q9C9W9)/GI (https://www.uniprot.org/uniprot/Q9SQI2), pMag/nMag (Nat Commun. 2015 Feb. 24; 6:6256.) Interaction: 450 nm, Reversion: dark
Cryptochromes such as CRY2 (https://www.uniprot.org/uniprot/Q96524)/CIB1 (https://www.uniprot.org/uniprot/Q8GY61). Interaction: 450 nm, Reversion: dark
Fluorescent proteins such as Dronpa145K/Dronpa145N (Science. 2012 Nov. 9; 338(6108):810-4.). Interaction: 400 nm, Reversion: 500 nm

### Embodiments

1. A kit of parts having biological activity, the kit of parts comprising
   a first target comprising a first protein,
   a second target comprising a second protein,
wherein the first protein and the second protein are suitable to form a heterodimer upon irradiation with UV, visible or infrared light in a first wavelength range or in the dark, which can be optionally reversed upon irradiating the heterodimer with UV, visible or infrared light in a second wavelength range or in the dark, wherein the second wavelength range is different from the first wavelength range, wherein the biological activity consists of triggering both the uptake of DNA, RNA, proteins, or small molecules into a cell which is preferably genetically unmodified, and biological effects, and
characterized in that at least one of the first and the second target itself has reduced biological activity as compared with the heterodimer.
2. The kit of parts of embodiment 1, wherein the first target is suitable to bind to a lipid or protein or combinations thereof, preferably a receptor or part thereof, that is expressed on a cell, preferably a genetically unmodified cell.
3. The kit of parts of embodiment 2, wherein the first target's binding to the lipid or protein or combinations thereof, preferably the receptor or part thereof, does not trigger biological effects.
4. The kit of parts of any one of embodiments 1 to 3, wherein the second target is essentially not suitable to adsorb or bind to a cell, preferably a genetically unmodified cell.
5. The kit of parts of any one of embodiments 1 to 4, wherein the second target comprises at least one molecule that is suitable to lead to a change in the molecular process of gene expression, DNA synthesis, gene silencing, RNA synthesis, preferably gene transcription, protein synthesis, peptide synthesis, post-translational modifications, preferably glycosylation, phosphorylation, ubiquitinylation, sumoylation, methylation or acetylation; cell growth; autophagy; mitophagy; cell death; cell division; cell proliferation; cell survival; cell differentiation; cell ageing; organelle growth; organelle proliferation; polymerization or depolymerization of the cytoskeleton; viral replication; reverse transcription; phosphorylation of nucleosides; phosphorylation of nucleotides; apoptosis, necrosis, or antigen presentation.
6. The kit of parts of any one of embodiments 1 to 5, wherein the second target comprises a liposome, an exosome, a DNA; preferably a viral DNA and/or a suicide gene; an RNA, preferably a viral RNA; a protein, preferably a reverse transcriptase, a viral thymidine kinase, a viral cytidine kinase, a viral adenine kinase, a viral guanine kinase, a virulence factor, a viral integrase, a protease, an apoptotic factor, a hormone having a nuclear receptor, preferably a steroid hormone, thyroid hormone, vitamin D, retinoic acid, NGF1-b; SF1-like protein, or GCNF-like protein; a transcription factor; a small molecule, preferably an active ingredient.
7. The kit of parts of any one of embodiments 1 to 6, wherein the second target comprises a viral protein or derivatives thereof, preferably retroviral, lentiviral, adenoviral, adeno-associated viral, vesicular stomatitis viral, Newcastle Disease viral, herpes simplex viral, measles viral, pox viral, alphaviral, flaviral, rhabdoviral, picornaviruses or baculoviral protein or derivatives thereof, more preferably an AAV-2 protein or derivatives thereof.
8. The kit of parts of any one of embodiments 1 to 7, wherein the second target comprises a fusion protein comprising the second protein, wherein the fusion protein preferably comprises the full sequence or a partial sequence of a viral capsid protein, including VP1, VP2 and VP3 proteins of AAV.
9. The kit of parts of embodiment 8, wherein the VP1, VP2 and VP3 proteins bear the mutations R585A and R588A.
10. The kit of parts of any one of embodiments 1 to 9, wherein the first protein is a phytochrome and the second protein is a phytochrome interacting partner or vice versa.
11. The kit of parts of embodiment 10, wherein the phytochrome is selected from the group comprising PhyA, PhyB, PhyC, PhyD, PhyE, BphP1 and DrBphP and the phytochrome interacting partner is selected from the group comprising PIF1, PIF2, PIF3, PIF4, PIF5, PIF6, PIF7, PIF8, FHY1, FHL, PpsR2, Q-PAS1 and engineered antibodies (or fragments thereof).
12. Use of the kit of parts of any one of embodiments 1 to 11 for importing the second target into a membrane-bound compartment, preferably a cell, more preferably an animal cell.
13. Use according to embodiment 12, wherein the second target comprises a virus or a derivative thereof, preferably AAV-2 or a derivative thereof.
14. A method for importing DNA, RNA, protein or small molecule into a cell which is preferably genetically unmodified, comprising
   (i) preparing a kit of parts of any one of embodiments 1 to 11;
   (ii) bringing the kit of parts into contact with the cell,
   irradiating the kit of parts with visible or infrared light in a first wavelength range to obtain a heterodimer of the first protein and the second protein.
15. A kit of parts comprising
   a first nucleotide sequence encoding a first target protein,
   a second nucleotide sequence encoding a second target protein,
wherein the first target protein and the second target protein are suitable for assembly into a first target and a second target as defined in embodiment 1, respectively,
wherein the first target is coupled to a first protein through the first target protein and the second target is coupled to a second protein through the second target protein, and
wherein the first protein and the second protein are suitable to form a heterodimer upon irradiation with visible or infrared light in a first wavelength range, which can be optionally reversed upon irradiating the heterodimer with visible or infrared light in a second wavelength range, wherein the second wavelength range is different from the first wavelength range.

### Examples

It is referred to the Examples of the priority document EP 20 208 920.7 which are herewith included by reference. Those Examples were supplemented by other experiments, as explained below. The following Examples have been published in Homer et al., Sci. Adv. 2021; vol. 7; issue 25 (DOI: 10.1126/sciadv.abf0797).

Materials and Methods

Cloning of Plasmids

The nucleic acid sequences of all plasmids generated in this study are depicted in FIG. T2. The plasmids were assembled by Gibson or AQUA cloning and the used templates for PCR are mentioned in FIG. T2. The codon-optimized (for E. coli) sequences for the DARPins Ec1 (EpCAM), 9_29 (Her2/ErbB2) and D55.2 (CD4) and the codon-optimized (for human and E. coli) sequence for SpyCatcher003 were ordered as gBlocks from IDT (Coralville, IA). All other sequences or mutations were introduced with oligonucleotides and PCR.

Illumination

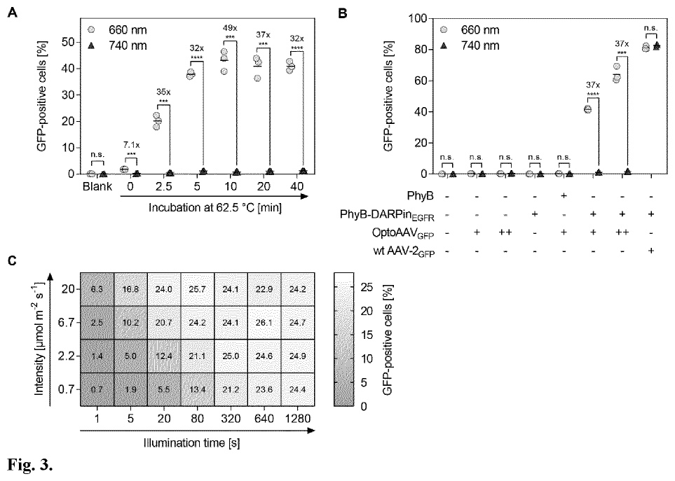

If not indicated otherwise, samples were illuminated with microcontroller-regulated illumination panels containing LEDs with ˜660 nm (LED660N-03, Roithner Lasertechnik, Vienna, Austria, peak wavelength: 660 nm; LH WSAM, Osram Opto Semiconductors, Regensburg, Germany, peak wavelength: 660 nm; LST1-01F06-PRD1-00, Opulent Americas, Raleigh, NC, peak wavelength: 655 nm) or 740 nm (LED740-01AU, SMB1N-740D, both Roithner Lasertechnik; LZ4-00R308, LED Engin, San Jose, CA) peak wavelengths. For spatially resolved illumination, custom laser photoplot films (4000 DPI, JD Photo Data, Herts, UK) were used as photomask. The experiment depicted in FIG. 3C was performed with the optoPlate-96 equipped with 630 nm (150141RB73100, Wurth Elektronik, Niedernhall, Germany) and 780 nm (SMT780-27, Marubeni, Tokio, Japan) LEDs and illumination protocols were defined with optoConfig-96. For illumination at the confocal microscope, a pE-4000 LED light source (CoolLED, Andover, UK) with 740 nm was used. After illumination of the samples with the indicated wavelengths, they were only handled under dim green safe light until the end of the experiment to prevent photoswitching of PhyB. Light intensities were measured with an AvaSpec-ULS2048 fiber-optic spectrometer (Avantes BV, Apeldoorn, Netherlands). Where indicated, pulsed 660 nm illumination (5 min ON, 55 min OFF) was used to prevent continuous cycling of PhyB between the binding and non-binding state as occurring under continuous 660 nm light. If not indicated otherwise, samples were illuminated at an intensity of 20 μmol m⁻² s⁻¹.

Protein Production and Purification

For production of the different PhyB-DARPin proteins and PhyB-mCherry-SpyTag, the corresponding plasmid (see FIG. T2) encoding the fusion protein as well as the biosynthesis enzymes for the chromophore phycocyanobilin (PCB) was transformed into E. coli BL21 Star (DE3) (Thermo Fisher Scientific, Waltham, MA, cat. no. C601003) and transformed bacteria were selected in LB medium supplemented with 100 μg ml⁻¹ streptomycin. Bacteria were grown at 30° C. and 150 rpm to an OD₆₀₀ of 0.8 and expression was induced by the addition of 1 mM IPTG. After incubation for 20 h at 18° C. in the dark, bacteria were harvested by centrifugation at 6,500 g, resuspended in lysis buffer (50 mM NaH₂PO₄, 300 mM NaCl, 10 mM imidazole, 0.5 mM tris(2-carboxyethyl)phosphine (TCEP), pH 8.0), shock frozen in liquid nitrogen and stored at −80° C. For immobilized metal affinity chromatography (IMAC)-based protein purification, resuspended bacteria were thawed and disrupted using a French Press (APV 2000, APV Manufacturing, Bydgoszcz, Poland) at 1,000 bar. Following clarification of the lysate by centrifugation at 30,000 g for 1 h, the supernatant was loaded onto an equilibrated Ni-NTA column (Qiagen, Hilden, Germany, cat. no. 30430). Next, the column was washed with 15 column volumes (CV) of wash buffer (50 mM NaH₂PO₄, 300 mM NaCl, 20 mM imidazole, 0.5 mM TCEP, pH 8.0) and the purified protein was eluted in 4 CV of elution buffer (50 mM NaH₂PO₄, 300 mM NaCl, 250 mM imidazole, 0.5 mM TCEP, pH 8.0). Afterwards, the buffer was exchanged to PBS (2.7 mM KCl, 1.5 mM KH₂PO₄, 8.1 mM Na₂HPO₄, 137 mM NaCl, pH 7.4) supplemented with 0.5 mM TCEP by ultrafiltration (PES membrane, 10 kDa MWCO) and the protein (˜10 mg ml⁻¹) was shock frozen in aliquots in liquid nitrogen and stored at −80° C. Before the cell experiments, proteins were thawed, diluted 1:10 in PBS supplemented with 0.5 mM TCEP and used on the same day. The protein mVenus-PIF6 was produced from plasmid pMH302 in E. coli BL21 Star (DE3)pLysS (Thermo Fisher Scientific, cat. no. C602003) at 30° C. for 5 h, purified by IMAC using buffers without TCEP followed by a buffer exchange to PBS as described above. PhyB was produced from plasmid pMH1105 in E. coli and purified by IMAC as described previously. FRB-mCherry-DARPin was produced from plasmid pMH212 in E. coli and purified by IMAC as described previously. Afterwards, the buffer of PhyB and FRB-mCherry-DARPin was exchanged to PBS supplemented with 0.5 mM TCEP as described above.

Protein Characterization

The identity and purity of the proteins was analyzed by SDS-PAGE followed by Zn²⁺-staining of the chromophore PCB (incubation in 1 mM zinc acetate for 10 min followed by imaging of fluorescence under UV light (312 nm) using an agarose gel documentation system (Intas, Gottingen, Germany)) and by Coomassie staining of proteins. As protein size standard, PageRuler prestained protein ladder (Thermo Fisher Scientific, cat. no. 26616) or Pierce prestained protein MW marker (Thermo Fisher Scientific, cat. no. 26612) was used. Protein concentration was determined by Bradford assay (Bio-Rad, Hercules, CA, cat. no. 500-0006) using bovine serum albumin (BSA, Sigma-Aldrich, St. Louis, MO, cat. no. 05479) as standard. The absorbance spectra were acquired with an Infinite M200 Pro microplate reader (Tecan, Mannedorf, Switzerland). The interaction of PhyB-DARPin_{EGFR} and GFP-PIF6 was analyzed by size exclusion chromatography on a light-protected Superdex 200 10/300 GL column (GE Healthcare, Freiburg, Germany, cat. no. 17-5175-01) connected to an Äkta Explorer FPLC system (GE Healthcare) using PBS as running buffer at a flow rate of 0.5 ml min⁻¹. The column was calibrated with a gel filtration standard (Bio-Rad, cat. no. 151-1901).

Cell Culture

A-431 (human epidermoid carcinoma, DSMZ, Braunschweig, Germany, cat. no. ACC 91, cell line identity verified by short tandem repeat (STR) profiling), A549 (human lung carcinoma, CLS, Eppelheim, Germany, cat. no. 300114), HeLa (human cervix adenocarcinoma, ATCC, Manassas, VA, cat. no. CCL-2), MDA-MB-231 (human breast adenocarcinoma, obtained from Signalling Factory Core Facility, University of Freiburg, Germany, cell line identity verified by STR profiling) and HEK-293T (human embryonic kidney, DSMZ, cat. no. ACC 635) cells were cultivated in DMEM complete medium (DMEM (PAN Biotech, Aidenbach, Germany, cat. no. P04-03550) supplemented with 10% (v/v) fetal calf serum (FCS, PAN Biotech, cat. no. P30-3602), 100 U ml⁻¹ penicillin and 100 μg ml⁻¹ streptomycin). SK-OV-3 cells (human ovary adenocarcinoma, ATCC, cat. no. HTB-77) were maintained in McCoy's 5A medium (Sigma-Aldrich, cat. no. M8403) supplemented with 10% (v/v) FCS, 2 mM L-glutamine (Thermo Fisher Scientific, cat. no. 25030-024), 100 U ml⁻¹ penicillin and 100 μg ml⁻¹ streptomycin. MDA-MB-453 cells (human breast metastatic carcinoma, ATCC, cat. no. HTB-131) were cultivated in RPMI 1640 medium (Thermo Fisher Scientific, cat. no. 61870-010) supplemented with 10% (v/v) FCS, 100 U ml⁻¹ penicillin and 100 μg ml⁻¹ streptomycin. CHO-K1 (Chinese hamster ovary, DSMZ, cat. no. ACC 110) cells were maintained in HTS medium (Cell Culture Technologies, Gravesano, Switzerland, cat. no. CHTS) supplemented with 10% (v/v) FCS, 2 mM L-glutamine, 100 U ml⁻¹ penicillin and 100 μg ml⁻¹ streptomycin. All cells were cultivated at 37° C. in a humidified atmosphere containing 5% CO₂ and passaged upon reaching a confluence of ˜80%.

Generation of Stable HEK-293T-SpyCatcher Cell Line

To generate HEK-293T cells stably expressing SpyCatcher-MHCI-moxBFP, the inventors used lentiviral transduction as described previously. Briefly, HEK-293T cells were transfected with the lentiviral packaging plasmid pCMV dR8.74, the envelope plasmid pMD2 vsvG (both kind gifts from Didier Trono) and the transfer plasmid pOSY115 by polyethylenimine-mediated transfection. 48 h post transfection, lentiviral particle-containing HEK-293T supernatant was harvested, filtered using a 0.45 μm filter and concentrated by centrifugation through a 20% (w/v) sucrose cushion in PBS supplemented with 1 mM EDTA at 10,000 g at 4° C. for 4 h. After centrifugation, the supernatant was discarded and the viral particles were resuspended in medium using 1% of the original cell supernatant volume. HEK-293T cells were transduced with increasing amounts of concentrated lentiviral particles to obtain ˜95% transduced cells. Transduction (BFP) and surface expression (anti-HA antibody) were analyzed after 48 h by flow cytometry.

Analysis of Protein Binding to Cells

To analyze the binding of PhyB and PhyB-DARPin_{EGFR} to A-431 cells, A-431 cells were detached with trypsin/EDTA solution (PAN Biotech, cat. no. P10-023500) and washed once with DMEM complete. Afterwards, 8×10⁵ cells ml⁻¹ were incubated with 1 μM of PhyB or PhyB-DARPin_{EGFR} in DMEM complete for 2 h at 37° C. in the dark. Next, cells were washed with PBS, resuspended in PBS supplemented with 2% (v/v) FCS and analyzed for PhyB fluorescence with a Gallios flow cytometer (Beckman Coulter, Brea, CA) using a 638 nm laser for excitation and a 660/20 nm bandpass filter for emission.

To analyze the recruitment of mVenus-PIF6 to the cell surface by flow cytometry, 5×10⁴ A-431 cells were seeded per well of a 24-well plate. After 24 h, cells were incubated with the indicated concentrations of proteins in 500 μl PBS supplemented with 1% (w/v) BSA under the indicated illumination condition. After washing with PBS, cells were trypsinized and mVenus fluorescence was analyzed with a Gallios flow cytometer using a 488 nm laser for excitation and a 525/40 nm bandpass filter for emission. To analyze the recruitment of mVenus-PIF6 to the cell surface and EGFR/PhyB-mCherry-SpyTag internalization by microscopy, 7×10⁴ A-431 cells or 9×10⁴ HEK-293T-SpyCatcher cells were seeded per well of a 24-well plate each containing a coverslip (previously coated with 50 μg ml⁻¹ collagen I (Thermo Fisher Scientific, cat. no. A1048301) in 20 mM acetic acid for 3 h). After 24 h, cells were incubated as indicated with the different proteins and after two washing steps with PBS fixed with 4% (w/v) paraformaldehyde (PFA) in PBS for 20 min. Next, nuclei were stained with TO-PRO-3 (Thermo Fisher Scientific, cat. no. T3605, 0.5 μM in PBS for 15 min) and cells were imaged on a Zeiss LSM 880 laser scanning confocal microscope (Zeiss, Oberkochen, Germany) using a 63× Plan-Apochromat objective (NA: 1.4). mVenus, mCherry and TO-PRO-3 were excited with a 514, 561 and 633 nm laser and detected between 517-543 nm, 570-597 nm and 643-720 nm, respectively (pinhole was adjusted to image a 1.0 μm section for each channel).

AAV Vector Production and Purification

AAV vectors were produced using the adenovirus helper-free packaging system. For production of OptoAAVs, HEK-293T cells were transfected with the plasmids pMH303, pRCVP2koA (kind gift from Hildegard Büning), pHelper (Cell Biolabs, San Diego, CA, cat. no. VPK-402) and the self-complementary vector plasmid pCMVgfp or pCMVmScarlet (both plasmids were kind gifts from Dirk Grimm) in an equimolar ratio. For production of wild type AAV-2 vectors encoding GFP, cells were transfected with the plasmids pAAV-RC2 (Cell Biolabs, cat. no. VPK-402), pHelper and the self-complementary vector plasmid pCMVgfp in an equimolar ratio. Briefly, 8×10⁶ HEK-293T cells were seeded per 15 cm cell culture dish and after 24 h cells were transfected with 60 μg total plasmid DNA mixed with 200 μg polyethylenimine (PEI, MW 25,000) in 3 ml OptiMEM (Thermo Fisher Scientific, cat. no. 22600-134). After 72 h, cells were scraped from the cell culture plates, pelleted by centrifugation (400 g for 15 min), washed once with PBS and resuspended in virus lysis solution (50 mM Tris-HCl, 150 mM NaCl, pH 8.5; 500 μl per 15 cm dish). After lysing the cells by five freeze-thaw cycles, the lysate was incubated with benzonase (50 U ml⁻¹, Merck Millipore, Darmstadt, Germany, cat. no. 70664-3) at 37° C. for 1 h before cell debris were removed by centrifugation at 4,000 g for 15 min. Finally, AAVs were purified from the supernatant by discontinuous iodixanol density gradient centrifugation. Centrifugation was performed for 2 h at 171,000 g and 4° C. using the Optima L-90 K ultracentrifuge equipped with a Type 70.1 Ti fixed-angle rotor (Beckman Coulter). OptoAAVs from 10 plates were purified per Quick-Seal polypropylene tube (Beckman Coulter, cat. no. 342413). After centrifugation, the 40% iodixanol fraction containing the OptoAAVs or wt AAVs was aliquoted, shock-frozen in liquid nitrogen and stored at −80° C.

AAV Characterization and Quantification

Purified AAV vectors were analyzed by Western Blotting against the viral capsid proteins. To this aim, iodixanol-purified AAVs were mixed with SDS loading buffer (final concentration: 62 mM Tris-HCl (pH 6.8), 10% (v/v) glycerol, 2% (w/v) sodium dodecyl sulfate (SDS), 2.5% (v/v) 2-mercaptoethanol, 0.01% (w/v) bromophenol blue sodium salt) and incubated at 95° C. for 5 min. After separating the samples by SDS-PAGE, the proteins were transferred onto a PVDF membrane and the membrane was blocked with blocking buffer (PBS containing 3% (w/v) milk powder) at 4° C. overnight. Following incubation of the membrane for 2 h with a hybridoma cell supernatant containing AAV-2 specific B1 antibody (kind gift from Dirk Grimm) diluted 1:10 in blocking buffer, the membrane was washed with PBS-T (PBS supplemented with 0.05% (v/v) Tween 20) and incubated for 1 h with secondary anti-mouse HRP-conjugated antibody (GE Healthcare, cat. no. NA931) diluted 1:2,000 in blocking buffer. After washing the membrane with PBS-T, ECL Prime Western blotting detection reagent (GE Healthcare, cat. no. RPN2232) was added and chemiluminescence was imaged with the ImageQuant LAS 4000mini system (GE Healthcare). In agreement with previous studies, the mutations R585A and R588A resulted in reduced mobility of the viral capsid proteins in SDS-PAGE.

The genomic titer of the purified AAVs was determined by quantitative real-time PCR (qPCR). Dilution series of the AAVs and of the vector plasmid pCMVgfp as standard were prepared and a 134 bp fragment was amplified from the CMV promoter using oligonucleotides 5′-TGCCCAGTACATGACCTTATGG-3′ and 5′-GAAATCCCCGTGAGTCAAACC-3′ and the ABsolute qPCR SYBR Green ROX mix (Thermo Fisher Scientific, cat. no. AB1163A). The qPCR was performed on a CFX384 thermocycler (Bio-Rad) using the following temperature protocol: 10 min: 95° C. followed by 40 cycles of 15 s at 95° C., 5 s at 67° C., and 10 s at 72° C.

Binding of AAVs to PhyB Beads

To analyze the accessibility and functionality of PIF6 on OptoAAVs, Ni-NTA agarose beads (Qiagen, cat. no. 30430) were washed with beads buffer (PBS supplemented with 0.01% (w/v) BSA) and incubated for 30 min with 3 mg ml⁻¹ PhyB (40 μl Ni-NTA agarose beads and 740 μg PhyB per experiment). After washing with beads buffer, the PhyB beads were incubated with untreated or heated (62.5° C., 10 min) OptoAAVs_{GFP} (10 μl Opto-AAV_{GFP} (1.7×10¹¹ gc ml⁻¹) and 40 μl PhyB beads per experiment) diluted in beads buffer for 1 h under 660 nm light illumination. As control, OptoAAVs_{GFP} were incubated at the same concentration without beads. After centrifugation (1,600 g, 1 min) to pellet the beads, the supernatants were analyzed for OptoAAV_{GFP} by Western blotting using the B1 antibody.

Analysis of EGFR Activation

To analyze EGFR activation by Western Blotting, 6.5×10⁴ A-431 cells were seeded per well of a 24-well plate. After 24 h, the medium was exchanged to starvation medium (DMEM complete without FCS) and after cultivation for another 24 h the cells were incubated as indicated (recombinant human EGF was purchased from Sigma-Aldrich, cat. no. E9644). Following a washing step with PBS, cells were incubated with 100 μl of lysis buffer (20 mM Tris-HCl (pH 7.5), 1 mM EDTA, 100 mM NaCl, 0.5% (v/v) Triton X-100, 0.1% (w/v) SDS, protease inhibitor (complete protease inhibitor cocktail tablets (Roche, Basel, Switzerland, cat. no. 04693116001)), 10 mM β-glycerophosphate, 50 mM sodium fluoride, 1 mM sodium orthovanadate, 10 mM sodium pyrophosphate) for 10 min on ice. Next, the plate was incubated for 30 min at −20° C. and after subsequent thawing on ice, the lysed cells were transferred into microcentrifuge tubes and centrifuged at 10,000 g at 4° C. for 10 min. Afterwards, the supernatants were mixed with SDS loading buffer and incubated at 95° C. for 5 min. The samples were separated by SDS-PAGE and analyzed by Western Blotting as described above for the AAV samples using the following buffers and antibodies: Washing buffer: TBS-T (TBS (50 mM Tris-HCl, 150 mM NaCl, pH 7.4) with 0.1% (v/v) Tween-20); Blocking buffer: TBS-T with 5% (w/v) BSA; Primary antibodies (diluted 1:1,000 in blocking buffer, incubation at 4° C. overnight): EGFR (Cell Signaling Technology (CST), Danvers, MA, cat. no. 4267), pEGFR (Tyr1068, CST, cat. no. 3777), Erk1/2 (CST, cat. no. 9102), pErk1/2 (Thr202/Tyr204, CST, cat. no. 9101), GAPDH (CST, cat. no. 5174); Secondary antibody (diluted 1:3,000 in blocking buffer, 1 h incubation): anti-rabbit IgG, HRP-linked (CST, cat. no. 7074).

Light-Controlled Transduction

If not indicated otherwise, the OptoAAVs were incubated at 62.5° C. for 10 min in a heat block and stored afterwards on ice (up to 6 hours) before used in the experiment. For the light-controlled transduction experiments that were analyzed by flow cytometry, 5,000 cells were seeded per well of a 96 well plate in 100 μl of the corresponding medium. For experiments with the optoPlate-96, cells were seeded in a black 96-well plate with transparent bottom (Greiner, Frickenhausen, Germany, cat. no. 655090). After 24 h, the OptoAAVs were mixed with the indicated PhyB-DARPin protein in the indicated buffer/medium, illuminated for 5 min with 740 nm light and added to the corresponding wells after washing cells once with PBS. After incubation for the indicated period under the indicated illumination condition, wells were washed with PBS and the cells were further incubated in their corresponding medium under the indicated illumination. 48 h after addition of the AAVs to the cells, the wells were washed with PBS and the cells were detached by the addition of 50 μl trypsin/EDTA solution per well. Afterwards, 200 μl PBS supplemented with 5% (v/v) FCS was added to each well and the cells were analyzed for transgene expression using an Attune NxT flow cytometer (Thermo Fisher Scientific). BFP, GFP/CFSE, mScarlet and eFluor670 were excited with a 405, 488, 561 and 637 nm laser and detected using a 440/50, 530/30 nm, 620/15 and 670/14 nm emission filter, respectively. Autofluorescence of the cells was measured in the unused BFP or eFluor670 channel. Flow cytometry data was analyzed with FlowJo (v10.6.1, Becton, Dickinson and Company, Franklin Lakes, NJ) and the gating strategy used throughout this study is depicted in FIG. S6. For experiments with the HEK-293T-SpyCatcher cell line, only BFP-positive cells (˜95%) were used for the transduction analysis.

For spatially resolved transduction experiments using a photomask, 1×10⁵ or 4×10⁴ A-431 cells were seeded in 750 or 300 μl medium per well of a μ-Slide 4 well (ibidi, Gräfelfing, Germany, cat. no. 80426, FIGS. 3A and B) or μ-Slide 8 well (ibidi, cat. no. 80826, FIGS. S12A and B) chambered coverslip, respectively. After 24 h, cells were incubated as described sequentially with PhyB-DARPin_{EGFR} and OptoAAV under the indicated illumination regime. 24 h after addition of the OptoAAVs to the cells, cells were transferred from 37° C. to 30° C. to reduce proliferation and were incubated for another 24 h. Afterwards, cells were fixed with 4% (w/v) PFA in PBS for 20 min, DAPI stained (1 μg ml⁻¹ in PBS) and imaged on a Zeiss LSM 880 laser scanning confocal microscope using a 10× EC Plan-Neofluar objective (NA: 0.3). DAPI was excited with a 405 nm laser and detected between 417-470 nm. GFP and mScarlet were excited with 488 and 561 nm lasers and detected in lambda scanning mode (GFP: 499-695 nm, bin width: 8.9 nm; mScarlet: 570-695 nm, bin width: 8.9 nm), respectively. Afterwards, GFP and mScarlet signals were separated from autofluorescence by linear unmixing using Zen Black (v2.3 SP1, Zeiss), tiles were stitched using Zen Blue (v3.1, Zeiss) and median filtered in Fiji.

For spatially resolved transduction experiments using a confocal microscope, A-431 cells were stained with 1.5 μM or 5 μM of the cell proliferation dye CFSE (Thermo Fisher Scientific, cat. no. C34554) or eFluor670 (Thermo Fisher Scientific, cat. no. 65-0840-90) according to the manufacturer's instructions, respectively. The stained cells were mixed with unstained cells in a ratio of 1:99 and 1.5×10 4 cells were seeded in 300 μl medium per well of a μ-Slide 8 well grid-500 (ibidi, cat. no. 80826-G500) gridded (with lettered and numbered squares) and chambered coverslip. After 48 h, cells were incubated as described sequentially with PhyB-DARPin_{EGFR} and the OptoAAV_{mScarlet} or OptoAAV_{GFP} within a stage top incubator (Tokai Hit, Fujinomiya, Japan) installed on a Zeiss LSM 880 laser scanning confocal microscope. CFSE/Transmission and eFluor670 images were acquired using a 25× LD LCI Plan Apochromat water objective (NA: 0.8) and a 488 and 633 nm laser, respectively. CFSE and eFluor670 fluorescence was detected at 490-570 nm and 651-740 nm, respectively. During imaging, cells were constantly illuminated with 740 nm light (200 μmol m⁻² s⁻¹). After selecting a single, isolated CFSE or eFluor670 positive cell for light-controlled transduction, the 740 nm light was switched off and the cell was illuminated spatially resolved with the 633 nm laser using the bleaching function of the microscope (pixel dwell time: 0.77 μs, pixel size: 0.554 μm, 633 nm laser intensity: 0.5%, 15 iterations). Afterwards, the samples were processed and imaged as described above for the spatially resolved transduction experiments using photomasks. The grid was plotted into the fluorescence images based on its weak autofluorescence.

Transduction Experiment with T Cells

Peripheral blood mononuclear cells (PBMCs) were isolated from healthy human donors by density gradient centrifugation (Ficoll-Paque). Afterwards, PBMCs were resuspended in RPMI complete medium (RPMI 1640 medium supplemented with 10% (v/v) FCS, 10 mM HEPES (Thermo Fisher Scientific, cat. no. 15630-080), 10 μM sodium pyruvate (Thermo Fisher Scientific, cat. no. 11360-039), lx MEM non-essential amino acids (PAN Biotech, cat. no. P08-32100), 50 U ml⁻¹ penicillin and 50 μg ml⁻¹ streptomycin) supplemented with 500 U ml⁻¹ IL-2 (PeproTech, Hamburg, Germany, cat. no. 200-02) and activated with anti-CD3/CD28 (1 μg ml⁻¹) antibodies. After 72 h, the remaining PBMCs were mostly T cells, which were used for light-controlled transduction using PhyB-DARPincD4 and OptoAAV_{GFP} as indicated. 6 h after addition of the OptoAAVs_{GFP}, the cells were incubated in RPMI complete medium supplemented with 100 U ml⁻¹ IL-2 under the indicated illumination. After 42 h, the cells were stained for CD4 expression by incubation with CD4-V450 antibody (BD Biosciences, San Jose, CA, cat. no. 560345) diluted 1:200 in PBS supplemented with 2% (v/v) FCS at 4° C. for 15 min. After washing, the cells were resuspended in PBS supplemented with 2% (v/v) FCS and analyzed using an Attune NxT flow cytometer as described above. V450 fluorescence and autofluorescence was measured in the BFP and mScarlet channel, respectively. Autofluorescent cells (<1.7%) were excluded from the analysis.

Statistical Analysis

Statistical significance was tested with unpaired two-sided t tests (no assumption of consistent standard deviations) with correction for multiple comparisons (Holm-Sidak method) using GraphPad Prism (v8.4.3, GraphPad Software, San Diego, CA).

Results and Discussion

Design of the OptoAAV System

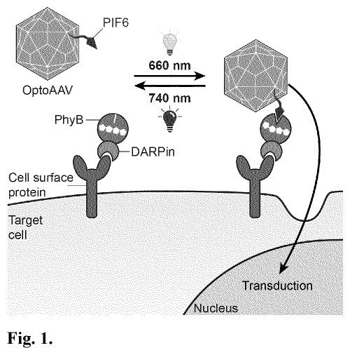

The OptoAAV technology comprises an engineered AAV-2 and a light-responsive adapter protein that mediates selective interaction of the AAV with the target cell (FIG. 1). The viral vector is genetically modified to be blind to its natural cellular receptor (heparan sulfate proteoglycan, HSPG) and to expose the phytochrome interacting factor 6 (PIF6, amino acids 1-100) from A. thaliana on the capsid surface (OptoAAV). The adapter protein consists of phytochrome B (PhyB, amino acids 1-651) of A. thaliana and a designed ankyrin repeat protein (DARPin) specific for a cell surface protein of the target cell (PhyB-DARPin). Upon illumination with red (˜660 nm) light, PhyB of the adapter protein interacts with PIF6 on the viral vector thus recruiting OptoAAV to the cell surface, which results in transduction of the target cell. In contrast, illumination with far-red (˜740 nm) light dissociates the interaction between PhyB and PIF6 and consequently prevents transduction (FIG. 1).

Implementation of the OptoAAV System

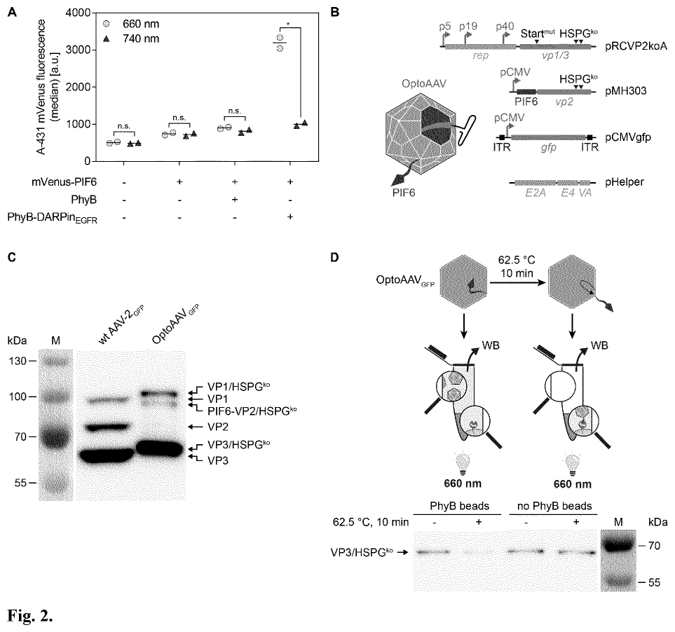

To develop and characterize the OptoAAV system, the inventors selected the DARPin E_01 showing high affinity (K_{D}=0.5 nM) to the human epidermal growth factor receptor (EGFR) that is overexpressed by many tumor cells. This and other DARPins have previously been used for the retargeting of AAV, adenoviral and lentiviral vectors and measles virus either by exposing them on the viral surface or by using them as adapter mediating the interaction between cell and viral vector. The inventors produced the fusion protein of DARPin_{EGFR} and the photosensory domain of PhyB in E. coli and purified it via its hexahistidine tag by immobilized metal ion affinity chromatography (IMAC, PhyB-DARPin_{EGFR}, FIG. S1). The inventors verified PhyB photoswitching by acquiring the absorbance spectra upon 660 and 740 nm illumination (FIG. S2) and validated the light-dependent interaction of PhyB-DARPin_{EGFR} with PIF6 by size exclusion chromatography (FIG. S3). Flow cytometry experiments revealed that PhyB-DARPin_{EGFR} bound specifically via the DARPin_{EGFR} to A-431 cells overexpressing EGFR (FIG. S4). To demonstrate that PhyB-DARPin_{EGFR} can recruit PIF6-tagged molecules to cells in a light-dependent manner, the inventors analyzed light-induced recruitment of an mVenus-PIF6 fusion protein to A-431 cells by flow cytometry (FIG. 2A) and confocal microscopy (FIG. S5). In addition, the microscopy experiment revealed that mVenus-PIF6 was massively internalized after 20 min incubation at 37° C. whereas it remained mainly membrane-localized when cells were incubated on ice. This observation is in line with internalization reported for EGFR.

To display PIF6 on the capsid of AAV-2, the inventors genetically fused the active phytochrome binding (APB) domain of PIF6 to the N-terminus of the viral capsid protein VP2. The inventors placed the coding sequence of the PIF6-VP2 fusion protein under control of a CMV promoter (plasmid pMH303) and prevented expression of native VP2 by deletion of its start codon in the plasmid pRCVP2koA (FIG. 2B). To prevent transduction of cells in a light-independent manner, these plasmids additionally contained the mutations R585A and R588A in the cap genes ablating the natural tropism of AAV-2 for its natural receptor HSPG (FIG. 2B). As model transgene, the inventors selected the fluorescent protein GFP or mScarlet under control of the constitutive CMV promoter encoded on a self-complementary genome. Self-complementary AAV vectors are characterized by a higher transduction efficiency at the expense of only half of the loading capacity. The inventors produced the OptoAAVs using the helper free packaging system in HEK-293T cells and purified assembled AAV capsids by iodixanol gradient ultracentrifugation. The inventors confirmed the incorporation of PIF6-VP2 in OptoAAV_{GFP} and the ablation of native VP2 by Western Blotting against the viral capsid proteins VP1, VP2 and VP3 (FIG. 2C). From one OptoAAV_{GFP} production (10×15 cm dishes), the inventors determined an average genomic titer of (1.6±0.6)×10¹¹ vg (vector genomes). The inventors next analyzed the light-controlled interaction of OptoAAV_{GFP} with PhyB. To this aim, the inventors incubated OptoAAV_{GFP} with PhyB-functionalized agarose beads under 660 nm light and subsequently analyzed the supernatant for unbound viral particles by Western Blotting against the viral capsid proteins (FIG. 2D). Interestingly, OptoAAV_{GFP} did not bind to the PhyB beads suggesting that PIF6 was not accessible or exposed on the capsid surface. As previous studies showed that the N-terminus of VP2 becomes exposed upon limited heat shock, the inventors incubated the OptoAAVs_{GFP} for 10 min at 62.5° C. and repeated the binding experiment to the PhyB beads. Indeed, upon heat treatment, the inventors observed binding of OptoAAV_{GFP} to PhyB (FIG. 2D).

Red Light-Controlled Transduction

After the characterization of the individual components, the inventors tested the ability of the system to transduce A-431 cells in a light-dependent manner. For this, the inventors used OptoAAVs_{GFP} that had been previously incubated for different periods of time at different temperatures (FIG. 3A, FIGS. S6 and S7A). The inventors observed a 5.6-81-fold increase in the percentage of transduced cells at 660 nm compared to 740 nm illumination (low light intensity, both 20 μmol m's⁻¹, equals 0.36 and 0.32 mW cm⁻², respectively). In agreement with the above-described in vitro binding studies (FIG. 2D), heat treatment of OptoAAV_{GFP} resulted in a strong increase of transduced cells (from ˜2% to ˜40%). Based on these data, the inventors exposed PIF6 on OptoAAVs by a 10 min incubation step at 62.5° C. in all further experiments. Transduction experiments with untreated and heated unmodified (wt) AAV-2 vectors revealed that this heat treatment reduced the infectious titer 5.9-fold (FIG. S7B). For OptoAAV, a likely similar loss of infectivity is outcompeted by the gain in light-controlled infectivity caused by the heat-induced exposure of PIF6. Control experiments verified that the adapter protein and OptoAAV_{GFP} are both required for transduction and that the transduction efficiency of unmodified AAV-2 vectors in the presence of the adapter protein is not affected by 660 and 740 nm illumination (FIG. 3B). The percentage of transduced A-431 cells reached up to 65% with increasing OptoAAV_{GFP} titer, while the 660 nm light-induced 37-fold change in transduced cells was not affected by the viral titer. The inventors further demonstrated that the percentage of transduced target cells can precisely be fine-tuned by adjusting the illumination intensity and/or period (FIG. 3C). The inventors observed light-controlled viral transduction over a broad range of adapter protein concentration (0.5-500 nM) with the highest percentage of transduced cells at 50 nM (FIG. S8A). The system was functional in PBS supplemented with BSA or FCS and in different commonly used cell culture media (FIG. S8B). Furthermore, the transduction efficiency increased with the incubation time of the cells with OptoAAV_{GFP} and the adapter protein (FIG. S8C). Although cellular binding of the used DARPin_{EGFR} does not trigger EGFR signaling, multiple AAV-bound PhyB-DARPin_{EGFR} adapter proteins may crosslink and activate EGFR under 660 nm illumination. However, analysis of EGFR and Erk1/2 phosphorylation by Western Blotting did not show any receptor activation by the OptoAAV_{EGFR} system (FIG. S9). Using the PhyB-DARPin_{EGFR} adapter protein, the inventors could further transduce the EGFR-expressing tumor cell lines A549, HeLa and MDA-MB-231 in a light-dependent manner, but not CHO-K1 cells lacking EGFR (FIG. S10).

While EGFR is known to be internalized by endocytosis, the inventors next asked whether active internalization of the cellular target receptor is a prerequisite for the functionality of the OptoAAV system. To this aim, the inventors displayed SpyCatcher on the surface of HEK-293T cells by fusing it to the secretion signal and transmembrane domain of MHC I. Afterwards, the inventors covalently coupled purified PhyB-mCherry-SpyTag to SpyCatcher. The MHC I transmembrane domain has previously been used to anchor proteins stably on the cell membrane and does to the knowledge of the inventors not contain an endocytosis function. Indeed, the inventors observed only minimal internalization of PhyB-mCherry-SpyTag on the engineered HEK-293T by confocal microscopy in comparison to the massive internalization of EGFR on A-431 cells (FIG. 511A). As the inventors were able to transduce the engineered PhyB-displaying HEK-293T cells with OptoAAV in a light-dependent manner with a similar efficiency as for the A-431 cells (FIG. 511B), the inventors suggest that active internalization of the cellular target receptor is not required for the OptoAAV system. The inventors hypothesize that viral uptake is induced upon cell attachment by binding of the OptoAAV to co-receptors such as the AAV receptor (AAVR).

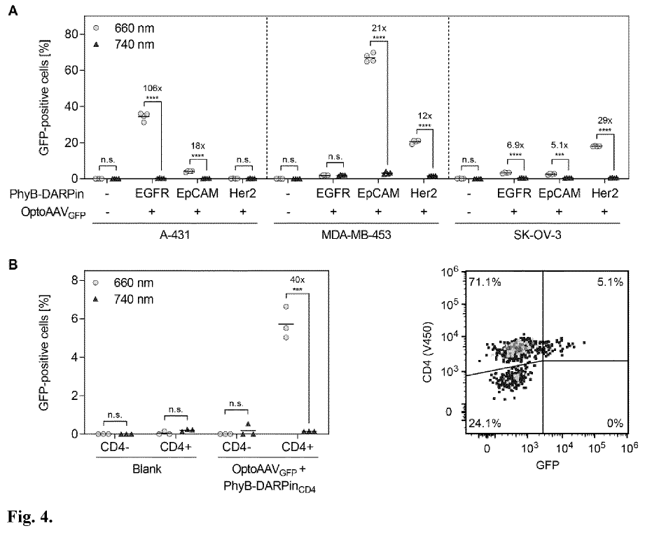

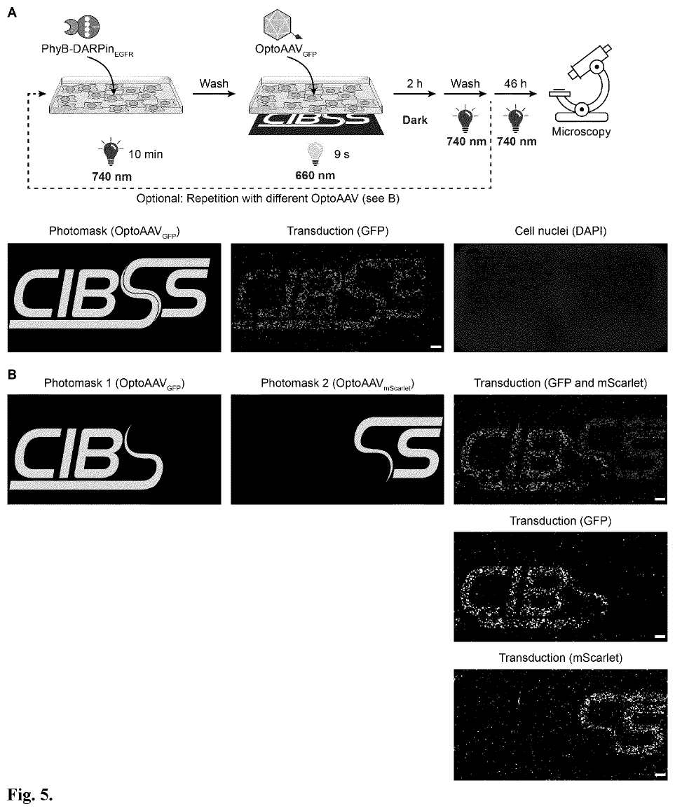

To demonstrate that the OptoAAV system can be expanded to other cell lines with minimal or absent EGFR expression, the inventors modularly exchanged the DARPin_{EGFR} with DARPins specific for EpCAM (DARPin Ec1), Her2/ErbB2 (DARPin 9_29) and CD4 (DARPin D55.2) (FIGS. 51 and S2). Using these adapter proteins, the inventors were able to selectively transduce further cell lines (FIG. 4A) as well as primary human CD4-positive T lymphocytes (FIG. 4B) in a light-dependent manner.

Spatiotemporal Control of Transduction

Next, the inventors aimed at transducing cells in a spatially resolved manner. To this end, the inventors incubated A-431 cells for 10 min with the adapter protein under 740 nm illumination (FIG. 5A). After a washing step to remove unbound adapter protein, the inventors added OptoAAV_{GFP} or OptoAAVm_{Scarlet} and illuminated the cells spatially resolved with 660 nm light for 9 s using a photomask. Following incubation for 2 h in the dark, the inventors washed the cells, incubated them for another 46 h under 740 nm light and visualized the transduced cells by microscopy (FIG. 5A and FIG. S12A). The inventors observed spatially resolved transduction (i.e. expression of the fluorescent proteins) with a spatial resolution in the 100 μm range while non-transduced cells were distributed over the whole wells as seen in the DAPI images. The inventors next tested, whether OptoAAVs encoding different transgenes can be used to sequentially transduce cells in a spatially resolved manner. To this aim, the inventors performed the just described experiment with OptoAAV_{GFP} and photomask 1. Following 2 h incubation in the dark and a subsequent washing step, the inventors repeated the same procedure with OptoAAVm_{Scarlet} and photomask 2 (FIG. 5B and FIGS. S12B and C). This approach enabled the spatially resolved transduction of cells with two different transgenes and can likely be expanded to additional transgenes. In areas illuminated with both photomasks, the inventors observed cells expressing both transgenes, indicating that the OptoAAV system can deliver several transgenes into one target cell (FIG. S12B).

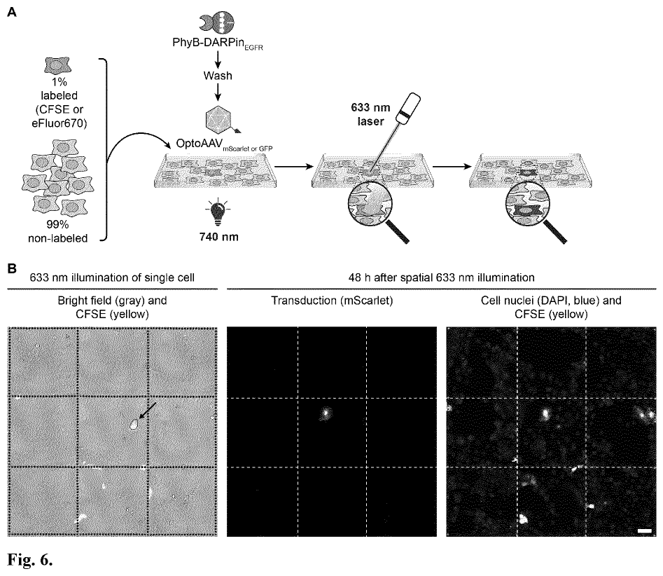

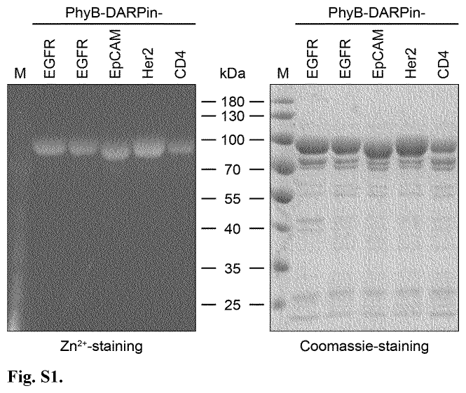

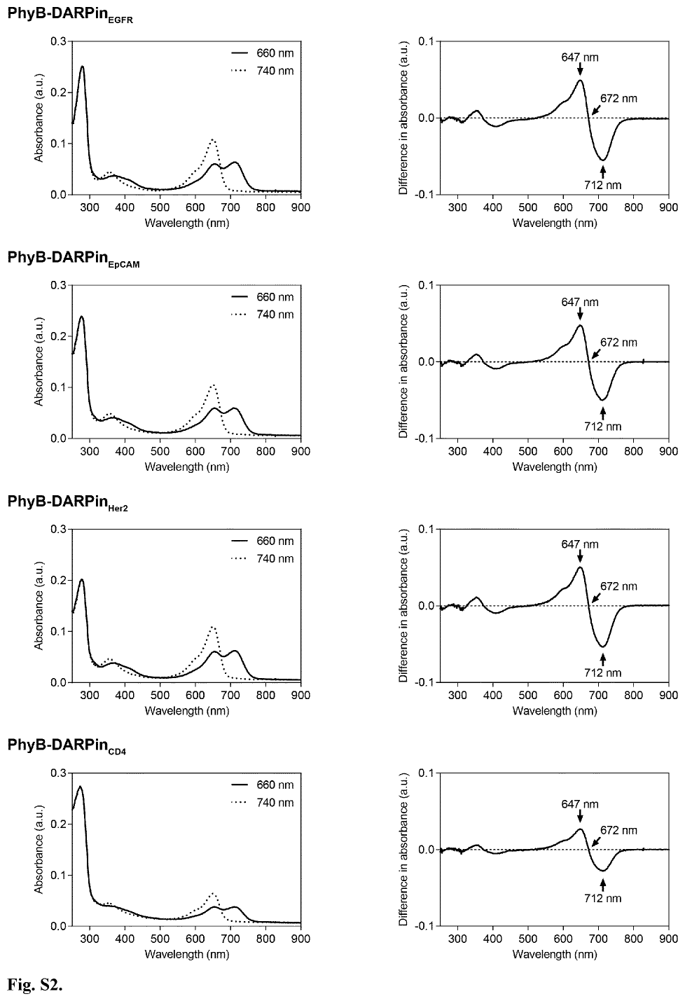

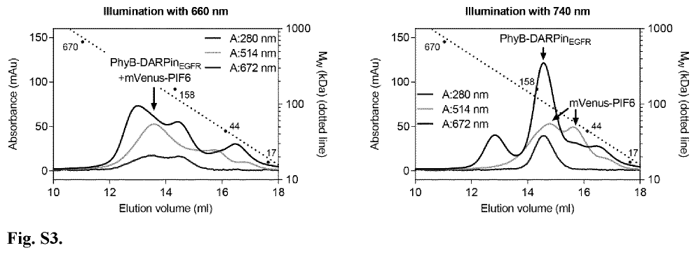

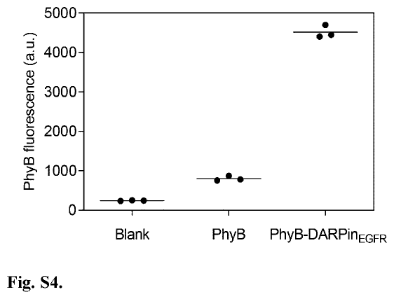

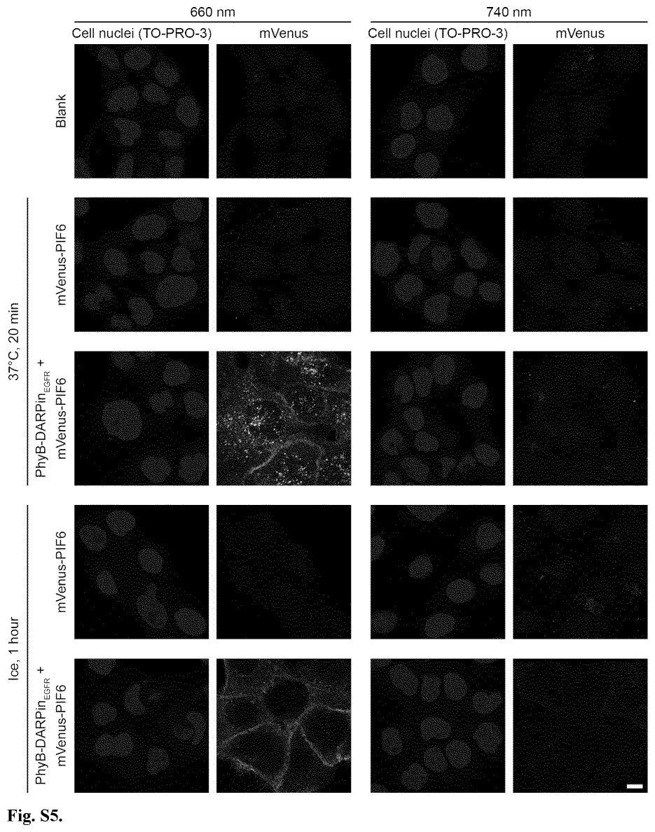

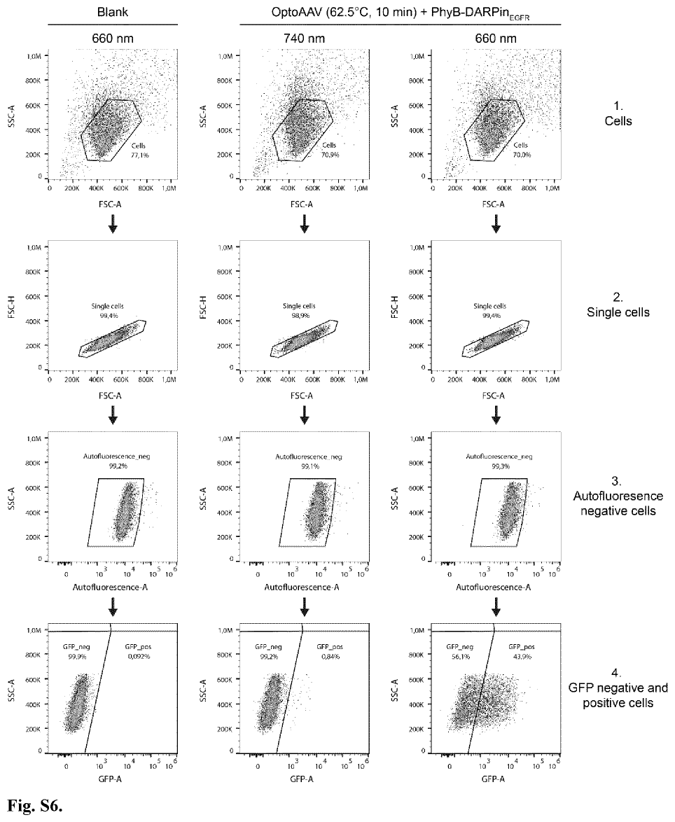

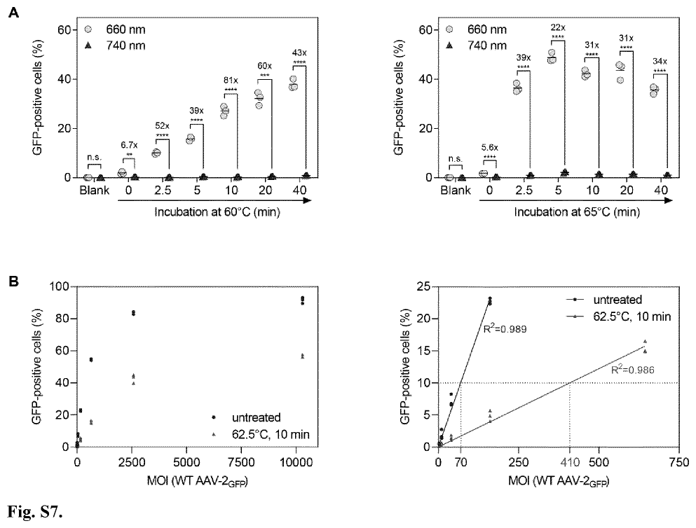

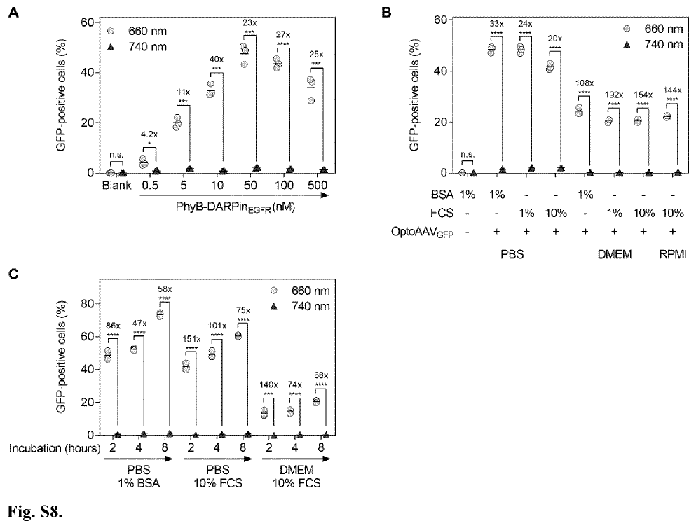

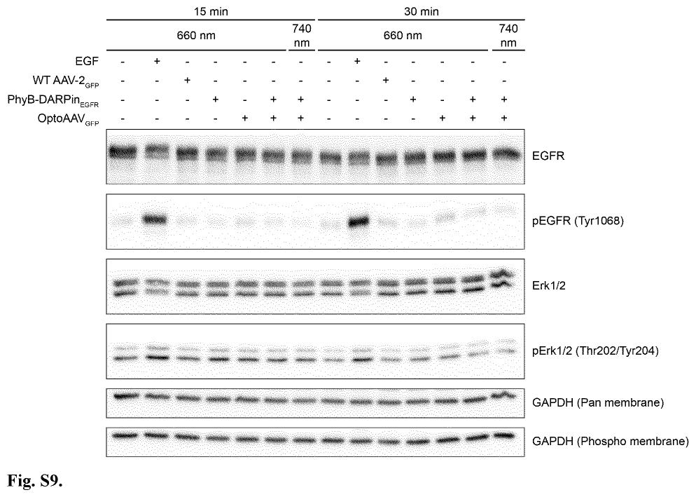

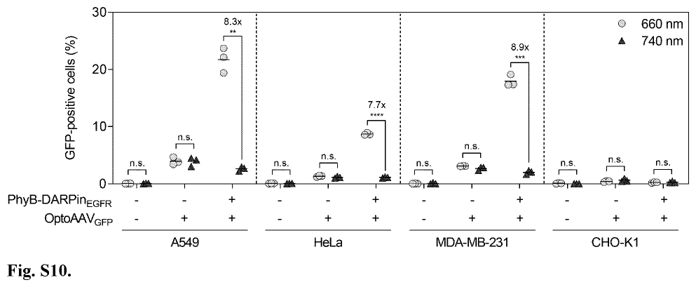

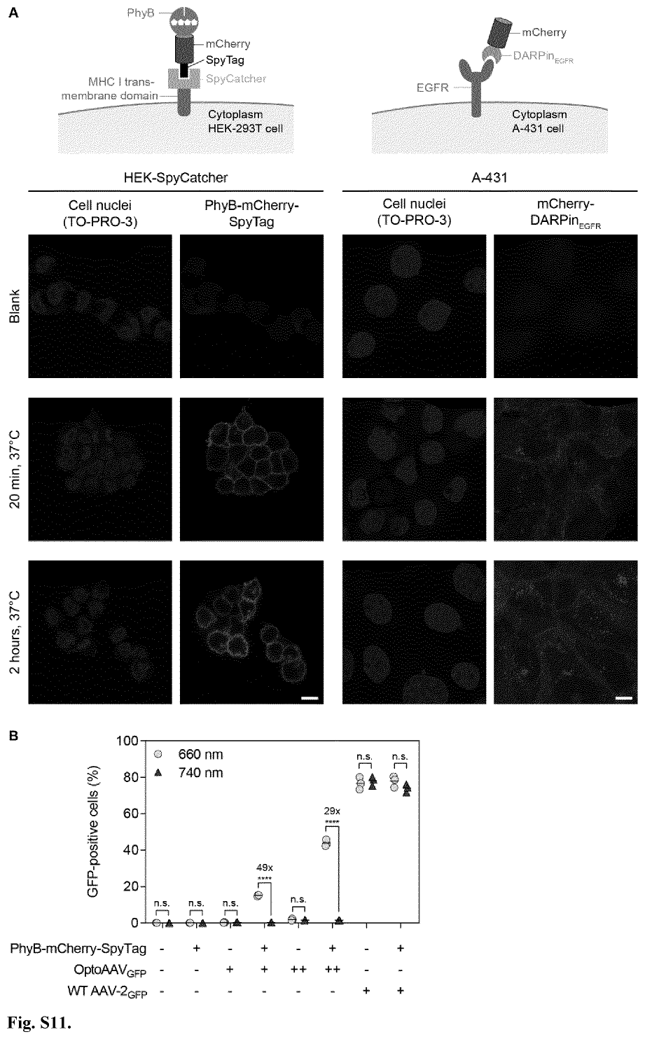

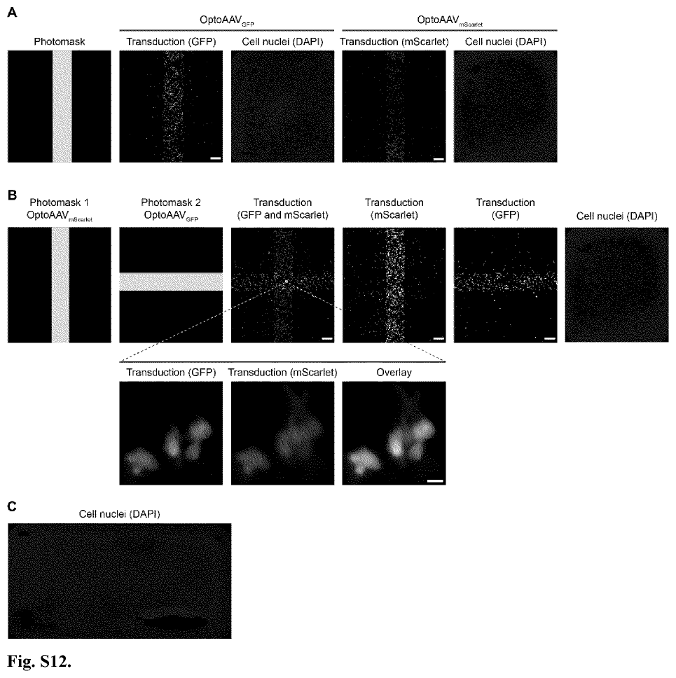

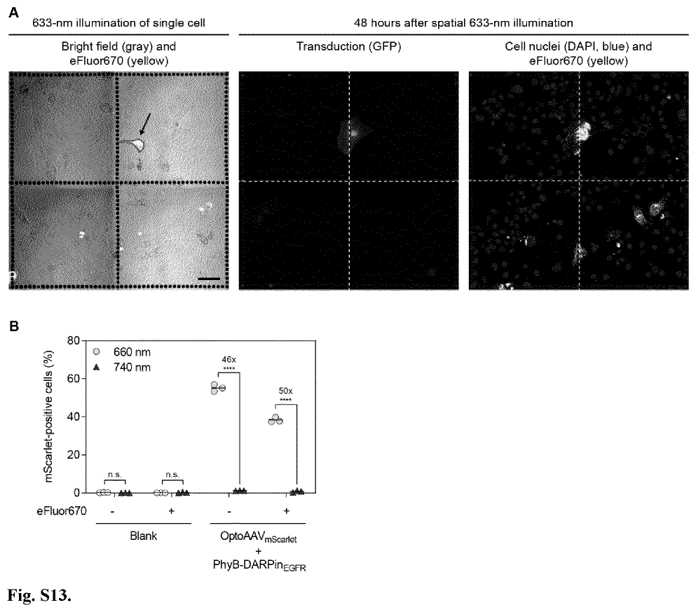

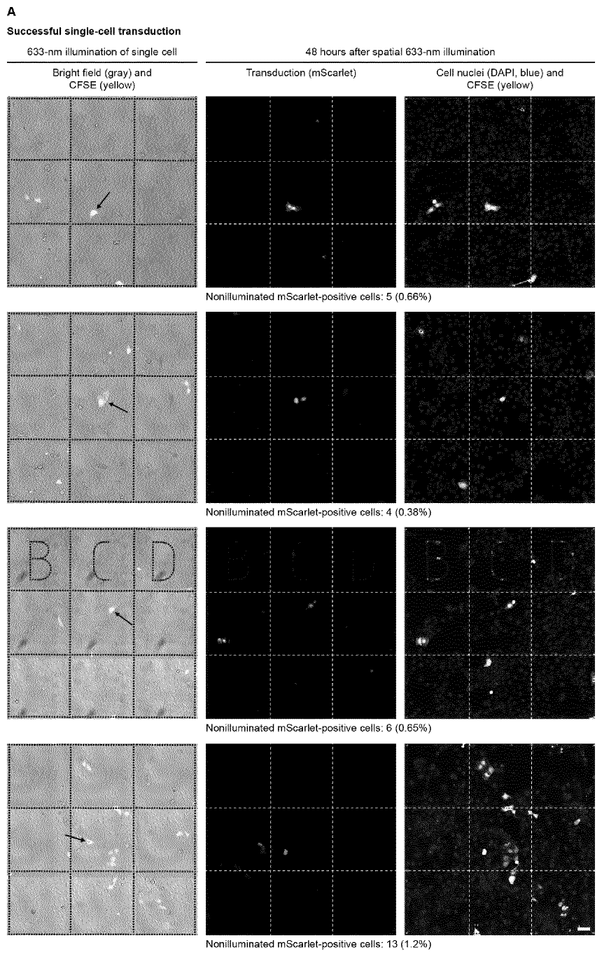

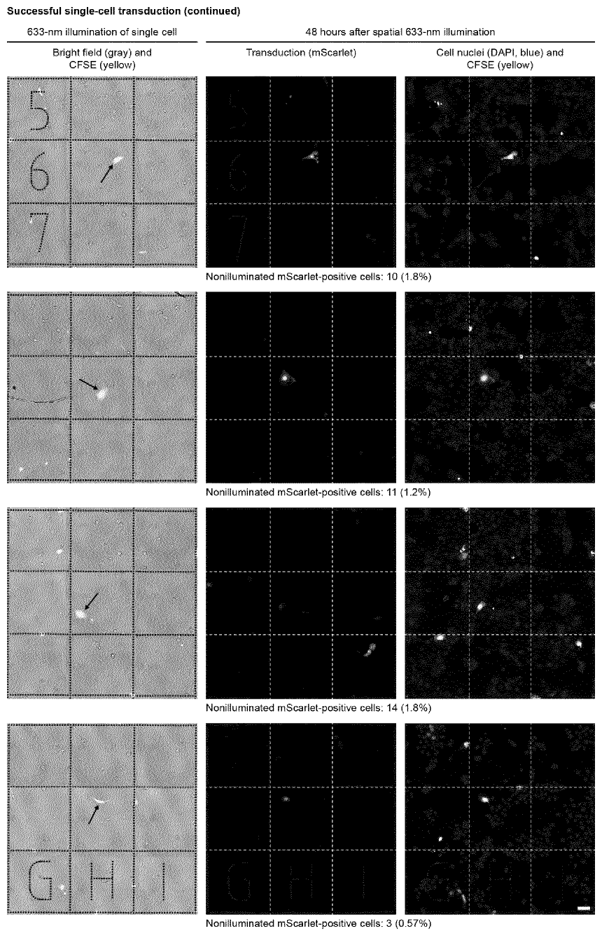

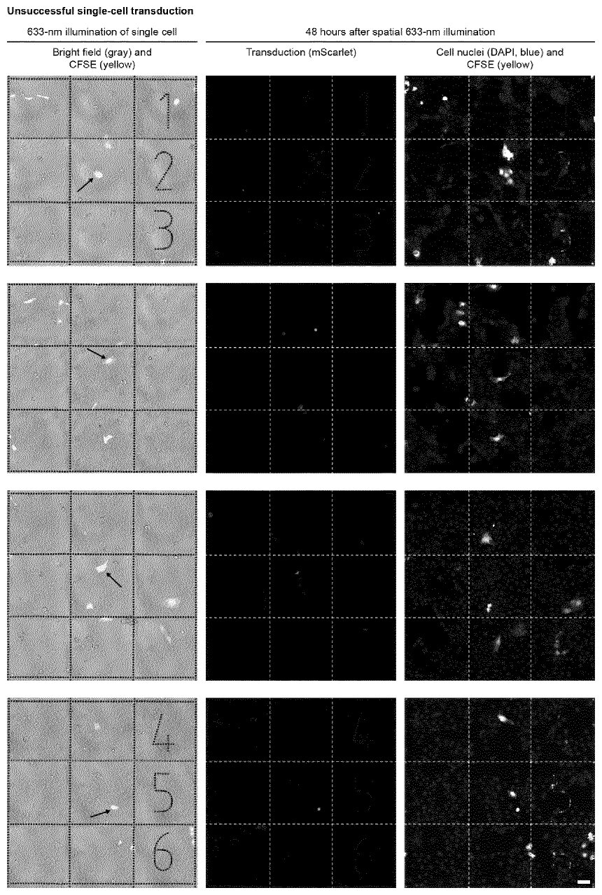

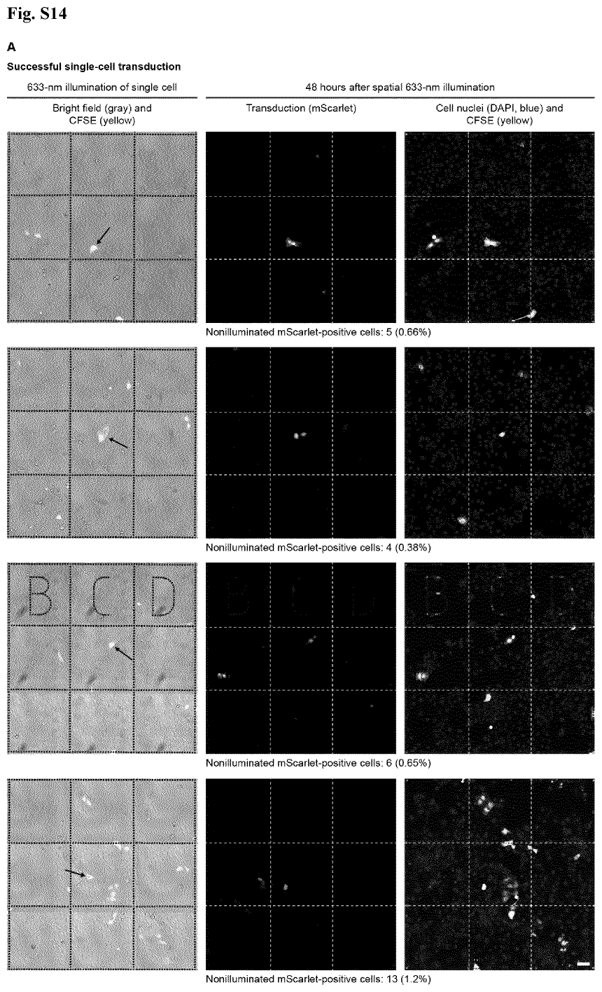

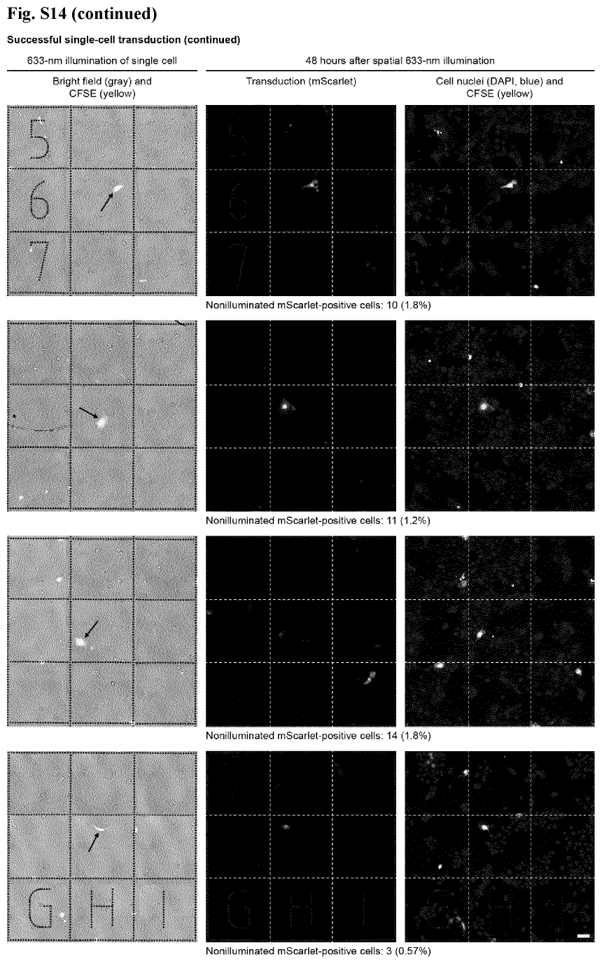

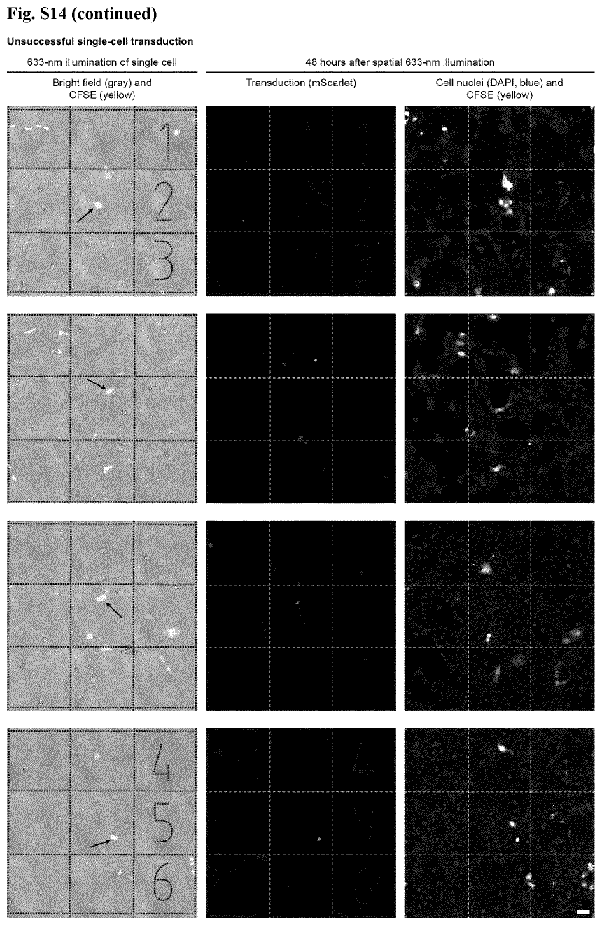

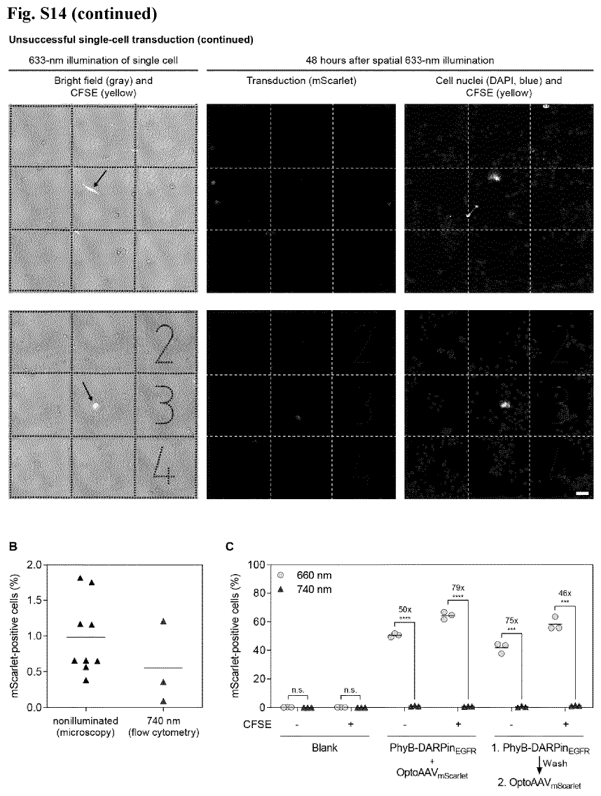

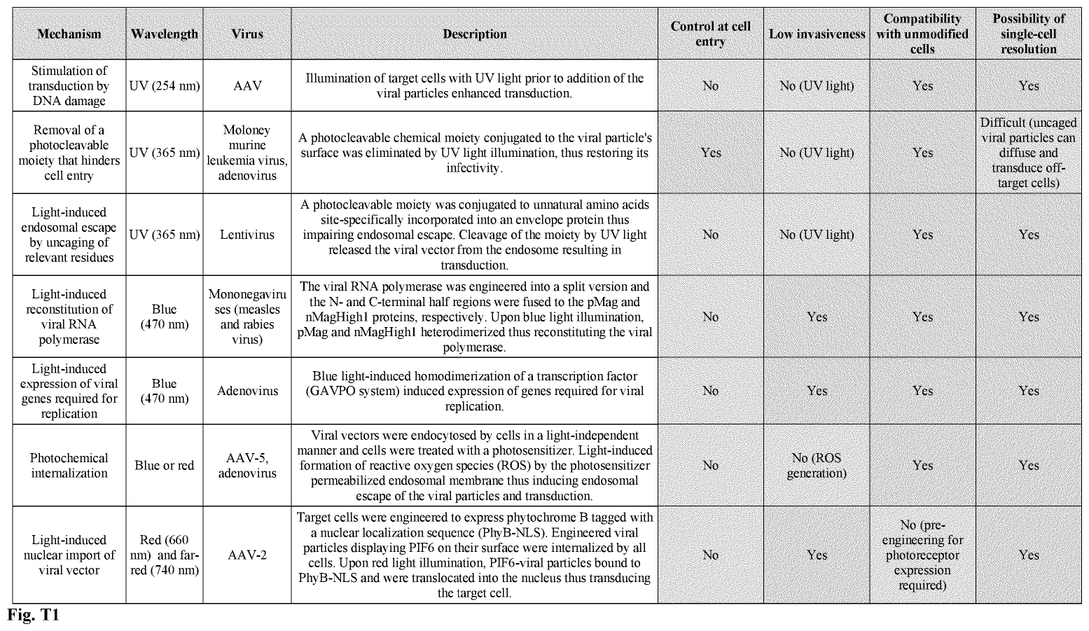

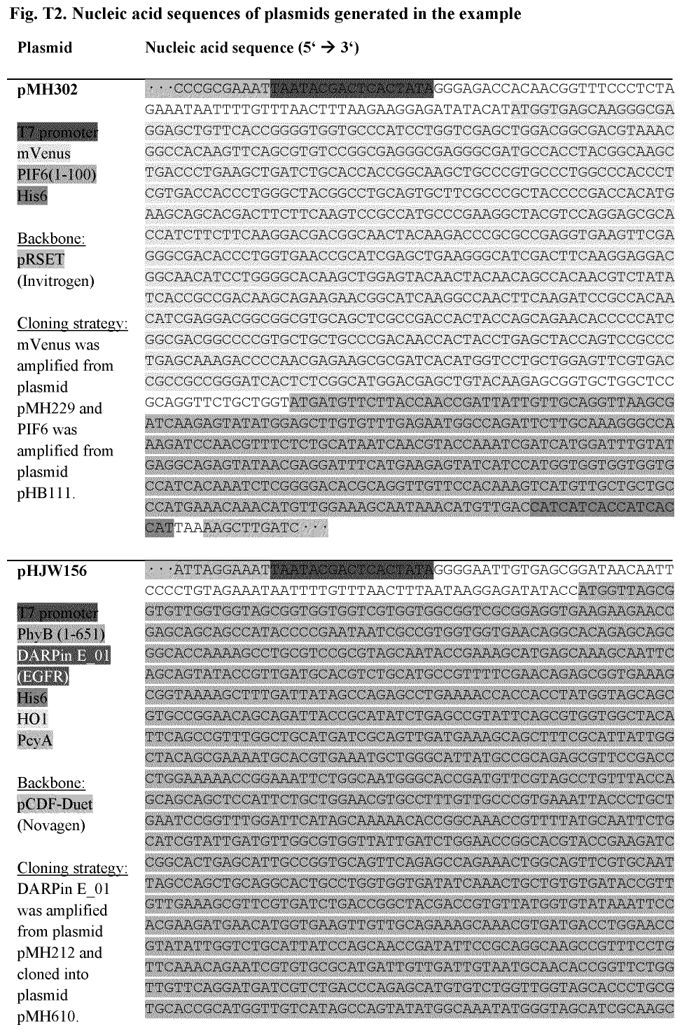

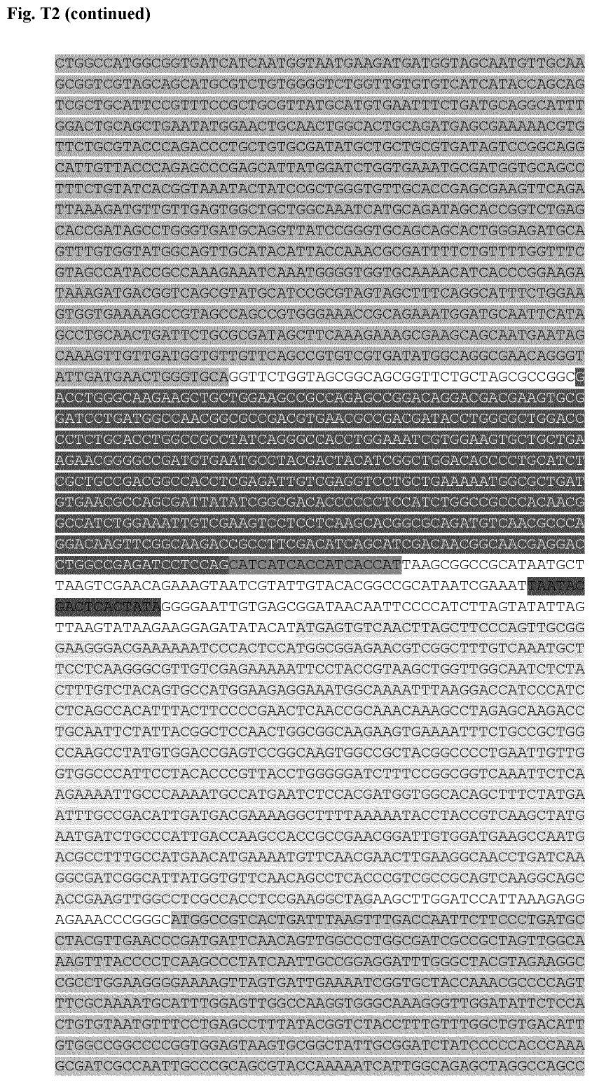

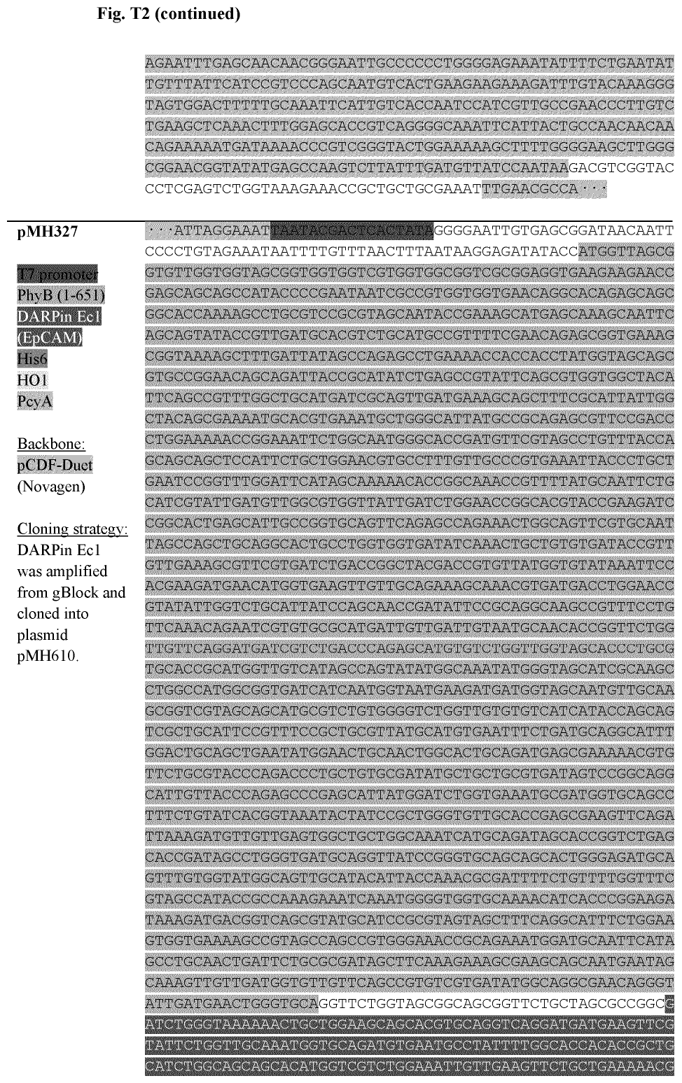

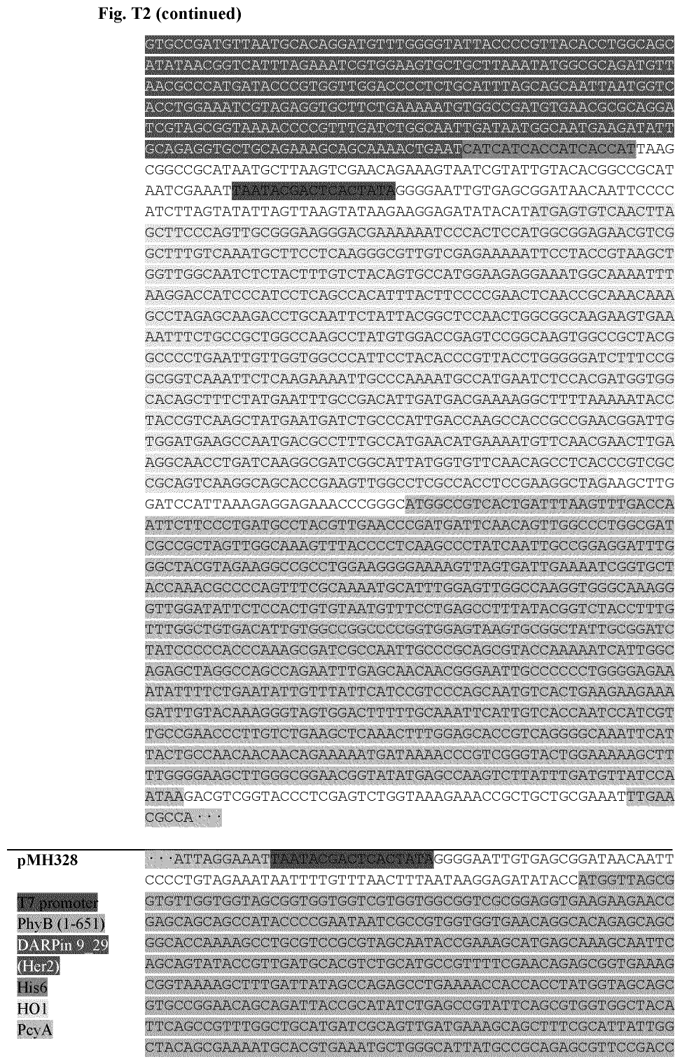

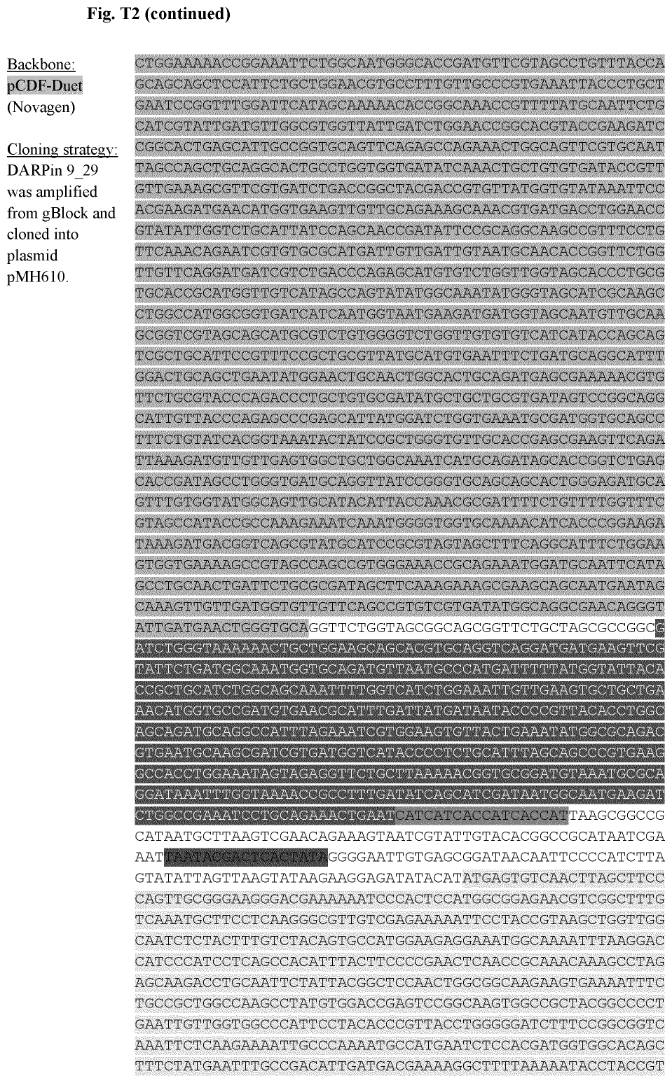

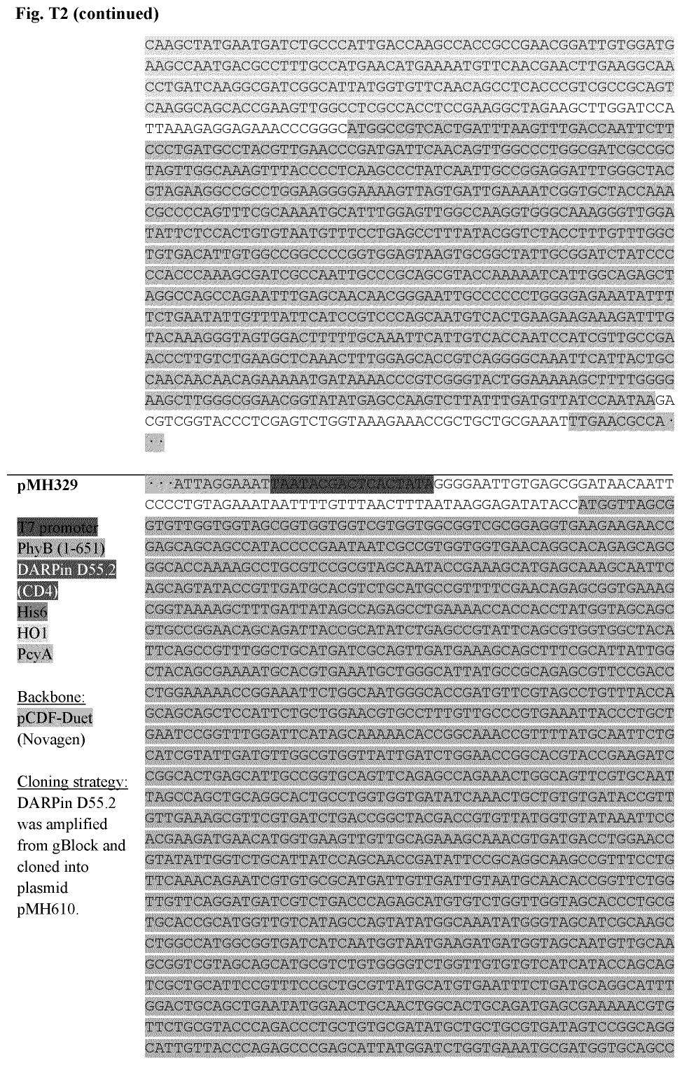

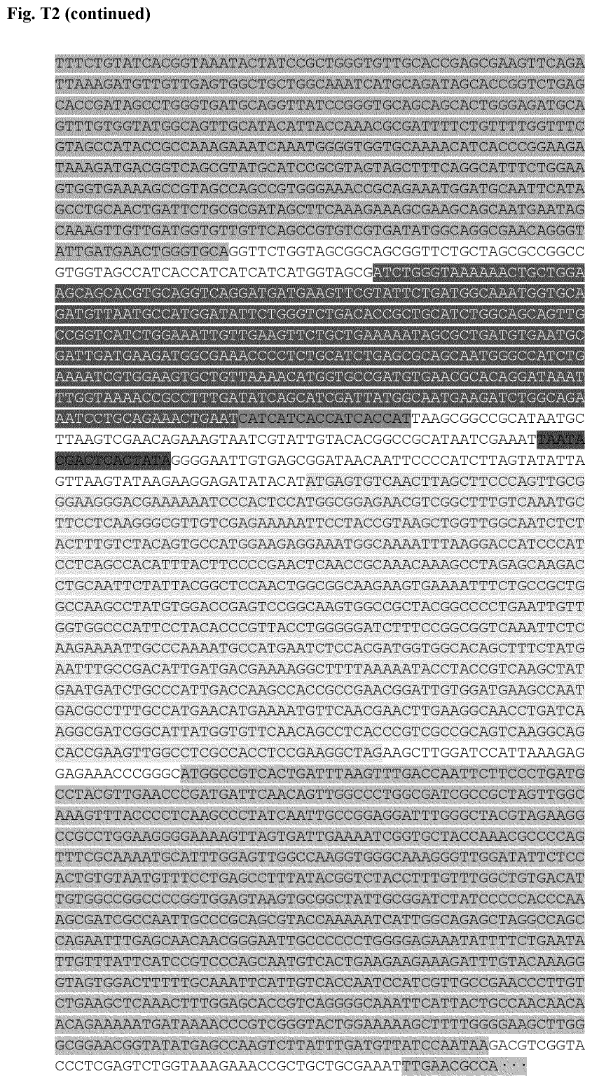

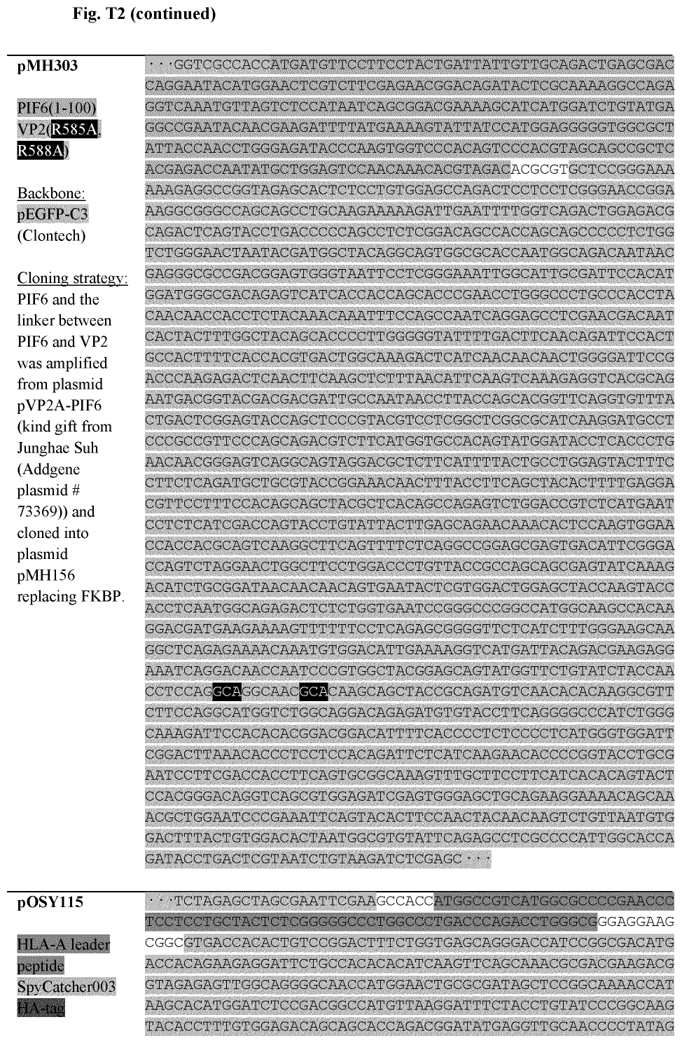

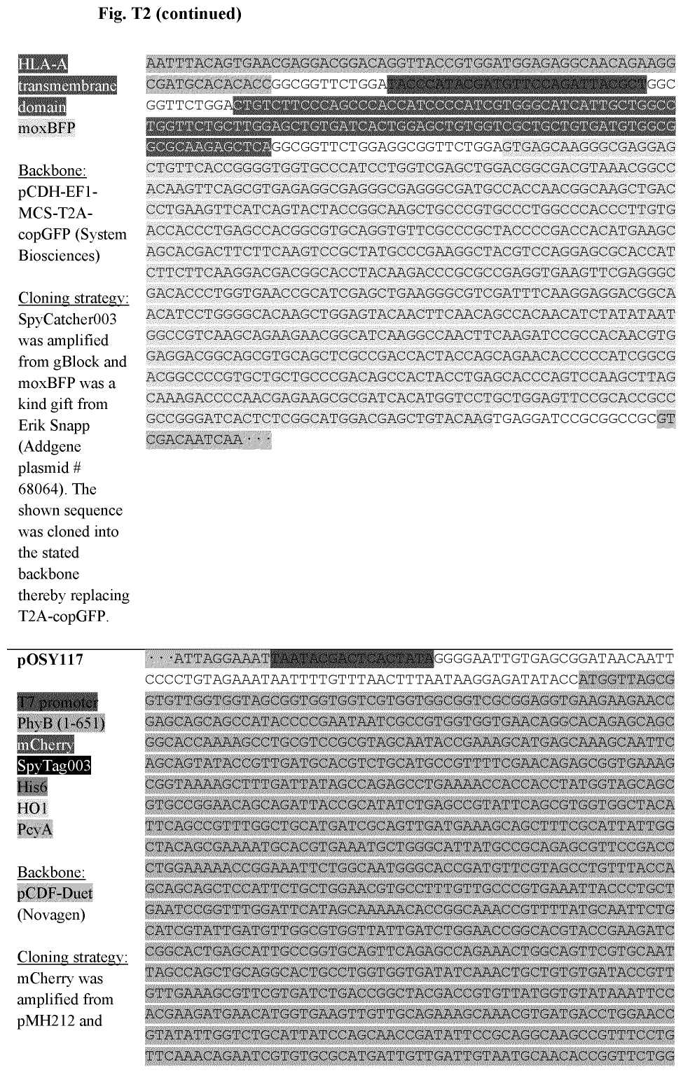

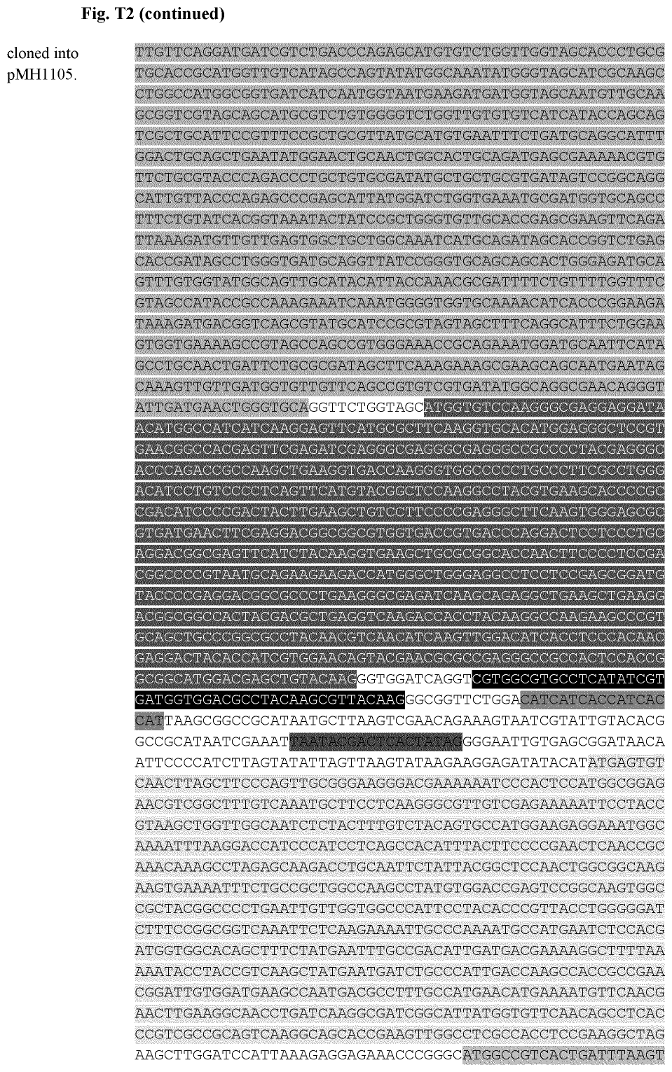

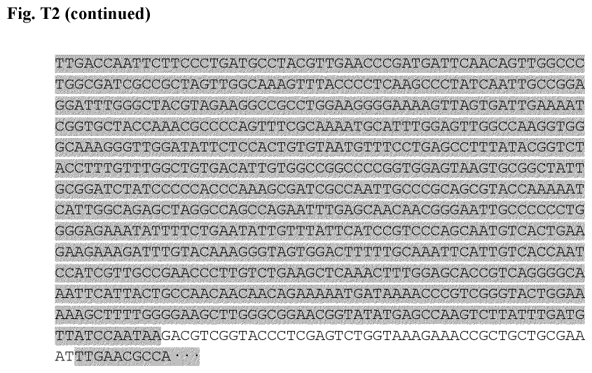

The inventors next extended the spatial resolution to the single-cell level by illumination of one selected cell using a conventional confocal microscope equipped with a 633 nm laser. To track the illuminated cell over the course of the experiment (48 h), the inventors seeded A-431 cells, of which 1% were fluorescently labeled (eFluor670), on a coverslip with a labeled grid (FIG. 6A). Following incubation with the adapter protein and addition of OptoAAV_{GFP} under 740 nm light, the inventors illuminated a single isolated eFluor670-labeled cell with low intensity 633 nm laser light (˜120 ms, FIG. 6A). After 48 h, the inventors analyzed the transduction by microscopy. In 4 out of 17 experiments (24%) the single illuminated eFluor670-labeled cell showed GFP expression (for a representative successful experiment see FIG. S13A). As the success rate was lower than transduction of eFluor670-labeled cells at 660 nm in comparable flow cytometry experiments (38%, FIG. S13B), the inventors hypothesized that absorption of the 633 nm laser by the eFluor670 dye might have reduced photoactivation of PhyB. Therefore, the inventors repeated the experiment with CFSE-labeled cells which have no absorbance at 633 nm and OptoAAVm_{Scarlet}. Indeed, in 9 out of 15 experiments (60%) the inventors were now able to transduce the single illuminated CFSE-labeled cell (FIG. 6B and FIG. S14A), which is in agreement with the transduction rate at 660 nm of comparable flow cytometry experiments (58%, FIG. S14C). From the non-illuminated cells, 1.0% showed mScarlet fluorescence, although mainly with a much lower intensity than the illuminated cell (FIG. S14A). This off-target transduction rate is similar with the one under constant 740 nm illumination in comparable flow cytometry experiments (0.6%, FIG. S14B).

### SUMMARY

In summary, the inventors demonstrated that the OptoAAV technology enables the spatiotemporally resolved and light dose-dependent selective transduction of native cell lines and primary cells using low intensity red light. Due to its modular design, the system can be customized to target cell types of choice by switching to an adapter protein with the desired specificity. OptoAAV intrinsically features a two-factor control for specificity. Successful transduction requires both recognition of the target cell type by the adapter protein and the light stimulus leading to vector binding and internalization. Such AND-type control was shown in previous studies to significantly increase target specificity. Using a conventional confocal microscope, OptoAAV allowed the selective transduction of single cells by local illumination. Such experiments could allow perturbing biological processes at the single-cell level to reconstruct and understand the implications of cell heterogeneity. In addition, the sequential application of different OptoAAVs enabled the selective transduction of cells with different transgenes within the same culture. This feature may be of particular interest when applying OptoAAV for the delivery of genes encoding differentiation factors in tissue engineering for regenerative medicine. Although the inventors only tested the in vitro functionality of OptoAAV so far, it may also be applied for the site-specific in vivo gene delivery in fundamental research or (cancer) gene therapy. Another promising area of application would be in neurosciences. Light-responsive ion channels such as channelrhodopsin (ChR) are routinely used for the light-responsive induction of action potentials in neurons, both in tissue culture and in vivo. In living animals, ChRs are often introduced via AAV-based vectors and the injection of viral vectors can lead to substantial spread of opsin expression, e.g. caused by the backflow of the vector along the injection tract. While this might sometimes be a desired feature, it is often a detrimental complication, hampering conclusions about the role of an activated or deactivated brain area. Using OptoAAV, gene delivery could be targeted with high spatial resolution and selectivity to the cells of interest by (i) choosing an appropriate adapter protein and (ii) by local illumination with red light. For transduction, the same waveguide-based illumination hardware as used for activation of ChR could be used. In an in vivo setting, the spatial resolution and tissue penetration may be further increased by two-photon activation of the OptoAAV system as recently shown for a bacterial phytochrome. Moreover, OptoAAV may be used to obtain further insights into viral cell entry, e.g. by determining required binding times. Due to its modular design, the inventors suggest that the OptoAAV approach could serve as blueprint for rendering further classes of viral vectors light-responsive.

Detailed Description of the FIGS.

FIG. 1. Design and mode of function of the light-controlled viral transduction system. The OptoAAV system comprises (i) an engineered AAV-2 vector displaying PIF6 on its surface and containing mutations that ablate its natural tropism for HSPG (OptoAAV), and (ii) an adapter protein consisting of PhyB and a DARPin selectively binding to a specific cell surface protein. Illumination with 660 nm light induces the interaction of PhyB with PIF6 and thus the recruitment of the engineered OptoAAV to the cell surface resulting in transduction of the target cell.

FIG. 2. Characterization of OptoAAV system components. (A), Light-controlled recruitment of mVenus-PIF6 to cells. A-431 cells were incubated with 100 nM PhyB or PhyB-DARPin_{EGFR} and/or 200 nM mVenus-PIF6 for 20 min under 740 nm or pulsed 660 nm light. Afterwards, cellular mVenus fluorescence was analyzed by flow cytometry. n>2,200 cells/sample. Mean is indicated. n.s. P≥0.05, *P<0.05. (B), Construction of OptoAAVs. OptoAAVs were produced in HEK-293T cells by cotransfection of four plasmids. pRCVP2koA encodes nonstructural proteins required for replication (rep) and the viral capsid proteins VP1 and VP3. Expression of vp2 was prevented by a silent mutation within the vp2 start codon T138 (Start^{mut}) and the natural HSPG tropism was ablated by two mutations (R585A, R588A, HSPG^{ko}). pMH303 encodes PIF6 fused to VP2 (HSPG^{ko}) under the CMV promoter. pCMVgfp contains a self-complementary AAV genome encoding GFP. pHelper provides adenoviral genes for AAV production. (C), Western Blot (B1 antibody) against capsid proteins of wild type (wt) AAV-2_{GFP} and OptoAAV_{GFP}. Representative data of n=3 batches. (D), Binding of OptoAAV_{GFP} to PhyB. OptoAAVs_{GFP} (optionally pre-incubated at 62.5° C. for 10 min) were incubated with or without PhyB-functionalized beads under 660 nm light for 1 h. After pelleting beads, supernatants were analyzed for unbound OptoAAV_{GFP} by Western Blot (WB, B1 antibody) against VP3. Representative data of n=4 experiments. ITR, inverted terminal repeat. M, protein size marker.

FIG. 3. Characterization of the OptoAAV system. (A), Heating of OptoAAV. A-431 cells were incubated with heat-treated OptoAAVS_{GFP} (MOI, genomic particles per cell: 3.4×10⁴) and 50 nM PhyB-DARPin_{EGFR} in PBS supplemented with 1% BSA for 2 h under 740 nm or pulsed 660 nm illumination. Afterwards, cells were washed and incubated in medium under continued illumination for 46 h before analysis of transduced (GFP-positive) cells by flow cytometry. n>5,600 cells/sample. (B), Influence of OptoAAV components. A-431 cells were incubated for 2 h in PBS supplemented with 1% BSA and 50 nM PhyB or PhyB-DARPin_{EGFR}, OptoAAV_{GFP} (MOI(+): 4.5×10⁴, MOI(++): 1.4×10⁵) or wild type (wt) AAV-2G FP (MOI: 9.3×10³). The experiment was performed as in (A) with OptoAAV_{GFP} pre-incubated at 62.5° C. for 10 min. n>300 cells/sample. (C), Dose dependency. A-431 cells incubated with 50 nM PhyB-DARPin_{EGFR} and OptoAAV_{GFP} (MOI: 2.8×10⁴) in PBS supplemented with 10% FCS were illuminated as indicated with 630 nm light and incubated afterwards in the dark. 2 h after illumination start, cells were washed and incubated in medium for 46 h under 740 nm light before flow cytometry analysis. Samples illuminated with 780 nm light within the first 2 h showed 0.21% GFP-positive cells. Values obtained from biological duplicates. n>3,800 cells/sample. Mean is indicated. n.s. P≥0.05, ***P<0.001, ****P<0.0001.

FIG. 4. Modular adaptation of the OptoAAV system to different cell types. (A), Transduction of different cell lines with PhyB-DARPin adapter proteins specific for the cell surface receptors EGFR, EpCAM, and Her2. The cell lines were incubated as indicated with 50 nM PhyB-DARPin and OptoAAV_{GFP} (MOI: 4.1×10⁴) in PBS supplemented with 10% FCS for 2 h under 740 nm or pulsed 660 nm illumination. Afterwards, cells were washed and incubated in medium under continued illumination for 46 h before analysis of transduced (GFP-positive) cells by flow cytometry. n>1,200 cells/sample. (B), Light-controlled transduction of primary human CD4+ T cells. T cells were incubated with 50 nM PhyB-DARPin_{CD4} and OptoAAV_{GFP} (MOI: 4.7×10⁴) in PBS supplemented with 10% FCS for 6 h under 740 nm or pulsed 660 nm illumination. Afterwards, cells were incubated in medium under continued illumination for 42 h. Following staining of the cells with a V450-labeled anti-CD4 antibody, the number of transduced (GFP-positive) CD4-positive and -negative cells was analyzed by flow cytometry. n>900 cells/sample. Mean is indicated. n.s. P≥0.05, ***P<0.001, ****P<0.0001.

FIG. 5. Spatiotemporally controlled transduction. (A), Transduction with one transgene. A-431 cells were incubated with 50 nM PhyB-DARPin_{EGFR} in PBS supplemented with 10% FCS under 740 nm light for 10 min. After washing, OptoAAVS_{GFP} (MOI: 1.7×10⁴) in PBS supplemented with 10% FCS were added and cells were illuminated for 9 s from the bottom with 660 nm light (15 μmol m⁻² s⁻¹) using the photomask with simultaneous global 740 nm illumination (3 μmol m⁻² s⁻¹) from the top. Following 2 h incubation in the dark, cells were washed and incubated for 46 h in medium under 740 nm light. Finally, cells were fixed, DAPI-stained and imaged by confocal microscopy. Representative images from n=4 experiments. (B), Transduction with two transgenes. A-431 cells were transduced spatially resolved as in (A) using photomask 1 and OptoAAV_{GFP} (MOI: 1.5×10⁴). Following incubation in the dark, the procedure was repeated using photomask 2 and OptoAAVm_{Scarlet} (MOI: 1.7×10⁴). After the second incubation step in the dark, cells were washed and incubated for 44 h in medium under 740 nm light. Finally, cells were fixed, DAPI-stained and imaged. The DAPI image is shown in FIG. S12C. Representative images from n=2 experiments. Scale bars, 1 mm.

FIG. 6. Spatially resolved transduction of single cells using a conventional confocal microscope. (A), Experimental workflow. A-431 cells containing 1% fluorescently labeled (CFSE or eFluor670) cells were seeded on a gridded coverslip. 48 h post seeding, cells were incubated with 50 nM PhyB-DARPin_{EGFR} in PBS supplemented with 10% FCS under 740 nm light for 10 min. After washing, OptoAAVS_{GFP} or OptoAAVs_{mScarlet} in PBS supplemented with 10% FCS were added and a bright field and CFSE or eFluor670 image was acquired under 740 nm illumination (200 μmol m⁻² s⁻¹). After switching the 740 nm light off, a single CFSE or eFluor670-positive cell was illuminated with the 633 nm laser of the confocal microscope. Following 2 h incubation in the dark, cells were washed and incubated for 46 h in medium under 740 nm light. Finally, cells were fixed, DAPI-stained and imaged by confocal microscopy. (B), Light-controlled transduction of a single CF SE-stained A-431 cell. The experiment was performed as in (A) with OptoAAVm_{Scarlet} (MOI: 4.9×10⁴). The area illuminated with the 633 nm laser is encircled in red. Representative images from n=9 successful experiments (out of 15). All experiments are shown in FIG. S4A. Scale bar, 100 μm.

FIG. S1. Analysis of different PhyB-DARPin adapter proteins by SDS-PAGE after immobilized metal affinity chromatography (IMAC) purification. The polyacrylamide gel was stained for the PhyB chromophore PCB (Zn²⁺-staining) and for proteins (Coomassie-staining). PhyB-DARPin_{EGFR} was loaded from two different expressions/purifications to assess batch-to-batch variability. Representative images from n=2 experiments. Calculated molecular weights: PhyB-DARPin_{EGFR}, 89.4 kDa; PhyB-DARPin_{EpCAM}, 89.8 kDa; PhyB-DARPin_{HER2}, 89.7 kDa; PhyB-DARPin_{CD4}, 87.3 kDa. M, protein size marker.

FIG. S2. Spectral analysis of the different purified PhyB-DARPin adapter proteins. Absorbance spectra of the depicted PhyB-DARPin proteins (0.5 mg ml⁻¹) in PBS after illumination with 660- or 740-nm light (both 100 μmol m⁻² s⁻¹) for 3 min (left panel). To obtain the corresponding difference spectra (right panel), the absorbance spectrum upon 660-nm illumination was subtracted from the spectrum after 740-nm illumination. The wavelengths of the isosbestic point as well as of the minimum/maximum in the difference spectrum are indicated. The different phytochrome (672 nm) to protein (280 nm) absorbance ratios were likely caused by the different purity of the adapter proteins (see FIG. S1). Representative data of n=2 experiments.

FIG. S3. Light-dependent protein interaction between PhyB-DARPin_{EGFR} and PIF6. PhyB-DARPin_{EGFR} (0.6 mg ml⁻¹) was mixed with an equimolar amount of mVenus-PIF6 and illuminated with 660- or 740-nm light (100 μmol m⁻² s⁻¹) for 3 min before analysis by size exclusion chromatography in the dark. The absorbance was monitored at 280 nm (protein), 514 nm (absorbance maximum of mVenus-PIF6) and at 672 nm (isosbestic point of PhyB-DARPin_{EGFR}). Calibration of the column with a protein size standard is indicated by the dotted line. mVenus-PIF6 is partially forming homodimers via cysteines due to the absence of reducing agent in the running buffer. Based on the standard, the apparent molecular weights of the indicated peaks were: PhyB-DARPin_{EGFR}+mVenus-PIF6, 191 kDa; PhyB-DARPin_{EGFR}, 108 kDa; mVenus-PIF6, 96.8 and 59.5 kDa. Calculated molecular weights based on the amino acid sequence: PhyB-DARPin_{EGFR}, 89.4 kDa; mVenus-PIF6, 39.6 kDa. Representative data of n=2 experiments.

FIG. S4. Binding of PhyB and PhyB-DARPin_{EGFR} to A-431 cells. Detached A-431 cells were incubated with 1 μM PhyB or PhyB-DARPin_{EGFR} in DMEM supplemented with 10% (v/v) FCS for 2 hours at 37° C. in the dark. After washing, PhyB fluorescence of the cells was analyzed by flow cytometry. n>6400 cells per sample.

FIG. S5. Light-controlled recruitment of mVenus-PIF6 to the cell surface. Adherent A-431 cells were incubated as indicated with 100 nM PhyB-DARPin_{EGFR} and 200 nM mVenus-PIF6 in PBS supplemented with 10% (v/v) FCS at 37° C. for 20 min or on ice for 1 hour under 740- or pulsed 660-nm illumination. After washing, cells were fixed with PFA, nuclei were stained with TO-PRO-3 and mVenus fluorescence of cells was analyzed by confocal microscopy. Representative images of n=3 experiments. Scale bar, 10 μm.

FIG. S6. Gating strategy of flow cytometry experiments analyzed in this study. The gating strategy of representative samples of the experiment depicted in FIG. 3A is shown. First, cells were gated based on FSC-A and SSC-A signals and doublets were excluded based on the FSC-A and FSC-H signal. Next, autofluorescent cells were excluded based on their fluorescent signal in an unused channel and finally GFP-negative and -positive cells were gated based on the blank control.

FIG. S7. Impact of heating of AAVs on transduction. (A) Impact of heating of OptoAAV_{GFP} on light-controlled transduction. OptoAAVS_{GFP} were exposed for the indicated times to 60° C. or 65° C. Next, light-controlled transduction of A-431 cells was assessed as described in FIG. 3A. n>3300 cells per sample. Mean is indicated. n.s., P≥0.05; **P<0.01; ***P<0.001; ****P<0.0001. (B) Impact of heating of wild type (WT) AAV-2_{GFP} on transduction. A-431 cells were incubated with untreated or heated (62.5° C. for 10 min) WT AAV-2_{GFP} at the indicated MOI in PBS supplemented with 10% (v/v) FCS for 2 hours. Afterward, cells were washed and incubated in medium for 46 hours before analysis of the ratio of transduced (GFP-positive) cells by flow cytometry. The right panel shows a zoom in for low MOIs of the left panel. There, a linear correlation between transduction and MOI was observed. To obtain a transduction of 10% GFP-positive cells, an MOI of 70 and 410 is required for untreated and heated WT AAV-2_{GFP}, respectively. This corresponds to a 5.9-fold loss of infectivity caused by the heat treatment. Data was fitted by linear regression. n>1200 cells per sample.

FIG. S8. Characterization of the OptoAAV system. (A) Impact of PhyB-DARPin_{EGFR} concentration on light-controlled transduction. A-431 cells were incubated for 2 hours with the indicated concentrations of PhyB-DARPin_{EGFR} and OptoAAV_{GFP} (MOI: 4.5×10⁴) in PBS supplemented with 1% (w/v) BSA under 740- or pulsed 660-nm illumination. Afterward, cells were washed and incubated in medium under continued illumination for 46 hours before analysis of the ratio of transduced (GFP-positive) cells by flow cytometry. n>900 cells per sample. (B) Characterization of the OptoAAV system in different buffers and media. A-431 cells were incubated for 2 hours with 50 nM PhyB-DARPin_{EGFR} and where indicated with OptoAAV_{GFP} (MOI: 4.5×10⁴) in the indicated buffers and media supplemented with different amounts of BSA (w/v) or FCS (v/v) under 740- or pulsed 660-nm illumination. Afterward, cells were washed and incubated in medium under continued illumination for 46 hours before analysis of the ratio of transduced (GFP-positive) cells by flow cytometry. n>2600 cells per sample. (C) Impact of OptoAAV_{GFP}/PhyB-DARPin_{EGFR} incubation time on light-controlled viral transduction. A-431 cells were incubated for different time periods with OptoAAV_{GFP} (MOI: 4.5×10⁴) and 50 nM PhyB-DARPin_{EGFR} in the indicated buffers and media under 740- or pulsed 660-nm illumination. Afterward, cells were washed and incubated in medium under continued illumination up to a total incubation time of 48 hours before analysis of the ratio of transduced (GFP-positive) cells by flow cytometry. n>2200 cells per sample. Mean is indicated. n.s., P≥0.05; *P<0.05; ***P<0.001; ****P<0.0001.

FIG. S9. Impact of the OptoAAV_{EGFR} system on EGFR activation. Serum-starved A-431 cells were incubated for 15 or 30 min as indicated with EGF (10 ng μP i), wild type (WT) AAV-2_{GFP} (MOI: 3.2×10⁴), PhyB-DARPin_{EGFR} (50 nM), or OptoAAV_{GFP} (MOI: 2.2×10⁴) in PBS supplemented with 1% (w/v) BSA under 740- or pulsed 660-nm illumination. Afterward, cells were lysed and phosphorylation of EGFR and Erk1/2 was analyzed by Western blotting using the indicated antibodies.

FIG. S10. Light-controlled transduction of different cell lines with OptoAAV_{GFP} and the adapter protein PhyB-DARPin_{EGFR}. Different cell lines were incubated as depicted with 50 nM PhyB-DARPin_{EGFR} and OptoAAV_{GFP} (A549 MOI: 6.8×10⁴; HeLa MOI: 3.4×10⁴; MDA-MB-231 MOI: 6.8×10⁴; CHO-K1 MOI: 6.8×10⁴) in different buffers (A549 and MDA-MB-231: PBS supplemented with 10% (v/v) FCS; HeLa and CHO-K1: PBS supplemented with 1% (w/v) BSA) for 2 hours under 740- or pulsed 660-nm illumination. Afterward, cells were washed and incubated in the respective medium of each cell line for 46 hours under continued illumination before analysis of the ratio of transduced (GFP-positive) cells by flow cytometry. Mean is indicated. n.s., P≥0.05; **P<0.01; ***P<0.001; ****P<0.0001.

FIG. S11. Analysis of the role of cellular target receptor internalization for the OptoAAV system. (A) HEK-293T cells stably expressing SpyCatcher fused to the transmembrane domain of MHC I (left panel) or A-431 cells (right panel) were incubated with 100 nM of PhyB-mCherry-SpyTag or FRB-mCherry-DARPin_{EGFR}, respectively, in PBS supplemented with 10% (v/v) FCS for 20 min and 2 hours at 37° C. After washing, cells were fixed with PFA, nuclei were stained with TO-PRO-3, and mCherry fluorescence was analyzed by confocal microscopy. Representative images of n=3 experiments. Scale bar, 10 μm. (B) Light-controlled transduction of HEK-293T cells displaying PhyB on the surface. If indicated, HEK-293T cells stably expressing SpyCatcher fused to the transmembrane domain of MHC I were incubated with 100 nM PhyB-mCherry-SpyTag for 30 min at 37° C. After washing, cells were incubated as indicated for 2 hours in medium supplemented with OptoAAV_{GFP} (MOI(+): 3.3×10²; MOI(++): 1.3×10³) or wild type (WT) AAV-2G FP (MOI: 2.5×10³) under 740- or pulsed 660-nm illumination. Afterward, cells were washed and incubated in medium under continued illumination for 46 hours before analysis of the transduced (GFP-positive) cells by flow cytometry. n>1600 cells per sample. Mean is indicated. n.s., P≥0.05; ****P<0.0001.

FIG. S12. Spatiotemporally resolved transduction with one (A) or two (B) different transgenes. (A) A-431 cells were incubated with 50 nM PhyB-DARPin_{EGFR} in PBS supplemented with 10% (v/v) FCS under 740-nm illumination for 10 min. After washing, OptoAAVs_{GFP} (MOI: 1.9×10⁴) or OptoAAVs_{mScarlet} (MOI: 9.1×10³) in PBS supplemented with 10% (v/v) FCS were added and cells were illuminated for 9 s from the bottom with 660-nm light (15 μmol m⁻² s⁻¹) using the depicted photomask with simultaneous global 740-nm illumination (3 μmol m's⁻¹) from the top. Following 2 hours of incubation in the dark, cells were washed and incubated for 46 hours in medium under 740-nm illumination before fixation of the cells, DAPI staining and visualization of GFP, mScarlet and DAPI fluorescence by confocal microscopy. Representative images from n=3 experiments. (B) A-431 cells were transduced spatially resolved as described in (A) using the depicted photomask 1 and OptoAAVm_{Scarlet} (MOI: 1.8×10⁴). Following incubation in the dark and a washing step, the same procedure was repeated using the depicted photomask 2 and OptoAAV_{GFP} (MOI: 1.7×10⁴). After the second incubation step in the dark, cells were washed and incubated for 44 hours in medium under 740-nm illumination. Next, cells were fixed, DAPI-stained and GFP, mScarlet and DAPI fluorescence was visualized by confocal microscopy. The lower panel shows a magnified selection of the upper mScarlet/GFP fluorescence image. Representative images from n=2 experiments. Scale bar of the lower panel, 20 μm. (C) DAPI image of experiment described in FIG. 5B. Scale bars, 1 mm (except lower panel in (B)).

FIG. S13. Light-controlled transduction of eFluor670-stained cells. (A) Light-controlled transduction of a selected single eFluor670-stained A-431 cell. The experiment was performed as described in FIG. 6A with OptoAAV_{GFP} (MOI: 5.0×10⁴). The area illuminated with the 633-nm laser is encircled in red. Representative images from n=4 successful experiments (out of 17). Scale bar, 100 μm (B) Light-controlled transduction of unlabeled and eFluor670-stained cells. A mixed culture of unlabeled (50%) and eFluor670-stained (50%) A-431 cells was incubated for 2 hours with 50 nM PhyB-DARPin_{EGFR} and OptoAAVm_{Scarlet} (MOI: 4.9×10⁴) in PBS supplemented with 10% (v/v) FCS under 740- or pulsed 660-nm illumination. Afterward, cells were washed and incubated in medium under continued illumination for 46 hours before analysis of the ratio of transduced (mScarlet-positive) cells by flow cytometry. n>850 cells per sample. Mean is indicated. n.s., P≥0.05; ****P<0.0001.

FIG. S14. Light-controlled transduction of CFSE-stained cells. (A) Light-controlled transduction of selected single CF SE-stained A-431 cells. The experiments were performed as described in FIG. 6B with OptoAAVm_{Scarlet}. The cells illuminated with the 633-nm laser are highlighted by arrows. The counted nonilluminated mScarlet-positive cells are partially only visible under adjusted contrast. Scale bar, 100 μm (B) Quantification of mScarlet-positive nonilluminated cells from the successful single-cell experiments in (A). For comparison, the percentages of mScarlet-positive and CFSE-negative cells under 740-nm illumination in the comparable flow cytometry experiment depicted in (C) are plotted. (C) Light-controlled transduction of unlabeled and CF SE-stained cells. A mixed culture of unlabeled (50%) and CFSE-stained (50%) A-431 cells was incubated with 50 nM PhyB-DARPin_{EGFR} and OptoAAVm_{Scarlet} (MOI: 4.9×10⁴) in PBS supplemented with 10% (v/v) FCS. Alternatively, the mixed cell culture was first incubated for 10 min with 50 nM PhyB-DARPin_{EGFR} under 740-nm illumination and after washing with OptoAAV_{mScarlet} (MOI: 4.9×10⁴) similar to the microscopy experiments in (A). After addition of the OptoAAV, the samples were incubated under 740- or pulsed 660-nm light for 2 hours. Afterward, cells were washed and incubated in medium under 740-nm illumination for 46 hours before analysis of the ratio of transduced (mScarlet-positive) cells by flow cytometry. n>800 cells per sample. Mean is indicated. n.s., P≥0.05; ***P<0.001; ****P<0.0001.

FIG. T1. Overview of light-controlled viral transduction systems.

FIG. T2. Nucleic acid sequences of plasmids generated in the example.

1. A kit of parts having biological activity, the kit of parts comprising
a first target comprising a first protein,
a second target comprising a second protein,
wherein the first protein and the second protein are suitable to form a heterodimer upon irradiation with UV, visible or infrared light in a first wavelength range or in the dark, which can optionally be reversed upon irradiating the heterodimer with UV, visible or infrared light in a second wavelength range or in the dark, wherein the second wavelength range is different from the first wavelength range, wherein the biological activity consists of triggering both the uptake of DNA, RNA, proteins, or small molecules into a cell which is preferably genetically unmodified, and biological effects, and
characterized in that at least one of the first and the second target itself has reduced biological activity as compared with the heterodimer.

2. The kit of parts of claim 1, wherein the first target is suitable to bind to a lipid or protein or combinations thereof, preferably a receptor or part thereof, that is expressed on a cell, preferably a genetically unmodified cell.

3. The kit of parts of claim 2, wherein the first target's binding to the lipid or protein or combinations thereof, preferably the receptor or part thereof, does not trigger biological effects.

4. The kit of parts of any one of claims 1 to 3, wherein the second target is essentially not suitable to adsorb or bind to a cell, preferably a genetically unmodified cell.

5. The kit of parts of any one of claims 1 to 4, wherein the second target comprises at least one molecule that is suitable to lead to a change in the molecular process of gene expression, DNA synthesis, gene silencing, RNA synthesis, preferably gene transcription, protein synthesis, peptide synthesis, post-translational modifications, preferably glycosylation, phosphorylation, ubiquitinylation, sumoylation, methylation or acetylation; cell growth; autophagy; mitophagy; cell death; cell division; cell proliferation; cell survival; cell differentiation; cell ageing; organelle growth; organelle proliferation; polymerization or depolymerization of the cytoskeleton; viral replication; reverse transcription; phosphorylation of nucleosides; phosphorylation of nucleotides; apoptosis, necrosis, or antigen presentation.

6. The kit of parts of any one of claims 1 to 5, wherein the second target comprises a liposome, an exosome, a DNA; preferably a viral DNA and/or a suicide gene; an RNA, preferably a viral RNA; a protein, preferably a reverse transcriptase, a viral thymidine kinase, a viral cytidine kinase, a viral adenine kinase, a viral guanine kinase, a virulence factor, a viral integrase, a protease, an apoptotic factor, a hormone having a nuclear receptor, preferably a steroid hormone, thyroid hormone, vitamin D, retinoic acid, NGF1-b; SF1-like protein, or GCNF-like protein; a transcription factor; a small molecule, preferably an active ingredient.

7. The kit of parts of any one of claims 1 to 6, wherein the second target comprises a viral protein or derivatives thereof, preferably retroviral, lentiviral, adenoviral, adeno-associated viral, vesicular stomatitis viral, Newcastle Disease viral, herpes simplex viral, measles viral, pox viral, alphaviral, flaviral, rhabdoviral, picornaviruses or baculoviral protein or derivatives thereof, more preferably an AAV-2 protein or derivatives thereof.

8. The kit of parts of any one of claims 1 to 7, wherein the second target comprises a fusion protein comprising the second protein, wherein the fusion protein preferably comprises the full sequence or a partial sequence of a viral capsid protein, including VP1, VP2 and VP3 proteins of AAV.

9. The kit of parts of claim 8, wherein the VP1, VP2 and VP3 proteins bear the mutations R585A and R588A.

10. The kit of parts of any one of claims 1 to 9, wherein the first protein is a phytochrome and the second protein is a phytochrome interacting partner or vice versa.

11. The kit of parts of claim 10, wherein the phytochrome is selected from the group comprising PhyA, PhyB, PhyC, PhyD, PhyE, BphP1 and DrBphP and the phytochrome interacting partner is selected from the group comprising PIF1, PIF2, PIF3, PIF4, PIF5, PIF6, PIF7, PIF8, FHY1, FHL, PpsR2, Q-PAS1 and engineered antibodies (or fragments thereof).

12. Use of the kit of parts of any one of claims 1 to 11 for importing the second target into a membrane-bound compartment, preferably a cell, more preferably an animal cell.

13. Use according to claim 12, wherein the second target comprises a virus or a derivative thereof, preferably AAV-2 or a derivative thereof.

14. A method for importing DNA, RNA, protein or small molecule into a cell which is preferably genetically unmodified, comprising
(i) preparing a kit of parts of any one of claims 1 to 11;
(ii) bringing the kit of parts into contact with the cell,
irradiating the kit of parts with visible or infrared light in a first wavelength range to obtain a heterodimer of the first protein and the second protein.

15. A kit of parts comprising
a first nucleotide sequence encoding a first target protein,
a second nucleotide sequence encoding a second target protein,
wherein the first target protein and the second target protein are suitable for assembly into a first target and a second target as defined in claim 1, respectively,
wherein the first target is coupled to a first protein through the first target protein and the second target is coupled to a second protein through the second target protein, and
wherein the first protein and the second protein are suitable to form a heterodimer upon irradiation with visible or infrared light in a first wavelength range, which can optionally be reversed upon irradiating the heterodimer with visible or infrared light in a second wavelength range, wherein the second wavelength range is different from the first wavelength range.
